(19) United States
(12) Patent Application Publication
(10) Pub. No.: US 2026/0139057 A1
(43) Pub. Date: May 21, 2026

(54) HUMANIZED ANTIBODIES TO HUMAN INTEGRIN AVB8

(71) Applicant: The Regents of the University of California, Oakland, CA (US)

(72) Inventors: Stephen Nishimura, Mill Valley, CA (US); Mingliang Jin, Oakland, CA (US); Robert Seed, Redwood City, CA (US); Jianlong Lou, San Bruno, CA (US); James Marks, Oakland, CA (US); Alexis Combes, San Francisco, CA (US); Jody L. Baron, Mill Valley, CA (US); Yifan Cheng, Oakland, CA (US)

(73) Assignee: The Regents of the University of California, Oakland, CA (US)

(21) Appl. No.: 18/872,234

(22) PCT Filed: Jun. 12, 2023
(86) PCT No.: PCT/US2023/025080

§ 371 Date: Dec. 5, 2024

Related U.S. Application Data
(60) Provisional application No. 63/351,731, filed on Jun. 13, 2022.

(51) Int. Cl. C07K 16/28; A61K 39/00; A61P 35/00; C07K 14/725

(52) U.S. Cl. CPC C07K 16/2839; A61P 35/00; C07K 14/7051; A61K 2039/505; C07K 2317/24; C07K 2317/565; C07K 2317/622; C07K 2317/71; C07K 2317/73; C07K 2317/76; C07K 2317/92

(57) ABSTRACT

Antibodies with improved affinity for integrin avB8 and uses thereof are provided.

### CROSS-REFERENCE TO RELATED PATENT APPLICATIONS

The present application claims benefit of priority to U.S. Provisional Patent Application No. 63/351,731, filed Jun. 13, 2022, which is incorporated by reference for all purposes.

### STATEMENT AS TO RIGHTS TO INVENTIONS MADE UNDER FEDERALLY SPONSORED RESEARCH AND DEVELOPMENT

This invention was made with government support under grant no. R01HL134183 and P41CA196276, both awarded by The National Institutes of Health. The government has certain rights in the invention.

### BACKGROUND OF THE INVENTION

Transforming growth factor β (TGFβ) was originally characterized as an oncogene capable of inducing a transformed phenotype in non-neoplastic cells. A number of TGFβ family members have since been characterized, based on the presence of similar amino acid domains.

Three TGF-β isoforms are expressed ubiquitously in mammals (TGF-β 1-3), but are maintained in an inactive form by non-covalent interaction with a propeptide, the latency associated domain of TGF-β (LAP). For TGF-β to signal, it must be released from its inactive complex by a process called TGF-β activation. The small latent TGF complex (SLC) includes 2 components: the active (mature) TGF-β homodimer and latency associated peptide (LAP). LAP is a homodimer, linked by disulfide bonds, that represents the N-terminal end of the TGF-β precursor protein. The mature TGF-β protein represents the C terminal end (about 25 kD) of the precursor. The bond between the TGF-βs and LAP is proteolytically cleaved within the Golgi, but the TGF-β propeptide remains bound to TGF-β by non-covalent interactions to form the SLC. LAP envelops that mature TGF-β homodimer within a ring-like structure, which prevents mature TGF-β from interacting with its receptors thus conferring latency. LAP-TGFβ binding is reversible and the isolated purified components can recombine to form an inactive SLC. The SLC is herein referred to as latent TGF-β, (L-TGF-β).

The SLC is poorly secreted and for efficient secretion requires association with a binding partner, which may take the form of an extracellular matrix molecule, latent transforming growth factor-β binding protein (LTBP) or a type I transmembrane protein such as Leucine Rich Repeat Containing 32 (LRRC32) also called Glycoprotein A Repetitions Predominant (GARP), the latter which is enriched on the surface of suppressor CD4 T-cells (Treg). GARP plays a role in presentation of L-TGF-β to several integrins, which is essential for L-TGF-β function in vivo (Seed et al. (2021) Sci Immunol. 6:57).

In general, integrins are adhesion molecules and mediate the attachment of cells to extracellular matrix proteins. Integrin αvβ8 binds to the LAP of TGF-β and mediates the activation of TGF-β1 and 3 (Mu et al. (2002) J. Cell Biol. 159:493). Integrin αvβ8-mediated activation of TGF-β is required for in vivo activation of TGF-β (i.e., release of the mature TGF-β polypeptide), thus αvβ8 is a gatekeeper of TGF-β function. Integrin αvβ8 is expressed in normal epithelia (e.g., airway epithelia), mesenchymal cells, and neuronal tissues.

The integrin β8 (Itgb8) has been shown to play a role in expression of forkhead box P3 (Foxp3), which supports Treg differentiation and specific epigenetic remodeling. See, e.g., Vandenbon, et al., Proc. Natl. Acad. Sci. USA vol. 113 no. 17 pp. E2393-E2402 (2016). Some studies suggest human and mouse effector Treg cells express functional TGF-β-activating integrin αvβ8. See, Worthington, Immunity Volume 42, Issue 5, pp. 903-915 (May 2015). Other studies demonstrate that tumor cells are the major site of αvβ8 expression in the tumor microenvironment, where tumor cell αvβ8 lead to accumulation of immunosuppressive T-cells (regulatory T-cells (Treg), and M2 macrophages) at the same time decreasing the numbers of intratumoral cytotoxic CD8+ T-cells (Takasaka, et al. JCI Insight (2018) 3; 20). Tumor cells expressing αvβ8 lead to intratumoral conversion of TGF-β/GARP presenting T-cells to Treg, which enhances tumor immune evasion through suppression of cytotoxic T-cell responses (Seed et al. (2021) Sci Immunol. 6:57). Since TGF-β is a major mechanism of resistance to checkpoint inhibitors, and inhibition αvβ8 enhances response to checkpoint inhibitors, targeting αvβ8-mediated TGF-β activation is a method available for treating a variety of human cancers (Takasaka, et al. JCI Insight (2018) 3; 20).

TGF-β is also one of the most fibrogenic cytokines expressed in mammals. Thus, integrin αvβ8 expression by epithelial cells and mesenchymal cells indicates that targeting αvβ8-mediated TGF-β activation is also a possible therapeutic approach for inhibiting fibrosis in organs where αvβ8 is expressed, such as lung (Kitamura, et al. JCI (2011) 121, 7:2863-75), liver and kidney (Takasaka, et al. JCI Insight (2018) 3; 20).

Antibodies that bind to αvβ8 have been described, for example, in PCT Publications WO 2021/146614 and WO2018/064478.

### BRIEF SUMMARY OF THE INVENTION

In some embodiments, an antibody that specifically binds human αvβ8 is provided. In some embodiments, the antibody comprises:
(a) a heavy chain variable region comprising a heavy chain complementary determining region (HCDR) 1 comprising RQSMH (SEQ ID NO:1) or EQSMH (SEQ ID NO:7);
a HCDR 2 comprising RINTETGEPTYAQKFQG (SEQ ID NO:2);
a HCDR 3 comprising FYYGRD (S/T) (SEQ ID NO:3); and
(b) a light chain variable region comprising a light chain complementary determining region (LCDR) 1 comprising RSSQSLLHSRSRKNYLA (SEQ ID NO:4)
a LCDR2 comprising WASTRET (SEQ ID NO:5); and
a LCDR3 comprising KQSYNLLS (SEQ ID NO:6).

In some embodiments, the heavy chain variable region comprises: SEQ ID NO:34; and
the light chain variable region comprises: SEQ ID NO:35; or
the heavy chain variable region comprises: SEQ ID NO:34; and
the light chain variable region comprises: SEQ ID NO:36; or
the heavy chain variable region comprises: SEQ ID NO:37; and
the light chain variable region comprises: SEQ ID NO:35; or
the heavy chain variable region comprises: SEQ ID NO:37; and
the light chain variable region comprises: SEQ ID NO:36; or
the heavy chain variable region comprises: SEQ ID NO:38; and
the light chain variable region comprises: SEQ ID NO:35; or
the heavy chain variable region comprises: SEQ ID NO:38; and
the light chain variable region comprises: SEQ ID NO:36; or
the heavy chain variable region comprises: SEQ ID NO:39; and
the light chain variable region comprises: SEQ ID NO:35; or
the heavy chain variable region comprises: SEQ ID NO:39; and
the light chain variable region comprises: SEQ ID NO:36.

In some embodiments, the antibody is humanized. In some embodiments, the isotype of the antibody is IgG1, IgG2, IgG3 or IgG4. In some embodiments, the antibody comprises a constant (Fc) domain and the Fc comprises at least one mutation that reduces effector function, and or decreases aggregation or increases stability.

In some embodiments, the antibody is linked to a detectable label.

In some embodiments, the antibody is a tetrameric antibody, a Fab, or scfv.

In some embodiments, the antibody comprises:
(a) a heavy chain variable region comprising a heavy chain complementary determining region (HCDR) 1 comprising SEQ ID NO:1 or SEQ ID NO:7;
a HCDR 2 comprising SEQ ID NO:2;
a HCDR 3 comprising SEQ ID NO:3; and
(b) a light chain variable region comprising a light chain complementary determining region (LCDR) 1 comprising KSSQSLLGRGDLGRLKKNALA (SEQ ID NO:8) or RSSQSLLRRGDLATIHGNALA (SEQ ID NO:9) or KSSQSLLRRGDLATIHGNALA (SEQ ID NO: 10) or RSSQSLLRRGDLATIHGNALA (SEQ ID NO:11);
a LCDR2 comprising SEQ ID NO:5; and
a LCDR3 comprising SEQ ID NO:6.

In some embodiments,
the heavy chain variable region comprises: SEQ ID NO:34; and
the light chain variable region comprises: SEQ ID NO:40; or
the heavy chain variable region comprises: SEQ ID NO:34; and
the light chain variable region comprises: SEQ ID NO:41; or
the heavy chain variable region comprises: SEQ ID NO:37; and
the light chain variable region comprises: SEQ ID NO:40; or
the heavy chain variable region comprises: SEQ ID NO:37; and
the light chain variable region comprises: SEQ ID NO:41; or
the heavy chain variable region comprises: SEQ ID NO:34; and
the light chain variable region comprises: SEQ ID NO:42; or
the heavy chain variable region comprises: SEQ ID NO:34; and
the light chain variable region comprises: SEQ ID NO:43; or
the heavy chain variable region comprises: SEQ ID NO:37; and
the light chain variable region comprises: SEQ ID NO:42; or
the heavy chain variable region comprises: SEQ ID NO:37; and
the light chain variable region comprises: SEQ ID NO:43; or
the heavy chain variable region comprises: SEQ ID NO:38; and
the light chain variable region comprises: SEQ ID NO:40; or
the heavy chain variable region comprises: SEQ ID NO:38; and
the light chain variable region comprises: SEQ ID NO:41; or
the heavy chain variable region comprises: SEQ ID NO:39; and
the light chain variable region comprises: SEQ ID NO:40; or
the heavy chain variable region comprises: SEQ ID NO:39; and
the light chain variable region comprises: SEQ ID NO:41; or
the heavy chain variable region comprises: SEQ ID NO:38; and
the light chain variable region comprises: SEQ ID NO:42; or
the heavy chain variable region comprises: SEQ ID NO:38; and
the light chain variable region comprises: SEQ ID NO:43; or
the heavy chain variable region comprises: SEQ ID NO:39; and
the light chain variable region comprises: SEQ ID NO:42; or
the heavy chain variable region comprises: SEQ ID NO:39; and
the light chain variable region comprises: SEQ ID NO:43; or
the heavy chain variable region comprises: SEQ ID NO:34; and
the light chain variable region comprises: SEQ ID NO:44; or
the heavy chain variable region comprises: SEQ ID NO:34; and
the light chain variable region comprises: SEQ ID NO:45; or
the heavy chain variable region comprises: SEQ ID NO:37; and
the light chain variable region comprises: SEQ ID NO:44; or
the heavy chain variable region comprises: SEQ ID NO:37; and
the light chain variable region comprises: SEQ ID NO:45; or
the heavy chain variable region comprises: SEQ ID NO:34; and
the light chain variable region comprises: SEQ ID NO:46; or
the heavy chain variable region comprises: SEQ ID NO:34; and
the light chain variable region comprises: SEQ ID NO:47; or
the heavy chain variable region comprises: SEQ ID NO:37; and
the light chain variable region comprises: SEQ ID NO:46; or
the heavy chain variable region comprises: SEQ ID NO:37; and
the light chain variable region comprises: SEQ ID NO:47.
the heavy chain variable region comprises: SEQ ID NO:38; and
the light chain variable region comprises: SEQ ID NO:44; or
the heavy chain variable region comprises: SEQ ID NO:38; and
the light chain variable region comprises: SEQ ID NO:45; or
the heavy chain variable region comprises: SEQ ID NO:39; and
the light chain variable region comprises: SEQ ID NO:44; or
the heavy chain variable region comprises: SEQ ID NO:39; and
the light chain variable region comprises: SEQ ID NO:45; or
the heavy chain variable region comprises: SEQ ID NO:38; and
the light chain variable region comprises: SEQ ID NO:46; or
the heavy chain variable region comprises: SEQ ID NO:38; and
the light chain variable region comprises: SEQ ID NO:47; or
the heavy chain variable region comprises: SEQ ID NO:39; and
the light chain variable region comprises: SEQ ID NO:46; or
the heavy chain variable region comprises: SEQ ID NO:39; and
the light chain variable region comprises: SEQ ID NO:47.

In some embodiments, the antibody is humanized. In some embodiments, the isotype of the antibody is IgG1, IgG2, IgG3 or IgG4. In some embodiments, the antibody comprises a constant (Fc) domain and the Fc comprises at least one mutation that completely eliminates or reduces effector function and/or at least one mutation that reduces aggregation and/or improves stability.

In some embodiments, the antibody is linked to a detectable label.

In some embodiments, the antibody is a tetrameric antibody, a Fab, or scfv.

Also provided are methods of reducing TGFβ activation in a human. In some embodiments, the method comprises administering an antagonist of integrin αvβ8 to an individual in need thereof, wherein the antagonist is the antibody as described above or elsewhere herein, thereby reducing TGFβ activation in the individual.

In some embodiments, the antagonist inhibits exposure of active, mature TGFβ peptide, from the latent-TGF-β complex, but does not inhibit TGFβ once TGFβ is released from the latent-TGF-β complex.

In some embodiments, the human has at least one condition selected from the group consisting of chronic obstructive pulmonary disease (COPD), asthma, arthritis, a fibrotic disorder, an inflammatory brain autoimmune disease, multiple sclerosis, a demylinating disease (e.g., transverse myelitis, Devic's disease, Guillain-Barré syndrome), neuroinflammation, and kidney disease, and wherein TGF□ reduction results in amelioration of the condition. In some embodiments, the fibrotic disorder is selected from the group consisting of airway fibrosis, idiopathic pulmonary fibrosis, non-specific interstitial pneumonia, post-infectious lung fibrosis, diffuse alveolar damage, collagen-vascular disease associated lung fibrosis, drug-induced lung fibrosis, silicosis, asbestos-related lung fibrosis, respiratory bronchiolitis, respiratory bronchiolitis interstitial lung disease, desquamative interstitial fibrosis, cryptogenic organizing pneumonia, chronic hypersensitivity pneumonia, drug-related lung fibrosis, renal fibrosis, and liver fibrosis. In some embodiments, the method further comprises before the administering, measuring expression of itgb8 transcript or integrin β8 protein in a sample from the individual.

Also provided is a method of treating cancer in a human individual. In some embodiments, the method comprises administering a sufficient amount of the antibody as described above or elsewhere herein to the individual, thereby treating the cancer.

In some embodiments, the cancer is bladder cancer, colorectal cancer, glioblastoma, gynecologic cancer, liver cancer, head and neck cancer, kidney cancer, lung cancer, skin cancer, pancreas cancer, or sarcoma. In some embodiments, the cancer is lung squamous, lung adenocarcinoma, head and neck, melanoma, ovarian, endometrial, uterine stromal, endocervical, cervical, vulvar, fallopian, breast, endometrioid, skin squamous, prostatic, colon, gastric, upper aerodigestive cancers, sarcoma or glioma. In some embodiments, the cancer is a metastatic cancer. In some embodiments, the cancer is a primary cancer.

In some embodiments, tumors in the individual lack immune cell infiltration. In some embodiments, the cancer is selected from the group consisting of melanoma, gynecologic cancer, head and neck carcinoma, and sarcoma. In some embodiments, before the administering, the individual is treated with an initial treatment that reduces expression of β8 in cancer cells. In some embodiments, the initial treatment is selected from one or more of a checkpoint inhibitor (e.g., anti-PD-1, anti-PD-L1, anti-CTLA4, anti-LAG3, anti-TIM3, anti-TIGIT treatment), chemotherapy, cryo-therapy, radiation therapy, cell therapy (e.g., chimeric antigen receptor therapy), and immunomodulator treatment.

In some embodiments, the method further comprises before the administering, measuring expression of itgb8 transcript or integrin β8 protein in a sample from the individual.

Also provided is a method of enhancing an immune response to a viral infection in a human individual. In some embodiments, the method comprises administering a sufficient amount of the antibody as described above or elsewhere herein to the individual, thereby enhancing an immune response to the viral infection. In some embodiments, the viral infection is a hepatitis infection. In some embodiments, the viral infection is a hepatitis B infection.

Also provided is a pharmaceutical composition comprising the antibody as described above or elsewhere herein in a pharmaceutically acceptable excipient.

Also provided is a method of detecting the presence, absence, or quantity of human in a sample. In some embodiments, the method comprises contacting the antibody as described above or elsewhere herein to the sample, and detecting or quantifying binding of the antibody to the sample.

Also provided is a nucleic acid encoding a chimeric antigen receptor (CAR) polypeptide, wherein the CAR polypeptide comprises an extracellular domain comprising a single-chain antibody comprising:
(a) a heavy chain variable region comprising a heavy chain complementary determining region (HCDR) 1 comprising RQSMH (SEQ ID NO:1) or EQSMH (SEQ ID NO:7);
a HCDR 2 comprising RINTETGEPTYAQKFQG (SEQ ID NO:2);
a HCDR 3 comprising FYYGRD (S/T) (SEQ ID NO:3); and
(b) a light chain variable region comprising a light chain complementary determining region (LCDR) 1 comprising RSSQSLLHSRSRKNYLA (SEQ ID NO:4)
a LCDR2 comprising WASTRET (SEQ ID NO:5); and
a LCDR3 comprising KQSYNLLS (SEQ ID NO:6);
or
(c) a heavy chain variable region comprising a heavy chain complementary determining region (HCDR) 1 comprising SEQ ID NO: 1 or SEQ ID NO:7;
a HCDR 2 comprising SEQ ID NO:2;
a HCDR 3 comprising SEQ ID NO:3; and
(d) a light chain variable region comprising a light chain complementary determining region (LCDR) 1 comprising KSSQSLLGRGDLGRLKKNALA (SEQ ID NO:8) or RSSQSLLRRGDLATIHGNALA (SEQ ID NO:9) or KSSQSLLRRGDLATIHGNALA (SEQ ID NO: 10) or RSSQSLLRRGDLATIHGNALA (SEQ ID NO:11);
a LCDR2 comprising SEQ ID NO:5; and
a LCDR3 comprising SEQ ID NO:6.

In some embodiments, the heavy chain variable region comprises: SEQ ID NO:34; and
the light chain variable region comprises: SEQ ID NO:35; or
the heavy chain variable region comprises: SEQ ID NO:34; and
the light chain variable region comprises: SEQ ID NO:36; or
the heavy chain variable region comprises: SEQ ID NO:37; and
the light chain variable region comprises: SEQ ID NO:35; or
the heavy chain variable region comprises: SEQ ID NO:37; and
the light chain variable region comprises: SEQ ID NO:36; or
the heavy chain variable region comprises: SEQ ID NO:38; and
the light chain variable region comprises: SEQ ID NO:35; or
the heavy chain variable region comprises: SEQ ID NO:38; and
the light chain variable region comprises: SEQ ID NO:36; or
the heavy chain variable region comprises: SEQ ID NO:39; and
the light chain variable region comprises: SEQ ID NO:35; or
the heavy chain variable region comprises: SEQ ID NO:39; and
the light chain variable region comprises: SEQ ID NO:36.
In some embodiments, the heavy chain variable region comprises: SEQ ID NO:34; and
the light chain variable region comprises: SEQ ID NO:40; or
the heavy chain variable region comprises: SEQ ID NO:34; and
the light chain variable region comprises: SEQ ID NO:41; or
the heavy chain variable region comprises: SEQ ID NO:37; and
the light chain variable region comprises: SEQ ID NO:40; or
the heavy chain variable region comprises: SEQ ID NO:37; and
the light chain variable region comprises: SEQ ID NO:41; or
the heavy chain variable region comprises: SEQ ID NO:34; and
the light chain variable region comprises: SEQ ID NO:42; or
the heavy chain variable region comprises: SEQ ID NO:34; and
the light chain variable region comprises: SEQ ID NO:43; or
the heavy chain variable region comprises: SEQ ID NO:37; and
the light chain variable region comprises: SEQ ID NO:42; or
the heavy chain variable region comprises: SEQ ID NO:37; and
the light chain variable region comprises: SEQ ID NO:43; or
the heavy chain variable region comprises: SEQ ID NO:38; and
the light chain variable region comprises: SEQ ID NO:40; or
the heavy chain variable region comprises: SEQ ID NO:38; and
the light chain variable region comprises: SEQ ID NO:41; or
the heavy chain variable region comprises: SEQ ID NO:39; and
the light chain variable region comprises: SEQ ID NO:40; or
the heavy chain variable region comprises: SEQ ID NO:39; and
the light chain variable region comprises: SEQ ID NO:41; or
the heavy chain variable region comprises: SEQ ID NO:38; and
the light chain variable region comprises: SEQ ID NO:42; or
the heavy chain variable region comprises: SEQ ID NO:38; and
the light chain variable region comprises: SEQ ID NO:43; or
the heavy chain variable region comprises: SEQ ID NO:39; and
the light chain variable region comprises: SEQ ID NO:42; or
the heavy chain variable region comprises: SEQ ID NO:39; and
the light chain variable region comprises: SEQ ID NO:43; or
the heavy chain variable region comprises: SEQ ID NO:34; and
the light chain variable region comprises: SEQ ID NO:44; or
the heavy chain variable region comprises: SEQ ID NO:34; and
the light chain variable region comprises: SEQ ID NO:45; or
the heavy chain variable region comprises: SEQ ID NO:37; and
the light chain variable region comprises: SEQ ID NO:44; or
the heavy chain variable region comprises: SEQ ID NO:37; and
the light chain variable region comprises: SEQ ID NO:45; or
the heavy chain variable region comprises: SEQ ID NO:34; and
the light chain variable region comprises: SEQ ID NO:46; or
the heavy chain variable region comprises: SEQ ID NO:34; and
the light chain variable region comprises: SEQ ID NO:47; or
the heavy chain variable region comprises: SEQ ID NO:37; and
the light chain variable region comprises: SEQ ID NO:46; or
the heavy chain variable region comprises: SEQ ID NO:37; and
the light chain variable region comprises: SEQ ID NO:47.
the heavy chain variable region comprises: SEQ ID NO:38; and
the light chain variable region comprises: SEQ ID NO:44; or
the heavy chain variable region comprises: SEQ ID NO:38; and
the light chain variable region comprises: SEQ ID NO:45; or
the heavy chain variable region comprises: SEQ ID NO:39; and
the light chain variable region comprises: SEQ ID NO:44; or
the heavy chain variable region comprises: SEQ ID NO:39; and
the light chain variable region comprises: SEQ ID NO:45; or
the heavy chain variable region comprises: SEQ ID NO:38; and
the light chain variable region comprises: SEQ ID NO:46; or
the heavy chain variable region comprises: SEQ ID NO:38; and
the light chain variable region comprises: SEQ ID NO:47; or
the heavy chain variable region comprises: SEQ ID NO:39; and
the light chain variable region comprises: SEQ ID NO:46; or
the heavy chain variable region comprises: SEQ ID NO:39; and
the light chain variable region comprises: SEQ ID NO:47.

In some embodiments, the antibody is humanized.

Also provided is an immune cell comprising the nucleic acid and expressing the CAR polypeptide as described above or elsewhere herein. In some embodiments, the immune cell is a T-cell, a tumor infiltrating cell (TIL) or a natural killer cell.

### BRIEF DESCRIPTION OF THE DRAWINGS

FIG. 1: The C6D4 epitope has superior efficacy to other anti-αvβ8 epitopes. A) TGF-β activation is most effectively inhibited using an antibody to the C6D4 epitope. The αvβ8 ectodomain (1 ug/ml) was coated on 96 well plates and incubated at 37 deg for 1 hr before washing with PBS with Ca2+ and Mg2+ followed by a blocking step (BSA, 1 mg/ml). The TMLC cells either expressing or not expressing GARP (SEQ ID NO:54) and TGF-β (SEQ ID NO: 50) were added to wells in Dulbecco's Modified Eagles Media (DMEM) with 10% fetal calf serum (FCS) with no antibody or the indicated antibody inhibitors SV5 (control IgG2a), 37E1B5 (Minigawa, et al (2014) Sci Transl Med, 6 (241), C6D4 (Takaska, et al (2018) JCI Insight, 3 (20)), and ADWA11 2.4 (from Pat no. US2020/0079855 A1), at the indicated concentrations (ug/ml) and incubated overnight at 37 deg in a humidified CO2 incubator. The luciferase values from the TMLC control sample treated with each respective condition was subtracted from TMLC GARP/TGF-β samples treated with the same antibody. Activation (%) is reported as luciferase units with antibody treated sample/no antibody control×100. The EC50 of each antibody is shown below in μg/ml (n=3). B) C6D4 is most effective at inhibiting adhesion to αvβ8 coated wells. The same assay format as in A was used to measure the attachment of TMLC GARP/TGF-β expressing cells to wells coated with αvβ8 exactly as described above. The values from wells coated just with BSA and antibody were subtracted from each well treated with the same antibody. Results are reported as % inhibition as Optical Density (570 nm) with antibody treated sample/no antibody control×100 (n=3).

FIG. 2: Example of a single particle cryoelectron microscopy map of the Fab ADWA11 2.4 in complex with the ectodomain of human αvβ8 at overall resolution of 4.1 Å. A) Shown inside the map is a ribbon model. B) Shown is a close-up the cryoEM density map with ribbon model and C) a ribbon model showing interactions of the ADWA11 2.4 Fab with αvβ8. D) The ADWA 11 2.4 epitope. Shown is the ADWA 11 2.4 footprint on the αvβ8 head domain enface (in white) with the epitope on αv-subunit (mesh) and β8 subunit (grey). E) The same orientation of the αvβ8 head with the footprint of C6D4 (PDB: 6UJB) with the epitope on αv-subunit (mesh) and β8 subunit (grey). Results show that ADWA 11 2.4 is centered more on the αv-head than C6D4 which binds extensively to the β8 subunit. Compared to the structure of αvβ8 in complex with TGF-β (6UJA), ADWA-11 2.4 only partially occludes the binding pocket while C6D4 more completely blocks the binding pocket. F) Shown is the density map in the same orientation as in E of the αvβ8 head with the footprint of ADWA 11 2.4 compared to L-TGF-β1 (6UJA). The area covered by each footprint is shown (av epitope in wire mesh, β8 epitope in black). The footprints of TGF-β (920.7 Å², 6UJA), and ADWA-11 2.4 (998.7 Å²) have minimal overall overlap (0% of αv interactions in common and 8% of β8 subunit interactions in common). These data indicate that the mechanism of ADWA 11 2.4 is either steric or allosteric or a combination of both, rather than a direct competitive inhibitor.

FIG. 3: Binding interactions of ADWA 11 2.4/αvβ8. The interactions between ADWA 11 2.4 (variable domain heavy chain) VH and (variable domain light chain) VL with integrin αvβ8 as determined by the cryoEM structure of ADWA11 2.4 with αvβ8 (dotted lines). Framework (Fr) and complementary defining regions (CDR) are designated in the Kabat convention. Grey boxed residues interact with the αv- or β8-subunit in the cryo-EM structure. The amino acid number designations are shown for integrin β8 subunit (SEQ ID NO:52) and the integrin αv subunit (SEQ ID NO:53). Data shows extensive differences in epitope interactions compared with HuF12 and H4C8.

FIG. 4: Example of a single particle cryoelectron microscopy map of the Fab F12 in complex with the ectodomain of human αvβ8 at local resolution of 3.1 Å (A). Shown inside the map is a ribbon model. B) Shown is a plot of the FSC curve vs resolution.

FIG. 5: Cryo-EM of the αvβ8/HuF12 Fab complex. A) Shown is the cryo-EM density map and B) model showing the interactions of the F12 Fab with αvβ8. C) The HuC6D4 footprint is shown as a comparison to D) HuF12. The αvβ8 head domain enface (in white) with the epitope on αv-subunit (mesh) and β8 subunit (grey). Results show that the footprint of the HuF12 is slightly increased in size (1420.2 Å²) compared to HuC6D4 (1,306.7 Å²).

FIG. 6: The interactions between huC6D4F12 VH and VL with integrin αvβ8 as determined by the cryo-EM structure of huC6D4F12 with αvβ8. Framework (Fr) and complementary defining regions (CDR) are designated in the Kabat convention. Interacting residues (underlined) between the VH and VL with the αv-subunit (SEQ ID NO:53) or β8-subunit (SEQ ID NO: 52) in the cryo-EM structure are indicated with dotted lines.

FIG. 7: Combining the mutations D31R in CDR1 VH and N31H in CDR1 VL are key for improved function of huF12 Vh. The VH of F12 (SEQ ID NO: 24) was combined either with the huC6D4 VL (SEQ ID NO: 23) or with the huF12 VL (SEQ ID NO: 25). The huC6D4 VH (SEQ ID NO: 22 was combined with the huF12 VL (SEQ ID NO: 25) or with the huC6D4 VL (SEQ ID NO: 23). The antibodies were expressed as murine IgG2a by fusing the variable regions with the murine CH1-CH3 (SEQ ID NO: 60). The purified antibodies were applied to wells coated with integrin αvβ8 together with TMLC GAPR/TGF-β1 expressing reporter cells. Shown is the activation percentage relative to isotype SV5 treated wells. The concentration of inhibitor required to reduce the signal by 50% (IC50) was determined (PRISM) and shown for each antibody (μg/ml) below the figure.

FIG. 8: HuF12 with improved affinity shows dose-related anti-tumor activity. A-B) Lewis lung carcinoma cells (LLC) were either transfected with empty expression plasmid (mock) or β8 expression plasmid. The transfected cells were selected and sorted for high uniform expression of β8 (β8 LLC). C57Bl/6 mice were injected with on one flank with A) mock and on the opposite with B) 38 LLC cells. Day 4 after tumor cell injection, tumors were palpable and the mice were randomized and injected with either SV5 (isotype IgG2a control, 10 mg/kg IP), or HuF12-IgG2a (either at 1, 3, 7 or 10 mg/kg, I.P.). N=at least 5 mice/group. After 14 days tumors were weighed. Shown is ANOVA with p test for trend. ****p<0.0001

FIG. 9: Improving the affinity of C6D4 improves anti-tumor activity. A-B) Lewis lung carcinoma cells (LLC) were either transfected with empty expression plasmid (mock) or β8 expression plasmid. The transfected cells were selected and sorted for high uniform expression of β8 (β8 LLC). C57Bl/6 mice were injected with on one flank with A) mock and on the opposite with B) β8 LLC cells. Day 4 after tumor cell injection, tumors were palpable and the mice were randomized and injected with either SV5 (isotype IgG2a control), HuC6D4-IgG2a, or HuF12-IgG2a (10 mg/kg, I.P.). Injections were repeated again on day 11, and tumors harvested on day 14. Shown are the mass of individual tumors. The experiment was repeated 3 times with 10 mice/group for each individual experiment. One way ANOVA followed by Tukey's post-test was performed and p values shown.

FIG. 10: High affinity anti-αvβ8 antibodies to the C6D4 epitope are more effective at inhibiting αvβ8-mediated TGF-activation that other anti-avb8 antibodies. A) HuF12 or ADWA 11 2.4 were expressed as IgG2a and applied to wells coated with the ectodomain of αvβ8 (1 μg/ml coating concentration) in the presence of TMLC GARP/TGF-β1 cells. Shown is a dose response representative experiment with TGF-β activation (luciferase units) shown relative to isotype control antibody over the indicated concentrations. B) HuF12, MHG-8 (SEQ ID NOs: 84, 85), SRK181 (Ab6) (SEQ ID NO: 64, 65) were expressed as IgG2a and applied to wells coated with the ectodomain of αvβ8 (1 μg/ml coating concentration) in the presence of TMLC GARP/TGF-β1 cells. Shown is a dose response representative experiment with TGF-β activation (luciferase units) relative to isotype control antibody over the indicated concentrations. C) C57B/6 mice were injected on one flank with β8 LLC tumor cells and on the opposite flank, mock transfected LLC cells. After tumor establishment, 4 days post tumor cell injection, the indicated antibodies were injected IP (10 mg/kg I.P.). SV5 is the murine IgG2a isotype control and the remaining antibodies all made with human CH1 and murine IgG2a hinge and Fc regions: ADWA11 2.4 (from Pat no. US2020/0079855 A1), HuC6D4 (Takasaka, et al (2018), JCI Insight), HuF12, or anti-LAP, HuSRK-181 (Ab6: U.S. Pat. No. 10,751,413).

FIG. 11: A highly humanized prototype of HuC6D4F12 (H1.1) is designed using a cryo-EM structure of HuC6D4F12 with integrin αvβ8. HuC6D4F12 residues which contact αvβ8 are preserved. As an intact human IgG4, H1.1 has ˜70 fold decrease in affinity compared to HuC6D4F12. Humanization is based on the closest IMGT human sequences (IGHV1-24_IGHD5-18_IGHJ6, SEQ ID NO: 29; IGKV3-15_IGKJ1, SEQ ID NO: 33) and rational design based on the huF12/αvβ8 cryo-EM structure. The human amino acids in the IMGT sequences which differ are shown above the HuC6D4F12 sequences. Framework (Fr) and complementary defining regions (CDR) are designated in the Kabat convention. Underlined residues interact with αvβ8 in the cryo-EM structure.

FIG. 12: Design of yeast display libraries. Several yeast display libraries (Lib-1-3) were generated using slice-overlap extension PCR and degenerate positions coding for the indicated amino acid changes. Degenerate positions in the library coded for human (Vh: IGHV1-24_IGHD5-18_IGHJ6 (SEQ ID NO29; VL: IGKV3-15_IGKJ1 (SEQ ID NO: 33) or mouse residues (underlined) or introduced new amino acids, which were rationally designed based on the cryo-EM structure of HuF12 with αvβ8 shown below the lib-1-3 mutations. The scFV yeast display libraries were ligated in-frame into the scFV vector pYD4 and transformed into yeast using gap-repair, as described (see Takasaka, et al (2018) JCI Insight 3; 20). CDRs and Fr are listed in the Kabat convention.

FIG. 13: Initial yeast library approach to improve affinity of Clone H1.1. A-B) After induction and screening of the pooled yeast transformants for scFV expression using anti-V5 (clone SV5), the αvβ8 ectodomain was incubated with the yeast pool, washed and scFV which bound to αvβ8 were detected using anti-human αv (clone 8B8). After several rounds of selection using increasing stringency, individual clones were picked from the parental library pool, and the pool enriched for binders and clones were individually sequenced. Presence of the various amino acids coded for by degenerate codons were verified in sequences of individual clones from the presorted libraries. A) Shown are clones with improved binding with changes in amino acids that were over-represented in sequenced clones. Sequences are shown in the Kabat convention. B) Representation of scFV binding affinity to the αvβ8 ectodomain. The binding affinities were determined using a least-squares fit model based on the hyperbolic equation y=m1+m2*m0/(m3+m0), where y=MFI at different avb8 concentrations; m0=αvβ8 concentration; m1=MFI with no αvβ8; m2=MFI at saturation; and m3=KD.

FIG. 14: Effect of humanized VH Framework-1 and VL CDR-1 on F12 binding. Clones with a humanized Fr1 with either HuC6D4F12 Vh CDR-1, or the VH CDR-1 from clones B4, A5, A10 or A12 were created by PCR, paired with the huC6D4F12 VL, and transformed into yeast into pYD4 using gap-repair, and sequences from individual clones verified, as described (see Takasaka, et al. JCI Insight (2018) 3; 20). After induction the transformants were assessed for scFV expression using anti-V5 (clone SV5). The αvβ8 ectodomain was incubated with the transformed yeast clones, washed and binding detected using anti-human αv (clone 8β8). Presence of the various amino acids coded for by degenerate codons were verified in sequences of individual clones. A) The sequence of HuC6D4F12 is shown with amino acids that are different in Clone H1.1 shown above. The different amino acids changed to human in HuC6D4F1 (HuFr1 Vh), are shown immediately below the huC6D4F12 Vh sequence. Below are the different amino acid sequences of B4, A5, A10 and A12 from HuC6D4F12 Vh. The HuC6D4F12 VL sequence was paired with the indicated VH sequences. The different amino acids in clone H1.1 are shown above. B) The binding affinities of clones were determined using a least-squares fit model based on the hyperbolic equation y=m1+m2*m0/(m3+m0), where y=MFI at different αvβ8 concentrations; m0=αvβ8 concentration; m1=MFI with no αvβ8; m2=MFI at saturation; and m3=KD. Note that the 6 amino acid changes in clone A5/F12 resulted in non-specific binding and binding affinity was not possible to calculate (shown as dotted bar with arbitrary affinity).

FIG. 15: Identification of humanized VL variants which preserve binding when paired with HuC6D4F12 VH. A) The VH from wild-type HuC6D4F12 (SEQ ID NO:24) was paired with the VL from clones A5 (SEQ ID NO:71) and A12 (SEQ ID NO: 76), ligated in frame into the scFV vector pYD4 and transformed into yeast using gap-repair, as described (see Takasaka, et al. JCI Insight (2018) 3; 20). After induction and screening of individual clones for scFV expression using anti-V5 (clone SV5), the αvβ8 ectodomain was incubated with the yeast pool, washed and scFV which bound to αvβ8 were detected using anti-human αv (clone 8β8). B) The scFV binding affinities of the VH HuF12 paired with A5 VL or A12 VH were determined using a least-squares fit model based on the hyperbolic equation y=m1+m2*m0/(m3+m0), where y=MFI at different αvβ8 concentrations; m0=αvβ8 concentration; m1=MFI with no αvβ8; m2=MFI at saturation; and m3=KD. The underlined amino acid (S25) in CDR1 in panel A is the key mouse residue present in A5 but not A12 required for maintenance of affinity.

FIG. 16: Affinity optimization of humanized F12 variants. A) A scFV yeast display library (Library 3) was created with the indicated degenerate positions coding for the indicated novel amino acids (i.e. not in germline human or mouse Ig sequence databases), back mutations to mouse from clone H1.1 (shown as underlined residues), as well as final humanization of Fr4 VH and VL. Libraries were ligated in frame into the scFV vector pYD4 and transformed into yeast using gap-repair, as described (see Takasaka, et al. JCI Insight (2018) 3; 20). After induction and screening of the pooled transformants for scFV expression using anti-V5 (clone SV5), the avb8 ectodomain was incubated with the yeast pool, washed and scFV which bound to αvβ8 were detected using anti-human αv (clone 8β8). After multiple rounds of selection by sorting yeast stained under conditions of increasing stringency (Low Ag concentration, short Kon and long Koff), individual clones were isolated, sequenced and tested for binding affinity. Shown are the sequences of VH and VL pairs enriched in the library. The binding affinities of representative yeast clones was determined using a least-squares fit model based on the hyperbolic equation y=m1+m2*m0/(m3+m0), where y=MFI at different αvβ8 concentrations; m0=αvβ8 concentration; m1=MFI with no αvβ8; m2=MFI at saturation; and m3=KD. Shown are novel amino acids introduced into the Vh CDR domains. The calculated binding affinities of select yeast scFV clones was HuF12: 6.7 nM; H4C8: 3.8 nM; H4G9: 8.6 nM; H4G8: 8.9 nM; H4C10: 10.1 nM. Also shown are new humanized amino acids introduced into the parental HuC6D4F12 clone either in H.1.1 and/or in degenerate positions in derivative libraries. Note that binding for clone H1.1 as scFV on yeast was too low to be measurable. Underlined residues represent amino acids that were back-mutated to be identical in HuC6D4F12.

FIG. 17: Characterization of cell surface binding of humanized affinity optimized anti-αvβ8 antibodies. The cell surface binding affinities of H4C8, H4G9, H4C10, H4G8 compared to HuC6D4F12 expressed as IgG4 (S228P to prevent arm exchange, R409K and L445P to reduce aggregation and to promote stability). CHO cells stably transfected and sorted for uniform human αvβ8 expression were incubated with various antibodies at indicated concentrations for 15 mins in autoMACS separation buffer (Miltenyi Biotec (130-091-221)), after washing thrice in autoMACS buffer, staining was detected using fluorophore conjugated secondary antibodies (Jackson Immunoresearch Goat anti-mouse H+L PE conjugate (115-116-146), Jackson Immunoresearch Goat anti-human H+L PE conjugate (109-116-088)). Cell surface affinity was determined using the least-squares fit model based on the hyperbolic equation y=m1+m2*m0/(m3+m0), where y=MFI at different avb8 concentrations; m0=αvβ8 concentration; m1=MFI with no αvβ8; m2=MFI at saturation; and m3=KD.

FIG. 18: Binding characteristics of H4C8 of formalin fixed αvβ8 compared to HuC6D4F12 or ADWA11 2.4 (from Pat no. US2020/0079855 A1). CHO αvβ8 expressing cells were fixed in 2% buffered formalin in PBS for 15 min at RT and then stained at the indicated concentrations as described in FIG. 17. The binding affinities to unfixed or formalin fixed cells is shown. Cell surface affinity was determined using the least-squares fit model based on the hyperbolic equation y=m1+m2*m0/(m3+m0), where y=MFI at different αvβ8 concentrations; m0=αvβ8 concentration; m1=MFI with no αvβ8; m2=MFI at saturation; and m3=KD.

FIG. 19: Solution-based affinity of H4C8 is 7.07 pM as measured by KinExA (Sapidyne Instruments, Inc., Boise, ID), a flow fluorimeter which allows true measurement of binding affinity and kinetics for unmodified molecules in solution, including antibody-antigen interactions. Samples with serial dilutions are drawn over solid-phase beads coupled with one binding partner to capture the complimentary binding molecule, which is detected by flow of fluorophore-labeled antibody solution binding to a separate epitope on the same molecule being measured. Signals are used to calculate free concentration in solution after equilibrium is reached (i.e. to determine binding affinity KD), or under pre-equilibrium conditions over time (i.e. to determine binding kinetics). Free antigen remaining in solution at equilibrium for varying initial antibody concentrations in constant initial concentration of antigen as determined by the KinExA software, based on optimized least-squares fitting to a reversible binding equation.

FIG. 20: H4C8 maintains ability to block αvβ8-mediated TGF-β activation similar to HuC6D4F12 while maintaining specificity for αvβ8. A) Shown are inhibition curves for the indicated antibody abilities to block the ability of immobilized human αvβ8 to activate human L-TGF-β presented on the cell surface of TMLC by GARP. The assays were performed exactly as described in (Seed et al, Sci Immunol, 2021 and Campbell, et al, Cell 2019). The estimated ED50 values are shown. The antibodies used were HuC6D4 IgG2a, HuC6D4F12 IgG2a or H3.H12, H4C8 or H4C8m (murine VL Fr4) as human IgG4. B) The specificity of H4C8 was tested against immobilized ectodomains of αvβ1, αvβ3, αvβ5, αvβ6 and αvβ8 (either purchased from R &D systems or produced in ExpiCHO cells. All ectodomains were plated at 1 μg/ml and after washing, wells were blocked with 1% BSA. The indicated antibodies (either human IgG4 isotype or H4C8 IgG4).

FIG. 21: Cryo-EM of H4C8 in complex with αvβ8. A) Example of a single particle cryo-EM map of the Fab H4C8 in complex with the ectodomain of human αvβ8 at local resolution of 3.19 Å. Shown inside the map is a ribbon model. B) Shown on the right is a plot of the FSC curve vs resolution.

FIG. 22: Binding interface between H4C8 and αvβ8. A) Shown is the cryo-EM density map with the H4C8 Fab on top interacting with the αv-head (left) and β8 head (right) and B) model showing the interactions of the H4C8 Fab with αvβ8 in similar orientation as in A. Dotted lines indicate interactions shown in Table VI. C) Shown is the density map in the same orientation as in 2F, and 5C, D with the αvβ8 head with the footprint of L-TGF-β1 (6UJA) compared with HuF12 and H4C8 Fabs. The area covered by each footprint is shown (av epitope in wire mesh, β8 epitope in black). The footprints of TGF-β (6UJA), and F12 and H4C8 have extensive overlap (H4C8: 100% of αv interactions in common and 75% of β8 subunit interactions in common; HuF12: 80% of αv interactions in common and 71% of β8 subunit interactions in common). These data indicate that the mechanisms of H4C8 and HuF12 are either direct competition or steric inhibition, or both combined.

FIG. 23. The VH Fr3 L72Q mutation increases interaction interface between H4C8 VH CDR1 and CDR2 with β8. A-B) Close-up of ribbon model including the A) H4C8 and B) HuF12 binding interface between Vh CDR1 and CDR2 with the integrin β8 subunit. Shown are dotted lines between indicated interacting amino acids. Q72 and L72 in VH Fr3 of H4C8 (A) and HuF12 (B) are indicated. Data shows that VH Fr3 L72Q mutation is a unique feature of H4C8 and related antibodies.

FIG. 24: The interactions between H4C8 VH and VL with integrin αvβ8 as determined by the cryo-EM structure of H4C8 with αvβ8. Framework (Fr) and complementary defining regions (CDR) are designated in the Kabat convention. Underlined residues interact with the αv- or β8-subunit in the cryo-EM structure, as illustrated with dotted lines.

FIG. 25: Effector function is not required for anti-b8 inhibition of tumor growth. A-J) C57B/6 mice were injected on one flank with Lewis lung carcinoma cells stably transfected with mouse itgb8 and sorted to uniform surface expression (B, D, F, H, J). On the contralateral flank mock transfected LLC cells which naturally express low level of αvβ8, were injected (A, C, E, G, I). Day 5 after injection, established tumors were measured, and mice injected with the indicated antibodies (10 mg/kg I.P.) and again on day 12 (arrows). Investigators were blinded to both β8 expression and the antibody treatment. A) anti-SV5, IgG2a, C-D) HuF12 IgG2a, E-F) mouse IgG1 isotype control, G-H) HuF12 as mouse IgG1, I-J) HuF12 as mouse IgG1 D265A. Tumor growth was measured with digital calipers. The SV5 antibody is the control for HuC6D4F12 (HuF12). mIgG1 isotype control was purchased from (BioXcell, MOPC-21). N=10/treatment condition. Data provides rationale for reduced effector function in anti-αvβ8 humanized antibodies.

FIG. 26: Effector function is not required for anti-β8 inhibition of tumor growth. Mouse IgG2a has strong effector function while mouse IgG1 has weak effector function similar to human IgG4. The effector function of mouse IgG1 can further be reduced by the D265A mutation. C57B/6 mice were injected on one flank with Lewis lung carcinoma cells stably transfected with mouse itgb8 and sorted to uniform surface expression. On the contralateral flank mock transfected LLC cells which naturally express low level of αvβ8, were injected. Day 5 after injections tumors were established and mice injected with the indicated antibodies (10 mg/kg I.P.) and again on day 12. Investigators were blinded to both 38 expression and the antibody treatment. Tumor growth was measured with digital calipers. The SV5 antibody is IgG2a and a control for HuC6D4F12 (HuF12). mIgG1 isotype control was purchased from (BioXcell, MOPC-21). *p<0.05 by one-way ANOVA and Tukey's post-test to find where the differences lay. N=10/treatment condition. Mouse IgG1 has limited effector function considered to be similar to IgG4 in humans. IgG2a in mouse has maximal effector function similar as IgG1 in humans. Data provides rationale for reduced effector function in anti-αvβ8 humanized antibodies, but also suggests that some effector function should be preserved for maximal therapeutic benefit.

FIG. 27A-D: Lower β8 expression decreases MC38 tumor growth and improves complete tumor regression using antibodies directed to the HuC6D4/HuF12/H4C8 epitope. A) MC38 cells were transduced with lentiviral siRNA constructs, control siRNA (csiRNA) or itgb8 siRNA (itgb8 siRNA). Transduced itgb8siRNA pools were sorted for low expression of αvβ8 and resulting pools were stained for surface αvβ8 expression using HuF12 followed by a secondary anti-mouse PE. Isotype control indicates pools of itgb8 siRNA treated and sorted cells stained with anti-SV5 (IgG2a). B) MC38 tumors from pools of itgb8siRNA or csiRNA cells were established on opposite flanks of C57B/6 mice, and mice treated 5 and 11 days post cell inoculation with isotype B) were compared with mice treated with C,D) HuC6D4 or HuF12 (all 10 mg/kg I.P.). Growth curves of MC38 tumors from pools of itgb8siRNA (C) vs csiRNA cells (D).*p<0.05, **p<0.001, ***p<0.001, ****p<0.0001 by Student's t-test for comparison of two groups or two-way ANOVA to compare effects of two treatments (itgb8siRNA) and inhibitory monoclonal antibodies. Tukey's post-test is used to find where the differences lay. N=10/treatment condition. Data shows that tumor cell expression of itgb8 increases tumorigenicity and that tumors with lower itgb8 expression are more responsive to anti-αvβ8. E-J) Spider plots of MC38 tumor growth, E, G, I, control siRNA (csiRNA) or F, H, J itgb8 siRNA (itgb8 siRNA) treated with isotype E, F) compared with mice treated with G, H) HuC6D4 or I, J) HuF12. N=10/treatment condition. Complete response (CR) or partial responses (PR) are shown. CR is defined is no palpable tumor, PR as tumor growth endpoint reached after day 24 when all mice treated with isotype control anti-SV5 reached size endpoint. Data shows that reducing αvβ8 expression in tumor cells increases effectiveness of anti-tumor effects of anti-αvβ8. Data also shows that increasing affinity of anti-αvβ8 antibodies improves effectiveness of anti-tumor responses and as monotherapy can completely inhibit tumor growth of tumors with low expression of αvβ8. K: Antibody affinity improves in vivo performance: monotherapy with HuF12 improves survival compared with isotype or HuC6D4 treatment. Shown are survival curves of mice with differences in survival between groups assessed using the Mantel-Cox test. *p<0.05, **p<0.001, ***p<0.001. Shows that increased affinity of anti-αvβ8 antibodies correlates with improved anti-tumor response.

FIG. 28 HuF12 treatment shows improved tumor response in MC38 model in combination with anti-PD1. A-H) Spider plots of tumor growth established from pools of sorted MC38 cells transduced with A, C, E, G, itgb8 siRNA vs B, D, F, H, control siRNA (csiRNA). Antibodies (SV5, HuC6D4, HuF12, or anti-PD1 (RMPI-14, BioXCell) were administered (10 mg/kg I.P.) on days indicated by upwards dotted arrows (SV5, HuC6D4 or HuF12) or downwards arrows (anti-PD1). N=10/treatment condition. Complete response (CR) or partial responses (PR) are shown. CR is defined is no palpable tumor, PR as tumor growth endpoint reached after day 24 when all mice treated with isotype control, HuC6D4, or HuF12 anti-SV5 reached size endpoint. I-L: HuF12 treatment improves tumor response in MC38 model in combination with anti-PD1. itgb8 knock-down increases response to anti-PD1. I-L) Plots of average tumor growth established from pools of sorted MC38 cells transduced with itgb8 siRNA vs control siRNA (csiRNA) in combination with antibodies (SV5, HuC6D4, HuF12, or anti-PD1 (RMPI-14, BioXCell). Antibodies were administered (10 mg/kg I.P.) on days indicated by downwards dotted arrows (SV5, HuC6D4 or HuF12) or downwards arrows (anti-PD1).*p<0.05, **p<0.001, ***p<0.001, ****p<0.0001 by Student's t-test for comparison of two groups or two-way ANOVA to compare effects of two treatments (itgb8siRNA) and inhibitory monoclonal antibodies. Tukey's post-test is used to find where the differences lay. N=10/treatment condition. Data shows that inhibiting αvβ8 with a high affinity αvβ8 antibody increases the effectiveness of checkpoint inhibitors.

FIG. 29: HuF 12 treatment in combination with anti-PD1 produces durable anti-tumor response. A-B) Mice with complete responses to various treatments (anti-PD1 (PD1), anti-HuC6D4+anti-PD1, or anti-HuF12+anti-PD1) were injected with the indicated MC38 cells on opposite flanks A) itgb8 siRNA or B) csiRNA and followed for tumor growth. Treatment naive mice (control) previously treated with isotype control (SV5) were also injected with MC38 cells on opposing flanks. Plots of average tumor growth established from pools of sorted MC38 cells transduced with itgb8 siRNA vs control siRNA (csiRNA) are shown. Mice did not receive any additional antibody treatment. *p<0.05, **p<0.001, ***p<0.001, ****p<0.0001, one-way ANOVA used to compare effects of different treatments regimens on tumor rechallenge. Tukey's post-test is used to find where the differences lay. Numbers of mice in each group are indicated in the figure. Data indicates antibodies with high affinity for αvβ8 can produce durable anti-cancer immune responses in combination with checkpoint inhibitors.

FIG. 30: Cell surface binding to αvβ8 and αvβ6 of H4C8 RGD3, H4C8 RGD1, H3C8 RGD3 compared to HuC6D4 F12. All antibodies were in IgG4 format (SEQ ID NO: 59). A) CHO cells expressing human αvβ8 or B) αvβ6 were stained with the indicated antibodies.

FIG. 31: Antibody binding to αvβ8 and αvβ6 expressing CHO cells at a non-saturating dose (1 μg/ml) of H3C8 RGD3, H4C8 RGD1, H4C8 RGD3 compared to HuC6D4 F12. All antibodies were in IgG4 format (SEQ ID NO: 59). CHO cells expressing human αvβ8 or αvβ6 were stained with the indicated antibodies at 1 μg/ml and detected as described in FIG. 17.

FIG. 32: H4C8 RGD3 and H4C8 RGD1 have improved ability to block both αvβ8 and αvβ6-mediated TGF-β activation. A-B) Shown are inhibition curves for the indicated antibody abilities to block immobilized human A) αvβ8 tor B) αvβ6 activation of human L-TGF-β presented on the cell surface of TMLC by GARP. The assays were performed exactly as described in (Seed, et al (2021) Sci Immunol, 2021 and Campbell, et al (2019) Cell). The estimated ED50 values are shown (μg/ml). F12 RGD1 did not significantly inhibit activation and is not shown. H3C8 RGD3, H4C8 RGD1, H4C8 RGD3 compared to HuC6D4 F12. All antibodies were in human IgG4 (SEQID NO: 59) format except RGD3 and 3G9 which were in mouse IgG2a. RGD3 consists of HuC6D4 VH combined with HuC6D4 with the RGD3 (GRGDLGRLKK) grafted in VL CDR1. Shown is the calculated percent inhibition for each antibody over multiple assays (n>3). Antibodies with no inhibition are indicated by the infinity symbol.

FIG. 33 H4C8 RGD3 and H4C8 RGD1 show specificity for αvβ6 and αvβ8, VL Fr4 K108/V109 humanization improves or maintains binding, VH Fr4 Q72 improves binding over E72. A) Binding of anti-β3 (AP3) relative to F12 IgG4, H4C8 RGD1 IgG4,H4C8 RGD3 IgG4, H3C8 RGD3 with VL Fr4 V108/L109 (mouse) IgG4, H4C8 RGD3 IgG4, H3C8 RGD3 VL Fr4 K108/V109 (human) IgG4, H4C8 RGD1 VH Fr3 E72, H4C8 RGD3 VH Fr3 E72 IgG4. HEK 293 cells stably expressing αvβ3 were stained and the percentage of relative binding to the mean fluorescence intensity of AP3 is shown, B) Expi293 cells transiently expressing αvβ6 were stained and the percentage of relative binding to the mean fluorescence intensity of 3G9 is shown, C) HEK 293 cells stably expressing αvβ8 were stained and the percentage of relative binding to the mean fluorescence intensity of HuF12 is shown. All primary antibodies were used at 2.5 ug/ml. D) The above antibodies (F12 IgG4, H4C8 RGD1 IgG4,H4C8 RGD3 IgG4, H3C8 RGD3 with VI Fr4 V108/L109 (mouse) IgG4, H4C8 RGD3 IgG4, H3C8 RGD3 VI Fr4 K108/V109 (human) IgG4, H4C8 RGD1 Vh Fr3 E72, H4C8 RGD3 Vh Fr3 E72 IgG4) do not bind to the immobilized ectodomains of αvβ1 and αvβ5. αvβ1 and αvβ5 (R&D Systems), and αvβ6 ectodomains were immobilized, blocked (BSA) and allowed to bind to primary antibodies. Binding or 3G9 is shown and percent binding of antibodies relative to 3G9 binding to αvβ6 is shown. Dotted line indicate binding observed with each positive control antibody,

FIG. 34: H4C8 RGD1 and H4C8 RGD3 bind to mouse αvβ6 and αvβ8. A) Expi293 cells were transiently transfected with mouse β6 and 3 days after transfection were stained with HuF12, 3G9, RGD3, F12 RGD1, H4C8 RGD3 and H4C8 RGD1. Shown is the Mean Fluorescence intensity of binding of the antibodies. The non-transfected Expi393 cells were stained in parallel and showed no background staining. B) 38 LLC and mock LLC (Control) cells were stained with the same antibodies. Shown is the Mean Fluorescence intensity (MFI) of binding of the antibodies. Results show that H4C8 RGD3 and H4C8 RGD1 both bind to mouse αvβ6 and mouse αvβ8.

FIG. 35: Multi-dimensional immune profiling identifies ITGB8 as potential marker for immunotherapy resistant tumor types A) An unsupervised network clustering approach based on 10 tumor specific immune gene signatures, from transcriptomic data derived from sorted live tumor and immune cells populations from 364 tumor types (Bladder, Colorectal, Glioblastoma, Gynecologic, Liver, Head and Neck, Kidney, Lung, Skin, Pancreas, Sarcoma) yielded 12 unique “archetypes”. B, C) The tumor archetypes span tumor types. Archetype gene signatures were generated by differential gene expression analysis, between the live compartments. Code: BLAD=bladder carcinoma, CRC=colorectal carcinoma, GBM=glioblastoma, GYN=gynecologic, HEP=Hepatoma, NHSC=head and neck squamous cell, KID=kidney cancer, LUNG=non-small cell lung cancer, MEL=melanoma, PDAC=pancreatic ductal adenocarcinoma, PNET=primitive neuroectodermal tumor, SRC=sarcoma. D) The median TPM expression per archetype of each of the genes in the gene signatures were clustered and used to identify the same tumor archetypes in a TCGA dataset 4000 tumor of various types and stage. E) In CD8+ T cells biased archetypes, the Immune Desert Macrophage bias archetype 12 has significantly worse outcome than immune rich archetypes-1 and -2. F) Using multivariate analysis across tumors there were significant differences in outcome between archetypes with similar T cell subset enrichment regardless of the tissue of origin. In CD8+ T cells biased archetypes, the Immune Desert Macrophage bias archetype 12 has significantly worse outcome than immune rich archetypes-1 and -2. G) Using hierarchical clustering of the median integrin subunit expression from RNA from isolated tumor cells within each archetype, ITGB8 stood out from all other integrins as being highly expressed in the Immune Desert Macrophage bias archetype 12. This archetype contains melanoma, gynecologic, head and neck carcinoma, and sarcomas. The differentially expressed genes are shown as a heat map with the lighter the bar, the higher the expression. The position of ITGB8 is indicated by an arrow.

FIG. 36 High integrin αvβ8 expression predicts non-responsiveness to checkpoint inhibition. Box and whiskers plot representing tumor samples from patients with non-small cell carcinoma that were treated with front-line checkpoint inhibitor (monotherapy) stained for integrin αvβ8 using clone F9 (Takasaka, et al. JCI Insight (2018) 3; 20). The patients were categorized as non-responders (grey bars, n=6) versus partial responders (open bars, n=14). Antibody staining was characterized as positive with membrane staining, or negative by the absence of staining. Immunohistochemical staining was performed using the Leica (Bond-III) automated staining platform. Tumor proportion scoring (TPS) was performed and interpreted as described for PD-L1 staining using clone 22C3 (www.agilent.com). **p=0.0039, Student's t test, two-tailed.

### DEFINITIONS

Unless defined otherwise, technical and scientific terms used herein have the same meaning as commonly understood by a person of ordinary skill in the art. See, e.g., Lackie, DICTIONARY OF CELL AND MOLECULAR BIOLOGY, Elsevier (4^{th} ed. 2007); Sambrook et al., MOLECULAR CLONING, A LABORATORY MANUAL, Cold Springs Harbor Press (Cold Springs Harbor, NY 1989). Any methods, devices and materials similar or equivalent to those described herein can be used in the practice of this invention. The following definitions are provided to facilitate understanding of certain terms used frequently herein and are not meant to limit the scope of the present disclosure.

The terms “anti-αvβ8 antibody,” “αvβ8 specific antibody,” “αvβ8 antibody,” and “anti-αvβ8” are used synonymously herein to refer to an antibody that specifically binds to αvβ8. Similarly, an anti-β8 antibody (and like terms) refer to an antibody that specifically binds to β8. The anti-αvβ8 antibodies and anti-β8 antibodies described herein bind to the protein expressed on αvβ8 expressing cells. Similar conventions apply for antibodies that bind to αvβ6 or other integrins. The terms “αvβ8” and “avb8” are used synonymously and refer to human integrin αvβ8 unless specifically indicated otherwise (for example if indicated as originating in a mouse).

An αvβ8-associated disorder is a condition characterized by the presence of αvβ8-expressing cells, either cells expressing an increased level of αvβ8, or increased number of αvβ8-expressing cells relative to a normal, non-diseased control. TGFβ-associated disorders (disorders characterized by higher than normal TGFβ activity) include αvβ8-associated disorders, as αvβ8 is involved in activating TGFβ in certain circumstances, as described herein.

“Nucleic acid” refers to deoxyribonucleotides or ribonucleotides and polymers thereof in either single- or double-stranded form, and complements thereof. The term “polynucleotide” refers to a linear sequence of nucleotides. The term “nucleotide” typically refers to a single unit of a polynucleotide, i.e., a monomer. Nucleotides can be ribonucleotides, deoxyribonucleotides, or modified versions thereof. Examples of polynucleotides contemplated herein include single and double stranded DNA, single and double stranded RNA, and hybrid molecules having mixtures of single and double stranded DNA and RNA.

The words “complementary” or “complementarity” refer to the ability of a nucleic acid in a polynucleotide to form a base pair with another nucleic acid in a second polynucleotide. For example, the sequence A-G-T is complementary to the sequence T-C-A. Complementarity may be partial, in which only some of the nucleic acids match according to base pairing, or complete, where all the nucleic acids match according to base pairing.

The words “protein”, “peptide”, and “polypeptide” are used interchangeably to denote an amino acid polymer or a set of two or more interacting or bound amino acid polymers. The terms apply to amino acid polymers in which one or more amino acid residue is an artificial chemical mimetic of a corresponding naturally occurring amino acid, as well as to naturally occurring amino acid polymers, those containing modified residues, and non-naturally occurring amino acid polymer.

The term “amino acid” refers to naturally occurring and synthetic amino acids, as well as amino acid analogs and amino acid mimetics that function similarly to the naturally occurring amino acids. Naturally occurring amino acids are those encoded by the genetic code, as well as those amino acids that are later modified, e.g., hydroxyproline, γ-carboxyglutamate, and O-phosphoserine. Amino acid analogs refers to compounds that have the same basic chemical structure as a naturally occurring amino acid, e.g., an a carbon that is bound to a hydrogen, a carboxyl group, an amino group, and an R group, e.g., homoserine, norleucine, methionine sulfoxide, methionine methyl sulfonium. Such analogs may have modified R groups (e.g., norleucine) or modified peptide backbones, but retain the same basic chemical structure as a naturally occurring amino acid. Amino acid mimetics refers to chemical compounds that have a structure that is different from the general chemical structure of an amino acid, but that functions similarly to a naturally occurring amino acid.

Amino acids may be referred to herein by either their commonly known three letter symbols or by the one-letter symbols recommended by the IUPAC-IUB Biochemical Nomenclature Commission. Nucleotides, likewise, may be referred to by their commonly accepted single-letter codes.

“Conservatively modified variants” applies to both amino acid and nucleic acid sequences. With respect to particular nucleic acid sequences, conservatively modified variants refers to those nucleic acids which encode identical or essentially identical amino acid sequences, or where the nucleic acid does not encode an amino acid sequence, to essentially identical or associated, e.g., naturally contiguous, sequences. Because of the degeneracy of the genetic code, a large number of functionally identical nucleic acids encode most proteins. For instance, the codons GCA, GCC, GCG and GCU all encode the amino acid alanine. Thus, at every position where an alanine is specified by a codon, the codon can be altered to another of the corresponding codons described without altering the encoded polypeptide. Such nucleic acid variations are “silent variations,” which are one species of conservatively modified variations. Every nucleic acid sequence herein which encodes a polypeptide also describes silent variations of the nucleic acid. One of skill will recognize that in certain contexts each codon in a nucleic acid (except AUG, which is ordinarily the only codon for methionine, and TGG, which is ordinarily the only codon for tryptophan) can be modified to yield a functionally identical molecule. Accordingly, silent variations of a nucleic acid which encodes a polypeptide is implicit in a described sequence with respect to the expression product, but not with respect to actual probe sequences.

As to amino acid sequences, one of skill will recognize that individual substitutions, deletions or additions to a nucleic acid, peptide, polypeptide, or protein sequence which alters, adds or deletes a single amino acid or a small percentage of amino acids in the encoded sequence is a “conservatively modified variant” where the alteration results in the substitution of an amino acid with a chemically similar amino acid. Conservative substitution tables providing functionally similar amino acids are well known in the art. Such conservatively modified variants are in addition to and do not exclude polymorphic variants, interspecies homologs, and alleles of the invention. The following amino acids are typically conservative substitutions for one another: 1) Alanine (A), Glycine (G); 2) Aspartic acid (D), Glutamic acid (E); 3) Asparagine (N), Glutamine (Q); 4) Arginine (R), Lysine (K); 5) Isoleucine (I), Leucine (L), Methionine (M), Valine (V); 6) Phenylalanine (F), Tyrosine (Y), Tryptophan (W); 7) Serine(S), Threonine (T); and 8) Cysteine (C), Methionine (M) (see, e.g., Creighton, Proteins (1984)).

The terms “identical” or “percent identity,” in the context of two or more nucleic acids, or two or more polypeptides, refer to two or more sequences or subsequences that are the same or have a specified percentage of nucleotides, or amino acids, that are the same (i.e., about 60% identity, preferably 65%, 70%, 75%, 80%, 85%, 90%, 91%, 92%, 93%, 94%, 95%, 96%, 97%, 98%, 99%, or higher identity over a specified region, when compared and aligned for maximum correspondence over a comparison window or designated region) as measured using a BLAST or BLAST 2.0 sequence comparison algorithms with default parameters, or by manual alignment and visual inspection. See e.g., the NCBI web site at ncbi.nlm.nih.gov/BLAST. Such sequences are then said to be “substantially identical.” This definition also refers to, or may be applied to, the compliment of a nucleotide test sequence. The definition also includes sequences that have deletions and/or additions, as well as those that have substitutions. As described below, the algorithms can account for gaps and the like. Typically, identity exists over a region comprising an antibody epitope, or a sequence that is at least about 25 amino acids or nucleotides in length, or over a region that is 50-100 amino acids or nucleotides in length, or over the entire length of the reference sequence.

The term “recombinant” when used with reference, e.g., to a cell, or nucleic acid, protein, or vector, indicates that the cell, nucleic acid, protein or vector, has been modified by the introduction of a heterologous nucleic acid or protein or the alteration of a native nucleic acid or protein, or that the cell is derived from a cell so modified. Thus, for example, recombinant cells express genes that are not found within the native (non-recombinant) form of the cell or express native genes that are otherwise abnormally expressed, under expressed or not expressed at all.

The term “heterologous” when used with reference to portions of a nucleic acid indicates that the nucleic acid comprises two or more subsequences that are not found in the same relationship to each other in nature. For instance, the nucleic acid is typically recombinantly produced, having two or more sequences from unrelated genes arranged to make a new functional nucleic acid, e.g., a promoter from one source and a coding region from another source. Similarly, a heterologous protein indicates that the protein comprises two or more subsequences that are not found in the same relationship to each other in nature (e.g., a fusion protein).

The term “isolated,” when applied to a nucleic acid or protein, denotes that the nucleic acid or protein is essentially free of other cellular components with which it is associated in the natural state. It is preferably in a homogeneous state. It can be in either a dry or aqueous solution. Purity and homogeneity are typically determined using analytical chemistry techniques such as polyacrylamide gel electrophoresis or high performance liquid chromatography. A protein that is the predominant species present in a preparation is substantially purified. In particular, an isolated gene is separated from open reading frames that flank the gene and encode a protein other than the gene of interest. The term “purified” denotes that a nucleic acid or protein gives rise to essentially one band in an electrophoretic gel. Particularly, it means that the nucleic acid or protein is at least 85% pure, more preferably at least 95% pure, and most preferably at least 99% pure.

The term “antibody” refers to a polypeptide comprising a framework region encoded by an immunoglobulin gene, or fragments thereof, that specifically bind and recognize an antigen, e.g., human αvβ8, a particular cell surface marker, or any desired target. Typically, the “variable region” contains the antigen-binding region of the antibody (or its functional equivalent) and is most critical in specificity and affinity of binding. See Paul, Fundamental Immunology (2003).

An exemplary immunoglobulin (antibody) structural unit comprises a tetramer. Each tetramer is composed of two identical pairs of polypeptide chains, each pair having one “light” (about 25 kD) and one “heavy” chain (about 50-70 kD). The N-terminus of each chain defines a variable region of about 100 to 110 or more amino acids primarily responsible for antigen recognition. The terms variable light chain (VL) and variable heavy chain (VH) refer to these light and heavy chains respectively.

An “isotype” is a class of antibodies defined by the heavy chain constant region. Antibodies described herein can be of any isotype of isotype class. Immunoglobulin genes include the kappa, lambda, alpha, gamma, delta, epsilon, and mu constant region genes. Light chains are classified as either kappa or lambda. Heavy chains are classified as gamma, mu, alpha, delta, or epsilon, which in turn define the isotype classes, IgG, IgM, IgA, IgD and IgE, respectively. In some embodiments, the IgG is an IgG1, IgG2, IgG3 or IgG4.

Antibodies can exist as intact immunoglobulins or as any of a number of well-characterized fragments that include specific antigen-binding activity. Such fragments can be produced by digestion with various peptidases. Pepsin digests an antibody below the disulfide linkages in the hinge region to produce F(ab)′₂. a dimer of Fab which itself is a light chain joined to V_{H}-C_{H}1 by a disulfide bond. The F(ab)′₂ may be reduced under mild conditions to break the disulfide linkage in the hinge region, thereby converting the F(ab)′₂ dimer into an Fab′ monomer. The Fab′ monomer is essentially Fab with part of the hinge region (see Fundamental Immunology (Paul ed., 3d ed. 1993). While various antibody fragments are defined in terms of the digestion of an intact antibody, one of skill will appreciate that such fragments may be synthesized de novo either chemically or by using recombinant DNA methodology. Thus, the term antibody, as used herein, also includes antibody fragments either produced by the modification of whole antibodies, or those synthesized de novo using recombinant DNA methodologies (e.g., single chain Fv) or those identified using phage display libraries (see, e.g., McCafferty et al., Nature 348:552-554 (1990)).

For preparation of monoclonal or polyclonal antibodies, any technique known in the art can be used (see, e.g., Kohler & Milstein, Nature 256:495-497 (1975); Kozbor et al., Immunology Today 4:72 (1983); Cole et al., Monoclonal Antibodies and Cancer Therapy, pp. 77-96. Alan R. Liss, Inc. 1985). Techniques for the production of single chain antibodies (U.S. Pat. No. 4,946,778) can be adapted to produce antibodies to polypeptides of this invention. Also, transgenic mice, or other organisms such as other mammals, may be used to express humanized antibodies. Alternatively, phage display technology can be used to identify antibodies and heteromeric Fab fragments that specifically bind to selected antigens (see, e.g., McCafferty et al., supra; Marks et al., Biotechnology, 10:779-783, (1992)).

Methods for humanizing or primatizing non-human antibodies are well known in the art. Generally, a humanized antibody has one or more amino acid residues introduced into it from a source which is non-human. These non-human amino acid residues are often referred to as import residues, which are typically taken from an import variable domain. Antibody humanization can be performed using a variety of methods known in the art including but not limited to, methods of Winter and co-workers (see, e.g., Jones et al., Nature 321:522-525 (1986); Riechmann et al., Nature 332:323-327 (1988); Verhoeyen et al., Science 239:1534-1536 (1988) and Presta, Curr. Op. Struct. Biol. 2:593-596 (1992)), which substitute specific rodent CDRs or CDR sequences for the corresponding sequences of a human antibody. Accordingly, such humanized antibodies are chimeric antibodies (U.S. Pat. No. 4,816,567), wherein substantially less than an intact human variable domain has been substituted by the corresponding sequence from a non-human species. In practice, humanized antibodies are typically human antibodies in which some complementary determining region (“CDR”) residues and possibly some framework (“FR”) residues are substituted by residues from analogous sites in rodent antibodies so as to introduce favorable properties including but not limited to affinity, stability, expression, and solubility.

Antibodies or antigen-binding molecules of the invention further includes one or more immunoglobulin chains that are chemically conjugated to, or expressed as, fusion proteins with other proteins. It also includes bispecific antibody. A bispecific or bifunctional antibody is an artificial hybrid antibody having two different heavy/light chain pairs and two different binding sites. Other antigen-binding fragments or antibody portions of the invention include bivalent scFv (diabody), bispecific scFv antibodies where the antibody molecule recognizes two different epitopes, single binding domains (dAbs), and minibodies.

The various antibodies or antigen-binding fragments described herein can be produced by enzymatic or chemical modification of the intact antibodies, or synthesized de novo using recombinant DNA methodologies (e.g., single chain Fv), or identified using yeast or phage display libraries (see, e.g., McCafferty et al., Nature 348:552-554, 1990; Boder, et al (2000) Proc. Natl. Acad. Sci. U.S.A 97:10701). For example, minibodies can be generated using methods described in the art, e.g., Vaughan and Sollazzo, Comb Chem High Throughput Screen. 4:417-30 2001. Bispecific antibodies can be produced by a variety of methods including fusion of hybridomas or linking of Fab′ fragments. See, e.g., Songsivilai & Lachmann, Clin. Exp. Immunol. 79:315-321 (1990); Kostelny et al., J. Immunol. 148, 1547-1553 (1992). Single chain antibodies can be identified using phage display libraries, yeast display, or ribosome display libraries, gene shuffled libraries. Such libraries can be constructed from synthetic, semi-synthetic or native and immunocompetent sources.

A “monoclonal antibody” refers to a clonal preparation of antibodies with a single binding specificity and affinity for a given epitope on an antigen. A “polyclonal antibody” refers to a preparation of antibodies that are raised against a single antigen, but with different binding specificities and affinities.

As used herein, “V-region” refers to an antibody variable region domain comprising the segments of Framework 1, CDR1, Framework 2, CDR2, Framework 3, CDR3, and Framework 4. These segments are included in the V-segment as a consequence of rearrangement of the heavy chain and light chain V-region genes during B-cell differentiation.

As used herein, “complementarity-determining region (CDR)” refers to the three hypervariable regions in each chain that interrupt the four “framework” regions established by the light and heavy chain variable regions. The CDRs are primarily responsible for binding to an epitope of an antigen. The CDRs of each chain are typically referred to as CDR1, CDR2, and CDR3, numbered sequentially starting from the N-terminus, and are also typically identified by the chain in which the particular CDR is located. Thus, a V_{H} CDR3 is located in the variable domain of the heavy chain of the antibody in which it is found, whereas a V_{L} CDR1 is the CDR1 from the variable domain of the light chain of the antibody in which it is found.

The sequences of the framework regions of different light or heavy chains are relatively conserved within a species. The framework region of an antibody, that is the combined framework regions of the constituent light and heavy chains, serves to position and align the CDRs in three dimensional space.

The amino acid sequences of the CDRs and framework regions can be determined using various well known definitions in the art, e.g., Kabat, Chothia, international ImMunoGeneTics database (IMGT), and AbM (see, e.g., Johnson and Wu, Nucleic Acids Res. 2000 Jan. 1; 28 (1): 214-218 and Johnson et al., Nucleic Acids Res., 29:205-206 (2001); Chothia & Lesk, (1987) J. Mol. Biol. 196, 901-917; Chothia et al. (1989) Nature 342, 877-883; Chothia et al. (1992) J. Mol. Biol. 227, 799-817; Al-Lazikani et al., J. Mol. Biol 1997, 273 (4)). Unless otherwise indicated, CDRs are determined according to Kabat. Definitions of antigen combining sites are also described in the following: Ruiz et al. Nucleic Acids Res., 28, 219-221 (2000); and Lefranc Nucleic Acids Res. January 1; 29 (1): 207-9 (2001); MacCallum et al., J. Mol. Biol., 262:732-745 (1996); and Martin et al, Proc. Natl Acad. Sci. USA, 86, 9268-9272 (1989); Martin, et al, Methods Enzymol., 203:121-153, (1991); Pedersen et al, Immunomethods, 1, 126, (1992); and Rees et al, In Sternberg M.J.E. (ed.), Protein Structure Prediction. Oxford University Press, Oxford, 141-172 1996).

A “chimeric antibody” is an antibody molecule in which (a) the constant region, or a portion thereof, is altered, replaced or exchanged so that the antigen binding site (variable region, CDR, or portion thereof) is linked to a constant region of a different or altered class, effector function and/or species, or an entirely different molecule which confers new properties to the chimeric antibody (e.g., an enzyme, toxin, hormone, growth factor, drug, etc.); or (b) the variable region, or a portion thereof, is altered, replaced or exchanged with a variable region having a different or altered antigen specificity (e.g., CDR and framework regions from different species).

A “humanized” antibody is an antibody that retains the reactivity of a non-human antibody while being less immunogenic in humans. This can be achieved, for instance, by retaining the non-human CDR regions and replacing the remaining parts of the antibody with their human counterparts. In one embodiment, some, most or all of the amino acids outside the CDR domains are replaced with amino acids corresponding to the human immunoglobulin germline, while amino acids within one or more CDR regions are unchanged or altered. Some amino acids in framework regions may form direct or indirect interactions with CDR residues and thus influence the shape of the CDR and its ability to interact with its antigen. Such residues are herein referred to as “Vernier zone” residues as described by Foote and Winter (1992, J. Mol. Biol. 224:487-499). The humanization of Vernier residues frequently impedes the success of commonly used humanization strategies including but not limited to CDR grafting, specificity determining region grafting (Kim and Hong (2012) Methods Mol Biol, 907:237-45), resurfacing or veneering (Desmet, et al. (2010) Antibody Engineering. Springer Protocols Handbooks). If humanization of Vernier or non-Vernier framework residues result in loss of antibody affinity, rational or empiric design strategies can be employed. Rational design methods are facilitated if a structure of the antibody-antigen complex is available. As such, individual amino acids can be predicted, based on their biophysical properties, to improve binding to a specific antibody-antigen protein interface. By generating a small set of rationally designed variants, based on the antibody structure and sequence information, binding affinity or any other characteristic of interest, can be readily assessed. Empiric methods (i.e. combinatorial libraries) can also allow screening of many different amino acids in Vernier zones or other regions of interest to overcome humanization obstacles. Combining rational design approaches with empirical methods can achieve so called “directed evolution” to produce antibodies with desired sequences and properties. These properties may include improved humanization and antibody biophysical properties while preserving antibody binding and specificity. See, e.g., Morrison et al., Proc. Natl. Acad. Sci. USA, 81:6851-6855 (1984); Morrison and Oi, Adv. Immunol., 44:65-92 (1988); Verhoeyen et al., Science, 239:1534-1536 (1988); Padlan, Molec. Immun., 28:489-498 (1991); Padlan, Molec. Immun., 31 (3): 169-217 (1994).

The regions of the light chains not constituting the variable domains contain a constant domain (CH1), that is shared between different immunoglobulin subtypes. The light chain CH1 interacts with the heavy chain CH1 domain. The remaining heavy chain domains include a hinge region and the CH2 and CH3 domains, the latter constituting one-half of the homodimeric Fc region. Generally, the variable domains describe herein may be linked to the hinge and Fc domains of any human or non-human Fc. The hinge and Fc domains may contain one or more modifications including but not limited to those that alter thermodynamic stability, pharmacokinetic properties, complement fixation, Fc-receptor binding and/or antigen-dependent cytotoxicity (ADCC). Complement fixation and ADCC are known broadly as antibody “effector functions”.

The antibody binds to an “epitope” on the antigen. The epitope is the specific antibody binding interaction site on the antigen, and can include a few amino acids or portions of a few amino acids, e.g., 5 or 6, or more, e.g., 20 or more amino acids, or portions of those amino acids. In some cases, the epitope includes non-protein components, e.g., from a carbohydrate, nucleic acid, or lipid. In some cases, the epitope is a three-dimensional moiety. Thus, for example, where the target is a protein, the epitope can be comprised of consecutive amino acids, or amino acids from different parts of the protein that are brought into proximity by protein folding (e.g., a discontinuous epitope). The same is true for other types of target molecules that form three-dimensional structures.

The term “specifically bind” refers to a molecule (e.g., antibody or antibody fragment) that binds to a target with at least 2-fold greater affinity than non-target compounds, e.g., at least 4-fold, 5-fold, 6-fold, 7-fold, 8-fold, 9-fold, 10-fold, 20-fold, 25-fold, 50-fold, or 100-fold greater affinity. For example, an antibody that specifically binds β8 will typically bind to β8 with at least a 2-fold greater affinity than a non-β8 target (e.g., a different integrin subunit, e.g., β6).

The term “binds” with respect to a cell type (e.g., an antibody that binds fibrotic cells, hepatocytes, chondrocytes, etc.), typically indicates that an agent binds a majority of the cells in a pure population of those cells. For example, an antibody that binds a given cell type typically binds to at least ⅔ of the cells in a population of the indicated cells (e.g., 75, 80, 85, 90, 91, 92, 93, 94, 95, 96, 97, 98, 99, or 100%). One of skill will recognize that some variability will arise depending on the method and/or threshold of determining binding.

As used herein, a first antibody, or an antigen-binding portion thereof, “competes” for binding to a target with a second antibody, or an antigen-binding portion thereof, when binding of the second antibody with the target is detectably decreased in the presence of the first antibody compared to the binding of the second antibody in the absence of the first antibody. The alternative, where the binding of the first antibody to the target is also detectably decreased in the presence of the second antibody, can, but need not be the case. That is, a second antibody can inhibit the binding of a first antibody to the target without that first antibody inhibiting the binding of the second antibody to the target. However, where each antibody detectably inhibits the binding of the other antibody to its cognate epitope or ligand, whether to the same, greater, or lesser extent, the antibodies are said to “cross-compete” with each other for binding of their respective epitope(s). Both competing and cross-competing antibodies are encompassed by the present invention. The term “competitor” antibody can be applied to the first or second antibody as can be determined by one of skill in the art. In some cases, the presence of the competitor antibody (e.g., the first antibody) reduces binding of the second antibody to the target by at least 10%, e.g., 20%, 30%, 40%, 50%, 60%, 70%, 80%, or more, e.g., so that binding of the second antibody to target is undetectable in the presence of the first (competitor) antibody.

A “label” or a “detectable moiety” is a composition detectable by spectroscopic, photochemical, biochemical, immunochemical, chemical, or other physical means. For example, useful labels include 32P, fluorescent dyes, electron-dense reagents, enzymes (e.g., as commonly used in an ELISA), biotin, digoxigenin, or haptens and proteins or other entities which can be made detectable, e.g., by incorporating a radiolabel into a peptide or antibody specifically reactive with a target peptide. Any method known in the art for conjugating an antibody to the label may be employed, e.g., using methods described in Hermanson, Bioconjugate Techniques 1996, Academic Press, Inc., San Diego.

A “labeled” molecule (e.g., nucleic acid, protein, or antibody) is one that is bound, either covalently, through a linker or a chemical bond, or noncovalently, through ionic, van der Waals, electrostatic, or hydrogen bonds to a label such that the presence of the molecule may be detected by detecting the presence of the label bound to the molecule.

The term “diagnosis” refers to a relative probability that a disorder such as cancer or an inflammatory condition is present in the subject. Similarly, the term “prognosis” refers to a relative probability that a certain future outcome may occur in the subject. For example, prognosis can refer to the likelihood that an individual will develop a TGFβ or αvβ8 associated disorder, have recurrence, or the likely severity of the disease (e.g., severity of symptoms, rate of functional decline, survival, etc.). The terms are not intended to be absolute, as will be appreciated by any one of skill in the field of medical diagnostics.

“Biopsy” or “biological sample from a patient” as used herein refers to a sample obtained from a patient having, or suspected of having, a TGFβ or αvβ8 associated disorder. In some embodiments, the sample may be a tissue biopsy, such as needle biopsy, fine needle biopsy, surgical biopsy, etc. The sample can also be a blood sample or blood fraction, e.g., white blood cell fraction, serum, or plasma. The sample can comprise a tissue sample harboring a lesion or suspected lesion, although the biological sample may be also be derived from another site, e.g., a site of suspected metastasis, a lymph node, or from the blood. In some cases, the biological sample may also be from a region adjacent to the lesion or suspected lesion.

A “biological sample” can be obtained from a patient, e.g., a biopsy, from an animal, such as an animal model, or from cultured cells, e.g., a cell line or cells removed from a patient and grown in culture for observation. Biological samples include tissues and bodily fluids, e.g., blood, blood fractions, lymph, saliva, urine, feces, etc.

The terms “therapy,” “treatment,” and “amelioration” refer to any reduction in the severity of symptoms. In the case of treating an inflammatory condition, the treatment can refer to reducing, e.g., blood levels of inflammatory cytokines, blood levels of active mature TGFβ, pain, swelling, recruitment of immune cells, etc. In the case of treating cancer, treatment can refer to reducing, e.g., tumor size, number of cancer cells, growth rate, metastatic activity, cell death of non-cancer cells, etc. As used herein, the terms “treat” and “prevent” are not intended to be absolute terms. Treatment and prevention can refer to any delay in onset, amelioration of symptoms, improvement in patient survival, increase in survival time or rate, etc. Treatment and prevention can be complete (no detectable symptoms remaining) or partial, such that symptoms are less frequent of severe than in a patient without the treatment described herein. The effect of treatment can be compared to an individual or pool of individuals not receiving the treatment, or to the same patient prior to treatment or at a different time during treatment. In some aspects, the severity of disease is reduced by at least 10%, as compared, e.g., to the individual before administration or to a control individual not undergoing treatment. In some aspects the severity of disease is reduced by at least 25%, 50%, 75%, 80%, or 90%, or in some cases, no longer detectable using standard diagnostic techniques.

The terms “effective amount,” “effective dose,” “therapeutically effective amount,” etc. refer to that amount of the therapeutic agent sufficient to ameliorate a disorder, as described above. For example, for the given parameter, a therapeutically effective amount will show an increase or decrease of therapeutic effect at least 5%, 10%, 15%, 20%, 25%, 40%, 50%, 60%, 75%, 80%, 90%, or at least 100%. Therapeutic efficacy can also be expressed as “-fold” increase or decrease. For example, a therapeutically effective amount can have at least a 1.2-fold, 1.5-fold, 2-fold, 5-fold, or more effect over a control.

As used herein, the term “pharmaceutically acceptable” is used synonymously with physiologically acceptable and pharmacologically acceptable. A pharmaceutical composition will generally comprise agents for buffering and preservation in storage, and can include buffers and carriers for appropriate delivery, depending on the route of administration.

The terms “dose” and “dosage” are used interchangeably herein. A dose refers to the amount of active ingredient given to an individual at each administration. For the present invention, the dose can refer to the concentration of the antibody or associated components, e.g., the amount of therapeutic agent or dosage of radiolabel. The dose will vary depending on a number of factors, including frequency of administration; size and tolerance of the individual; severity of the condition; risk of side effects; the route of administration; and the imaging modality of the detectable moiety (if present). One of skill in the art will recognize that the dose can be modified depending on the above factors or based on therapeutic progress. The term “dosage form” refers to the particular format of the pharmaceutical, and depends on the route of administration. For example, a dosage form can be in a liquid, e.g., a saline solution for injection.

“Subject,” “patient,” “individual” and like terms are used interchangeably and refer to, except where indicated, mammals such as humans and non-human primates, as well as rabbits, rats, mice, goats, pigs, and other mammalian species. The term does not necessarily indicate that the subject has been diagnosed with a particular disease, but typically refers to an individual under medical supervision. A patient can be an individual that is seeking treatment, monitoring, adjustment or modification of an existing therapeutic regimen, etc.

“Cancer”, “tumor,” “transformed” and like terms include precancerous, neoplastic, transformed, and cancerous cells, and can refer to a solid tumor, or a non-solid cancer (see, e.g., Edge et al. AJCC Cancer Staging Manual (7^{th} ed. 2009); Cibas and Ducatman Cytology: Diagnostic principles and clinical correlates (3^{rd} ed. 2009)). Cancer includes both benign and malignant neoplasms (abnormal growth). “Transformation” refers to spontaneous or induced phenotypic changes, e.g., immortalization of cells, morphological changes, aberrant cell growth, reduced contact inhibition and anchorage, and/or malignancy (see, Freshney, Culture of Animal Cells a Manual of Basic Technique (3^{rd} ed. 1994)). Although transformation can arise from infection with a transforming virus and incorporation of new genomic DNA, or uptake of exogenous DNA, it can also arise spontaneously or following exposure to a carcinogen.

The term “cancer” can refer to carcinomas, sarcomas, adenocarcinomas, lymphomas, leukemias, solid and lymphoid cancers, etc. Examples of different types of cancer include, but are not limited to, lung cancer (e.g., non-small cell lung cancer or NSCLC), ovarian cancer, prostate cancer, colorectal cancer, liver cancer (i.e., hepatocarcinoma), renal cancer (i.e., renal cell carcinoma), bladder cancer, breast cancer, thyroid cancer, pleural cancer, pancreatic cancer, uterine cancer, cervical cancer, testicular cancer, anal cancer, pancreatic cancer, bile duct cancer, gastrointestinal carcinoid tumors, esophageal cancer, gall bladder cancer, appendix cancer, small intestine cancer, stomach (gastric) cancer, cancer of the central nervous system, skin cancer, choriocarcinoma; head and neck cancer, blood cancer, osteogenic sarcoma, fibrosarcoma, neuroblastoma, glioma, melanoma, B-cell lymphoma, non-Hodgkin's lymphoma, Burkitt's lymphoma, Small Cell lymphoma, Large Cell lymphoma, monocytic leukemia, myelogenous leukemia, acute lymphocytic leukemia, acute myelocytic leukemia (AML), chronic myeloid leukemia (CML), and multiple myeloma. In some embodiments, the antibody compositions and methods described herein can be used for treating cancer.

The term “co-administer” refers to the simultaneous presence of two active agents in the blood of an individual. Active agents that are co-administered can be concurrently or sequentially delivered.

The terms “chimeric antigen receptor” and “CAR”, used interchangeably herein, refer to artificial multi-module molecules capable of triggering or inhibiting the activation of an immune cell which generally but not exclusively comprise an extracellular domain (e.g., a ligand/antigen binding domain), a transmembrane domain and one or more intracellular signaling domains. The term CAR is not limited specifically to CAR molecules but also includes CAR variants. CAR variants include split CARs wherein the extracellular portion (e.g., the ligand binding portion) and the intracellular portion (e.g., the intracellular signaling portion) of a CAR are present on two separate molecules. CAR variants also include ON-switch CARs which are conditionally activatable CARs, e.g., comprising a split CAR wherein conditional hetero-dimerization of the two portions of the split CAR is pharmacologically controlled. CAR variants also include bispecific CARs, which include a secondary CAR binding domain that can either amplify or inhibit the activity of a primary CAR. CAR variants also include inhibitory chimeric antigen receptors (iCARs) which may, e.g., be used as a component of a bispecific CAR system, where binding of a secondary CAR binding domain results in inhibition of primary CAR activation. CAR molecules and derivatives thereof (i.e., CAR variants) are described, e.g., in PCT Application No. US2014/016527; Fedorov et al. Sci Transl Med (2013); 5 (215): 215ra172; Glienke et al. Front Pharmacol (2015) 6:21; Kakarla & Gottschalk 52 Cancer J (2014) 20 (2): 151-5; Riddell et al. Cancer J (2014) 20 (2): 141-4; Pegram et al. Cancer J (2014) 20 (2): 127-33; Cheadle et al. Immunol Rev (2014) 257 (1): 91-106; Barrett et al. Annu Rev Med (2014) 65:333-47; Sadelain et al. Cancer Discov (2013) 3 (4): 388-98; Cartellieri et al., J Biomed Biotechnol (2010) 956304; the disclosures of which are incorporated herein by reference in their entirety.

As used herein, the term “immune cells” generally includes white blood cells (leukocytes) which are derived from hematopoietic stem cells (HSC) produced in the bone marrow “Immune cells” includes, e.g., lymphocytes (T cells, B cells, natural killer (NK) cells) and myeloid-derived cells (neutrophil, eosinophil, basophil, monocyte, macrophage, dendritic cells).

“T cell” includes all types of immune cells expressing CD3 including T-helper cells (CD4+ cells), cytotoxic T-cells (CD8+ cells), T-regulatory cells (Treg) and gamma-delta T cells.

A “cytotoxic cell” includes CD8+ T cells, natural-killer (NK) cells, and neutrophils, which cells are capable of mediating cytotoxicity responses.

### DETAILED DESCRIPTION OF THE INVENTION

The inventors have created new more fully humanized antibodies with the same or higher affinity for αvβ8 compared to earlier-described less humanized antibodies while minimizing introduction of sequence liabilities that can hinder the antibody development process and that in some cases improve expression. For example, compared to the HuC6D4F12 (also known herein as F12 or HuF12) antibody as described in PCT Publication WO 2021/146614, the present disclosure provides new sequences that include, for example, changes in various complementarity-determining regions (CDRs) and/or framework regions (FR) that maintain or improve affinity, reduce potential immunogenicity, or both.

For example, relative to the HuC6D4F12 antibody noted above, the inventors have found the following advantageous changes:

VH CDR1:
1.
KYSMH (HuC6D4F12) to RQSMH (improves affinity during further humanization of
HuC6D4F12);
or
2.
KYSMH (HuC6D4F12) to EQSMH (improves the charge distribution during antibody
humanization without significant loss in other antibody properties).
VK CDR1:
1.
(SEQ ID NO: 8)
KSSQSLLHSRSRKNYLA to
(SEQ ID NO: 4)
RSSQSLLHSRSRKNYLA
(improves affinity during further humanization of HuC6D4F12).
VH FR3
1.
L (in F12 FR3: RFTVTLDTSTSTAYLEIRSLRSDDTAVYFCAI, SEQ ID NO: 56) to
(SEQ ID NO: 14)
Q or E (RVTMTQDTSTDTAYMELSSLRSEDTAVYFCAI (improves
affinity during further humanization of HuC6D4F12).

The above changes are particularly useful to compensate for the loss in affinity in the context of humanized frameworks, in which affinity can otherwise be reduced.

Accordingly, in some embodiments, the antibodies described herein specifically bind to αvβ8. For example, the antibodies do not substantially bind to integrins αvβ1, αvβ3, αvβ5 or αvβ6. Variable regions of some of the antibodies described herein are displayed in Tables VIII and IX. Exemplary antibodies can for example, comprise a heavy chain variable region comprising heavy chain CDRs 1, 2, and 3, a light chain variable region comprising a light chain CDRs 1, 2, and 3, or combinations thereof, as follows:

A HCDR1 comprising
(SEQ ID NO: 1)
RQSMH
or
(SEQ ID NO: 7)
EQSMH;
a HCDR2 comprising
(SEQ ID NO: 2)
RINTETGEPTYAQKFQG;
and
a HCDR3 comprising
(SEQ ID NO: 3)
FYYGRD(S/T);
and/or
A LCDR1 comprising
(SEQ ID NO: 4)
RSSQSLLHSRSRKNYLA;
a LCDR2 comprising
(SEQ ID NO: 5)
WASTRET;
and
a LCDR3 comprising
(SEQ ID NO: 6)
KQSYNLLS.

For example, the HCDR3 in the combinations above can comprise FYYGRDS (SEQ ID NO:57) or FYYGRDT (SEQ ID NO:58). Thus, in some embodiments, the heavy chain variable region comprises:
SEQ ID NO:1, SEQ ID NO:2 and SEQ ID NO:57; or
SEQ ID NO:1, SEQ ID NO:2 and SEQ ID NO:58; or
SEQ ID NO:7, SEQ ID NO:2 and SEQ ID NO:57; or
SEQ ID NO:7, SEQ ID NO:2 and SEQ ID NO:58 and
the light chain variable region comprises SEQ ID NO: 4, SEQ ID NO:5 and SEQ ID NO:6.

In some embodiments, the antibodies comprise the heavy chain CDR1, CDR2, and CDR3 sequences described above but contain 1, 2, or 3 amino acid substitutions in one, two or more CDR sequences compared to those listed above, but includes the underlined amino acid in HCDR1 ((E/R)QSMH). In some embodiments, the antibodies comprise the light chain CDR1, CDR2, and CDR3 sequences described above but contain 1, 2, or 3 amino acid substitutions in one, two or more CDR sequences compared to those listed above, but includes the optionally conservative substitution underlined amino acid in LCDR1 (RSSQSLLRRGDLATIHGNALA (SEQ ID NO:9)).

The CDRs described above can be inserted into any framework sequences as desired, include, for example, those described herein. For example, a heavy chain variable region comprising heavy chain FRI can comprise SEQ ID NO:12, heavy chain FR2 can comprise SEQ ID NO: 13, heavy chain FR3 can comprise SEQ ID NO:14 or SEQ ID NO:21 and heavy chain FR4 can comprise SEQ ID NO:15, optionally combined with a light chain variable region comprising light chain FRI comprising SEQ ID NO:16, light chain FR2 comprising SEQ ID NO: 17, light chain FR3 comprising SEQ ID NO: 18 and light chain FR4 comprising SEQ ID NO: 19 or SEQ ID NO:20.

Exemplary heavy and light chain variable region pairings with RGD sequences as described above include but are not limited to wherein:
the heavy chain variable region comprises: SEQ ID NO:34 and the light chain variable region comprises: SEQ ID NO:35; or
the heavy chain variable region comprises: SEQ ID NO:34 and the light chain variable region comprises: SEQ ID NO:36; or
the heavy chain variable region comprises: SEQ ID NO:37 and the light chain variable region comprises: SEQ ID NO:35; or
the heavy chain variable region comprises: SEQ ID NO:37 and the light chain variable region comprises: SEQ ID NO:36; or
the heavy chain variable region comprises: SEQ ID NO:38 and the light chain variable region comprises: SEQ ID NO:35; or
the heavy chain variable region comprises: SEQ ID NO:38 and the light chain variable region comprises: SEQ ID NO:36; or
the heavy chain variable region comprises: SEQ ID NO:39 and the light chain variable region comprises: SEQ ID NO:35; or
the heavy chain variable region comprises: SEQ ID NO:39 and the light chain variable region comprises: SEQ ID NO:36.

A number of humanized variants in the framework regions are also described herein that can be used independently or together in a framework. For example, the underlined amino acids represent changes from the indicated F12 framework regions:

VH FR1:
(SEQ ID NO: 12)
QVQLVQSGAEVKKPGASVKVSCKVSGYTLT
VHFR2:
(SEQ ID NO: 13)
WVRQAPGKGLEWMG
VH FR3:
(SEQ ID NO: 14)
RVTMTQDTSTDTAYMELSSLRSEDTAVYFCAI
VH FR4
(SEQ ID NO: 15)
WGQGTTVTVSS
VK FR1:
(SEQ ID NO: 16)
EIVMTQSPATLSVSPGERVTLSC
VK FR3:
(SEQ ID NO: 18)
GVPARFSGSGSGTEFTLTISSLQSEDFAVYYC
VK FR4:
(SEQ ID NO: 19)
FGQGTKVEIKR

In some embodiments, any antibody described herein can comprise a light chain CDR1 comprising a RGD or RGDL sequence from TGF-β3, for example GRGDLGRLKK. Inclusion of such a sequence in the light chain CDR1 allows the antibody to additionally bind to αvβ6, as well as to αvβ8. Exemplary light chain CDR1 sequences can comprise, for example, SEQ ID NO: 8, SEQ ID NO:9, SEQ ID NO: 10 or SEQ ID NO:11.

Exemplary antibodies can for example, comprise a heavy chain variable region comprising heavy chain CDRs 1, 2, and 3, a light chain variable region comprising a light chain CDRs 1, 2, and 3, or combinations thereof, as follows:
A HCDR1 comprising SEQ ID NO: 1 or SEQ ID NO:7; a HCDR 2 comprising SEQ ID NO:2; and a HCDR 3 comprising SEQ ID NO:3; and/or

A LCDR1 comprising
(SEQ ID NO: 8)
KSSQSLLGRGDLGRLKKNALA
or
(SEQ ID NO: 9)
RSSQSLLRRGDLATIHGNALA
or
(SEQ ID NO: 10)
KSSQSLLRRGDLATIHGNALA
or
(SEQ ID NO: 11)
RSSQSLLRRGDLATIHGNALA;
a LCDR2 comprising SEQ ID NO: 5;
and
a LCDR3 comprising SEQ ID NO: 6.

The CDRs described above can be inserted into any framework sequences as desired, include, for example, those described herein. Exemplary heavy and light chain variable region pairings with RGD sequences as described above include but are not limited to wherein: the heavy chain variable region comprises: SEQ ID NO:34 and the light chain variable region comprises: SEQ ID NO:40; or
the heavy chain variable region comprises: SEQ ID NO:34 and the light chain variable region comprises: SEQ ID NO:41; or
the heavy chain variable region comprises: SEQ ID NO:37 and the light chain variable region comprises: SEQ ID NO:40; or
the heavy chain variable region comprises: SEQ ID NO:37 and the light chain variable region comprises: SEQ ID NO:41; or
the heavy chain variable region comprises: SEQ ID NO:34 and the light chain variable region comprises: SEQ ID NO:42; or
the heavy chain variable region comprises: SEQ ID NO:34 and the light chain variable region comprises: SEQ ID NO:43; or
the heavy chain variable region comprises: SEQ ID NO:37 and the light chain variable region comprises: SEQ ID NO:42; or
the heavy chain variable region comprises: SEQ ID NO:37 and the light chain variable region comprises: SEQ ID NO:43; or
the heavy chain variable region comprises: SEQ ID NO:38 and the light chain variable region comprises: SEQ ID NO:40; or
the heavy chain variable region comprises: SEQ ID NO:38 and the light chain variable region comprises: SEQ ID NO:41; or
the heavy chain variable region comprises: SEQ ID NO:38 and the light chain variable region comprises: SEQ ID NO:42; or
the heavy chain variable region comprises: SEQ ID NO:38 and the light chain variable region comprises: SEQ ID NO:43; or
the heavy chain variable region comprises: SEQ ID NO:39 and the light chain variable region comprises: SEQ ID NO:40; or
the heavy chain variable region comprises: SEQ ID NO:39 and the light chain variable region comprises: SEQ ID NO:41; or
the heavy chain variable region comprises: SEQ ID NO:39 and the light chain variable region comprises: SEQ ID NO:42; or
the heavy chain variable region comprises: SEQ ID NO:39 and the light chain variable region comprises: SEQ ID NO:43; or
the heavy chain variable region comprises: SEQ ID NO:34 and the light chain variable region comprises: SEQ ID NO:44; or
the heavy chain variable region comprises: SEQ ID NO:34 and the light chain variable region comprises: SEQ ID NO:45; or
the heavy chain variable region comprises: SEQ ID NO:37 and the light chain variable region comprises: SEQ ID NO:44; or
the heavy chain variable region comprises: SEQ ID NO:37 and the light chain variable region comprises: SEQ ID NO:45; or
the heavy chain variable region comprises: SEQ ID NO:34 and the light chain variable region comprises: SEQ ID NO:46; or
the heavy chain variable region comprises: SEQ ID NO:34 and the light chain variable region comprises: SEQ ID NO:47;
the heavy chain variable region comprises: SEQ ID NO:37 and the light chain variable region comprises: SEQ ID NO:46; or
the heavy chain variable region comprises: SEQ ID NO:37 and the light chain variable region comprises: SEQ ID NO:47
the heavy chain variable region comprises: SEQ ID NO:38 and the light chain variable region comprises: SEQ ID NO:44 or
the heavy chain variable region comprises: SEQ ID NO:38 and the light chain variable region comprises: SEQ ID NO:45 or
the heavy chain variable region comprises: SEQ ID NO:39 and the light chain variable region comprises: SEQ ID NO:44 or
the heavy chain variable region comprises: SEQ ID NO:39 and the light chain variable region comprises: SEQ ID NO:45 or
the heavy chain variable region comprises: SEQ ID NO:38 and the light chain variable region comprises: SEQ ID NO:46 or
the heavy chain variable region comprises: SEQ ID NO:38 and the light chain variable region comprises: SEQ ID NO:47 or
the heavy chain variable region comprises: SEQ ID NO:39 and the light chain variable region comprises: SEQ ID NO:46 or
the heavy chain variable region comprises: SEQ ID NO:39 and the light chain variable region comprises: SEQ ID NO:47.

Any of the above combinations of heavy chain variable regions and light chain variable regions can be paired (as part of the heavy chain) with an Fc domain, e.g., of human isotype IgG1, IgG2, IgG3 or IgG4. For example, in some embodiments, the Fc domain comprises a human IgG4 Fc domain comprising one, two or all three of mutations (compared to wildtype) S228P, R409K, and/or L445P. In some embodiments, the Fc domain comprises SEQ ID NO:59.

As noted, the antibodies provided herein bind human (and in some embodiments other mammalian, e.g., such as mouse, guinea pig, pig, and rabbit) integrin αvβ8. In some embodiments, the antibodies are isolated, are chimeric (comprising at least some heterologous amino acid sequence), are labeled or covalently linked to another molecule such a cytotoxic agent or a combination thereof. In some embodiments, the antibodies specifically bind human integrin αvβ8 and block binding of a ligand to human integrin αvβ8. Exemplary ligands can include, for example, L-TGF-β and LAP.

The ability of an antibody to block αvβ8 integrin binding of a ligand can be determined by inhibition of binding of a soluble form of αvβ8 or a full-length form of αvβ8 expressed on the surface of cells to immobilized L-TGF-β or a portion thereof containing the sequence RGDL. See, e.g., Ozawa, A, et al. J Biol Chem. 291 (22): 11551-65 (2016).

For preparation and use of suitable antibodies as described herein, e.g., recombinant, monoclonal, or polyclonal antibodies, many techniques known in the art can be used (see, e.g., Kohler & Milstein, Nature 256:495-497 (1975); Kozbor et al., Immunology Today 4:72 (1983); Cole et al., pp. 77-96 in Monoclonal Antibodies and Cancer Therapy, Alan R. Liss, Inc. (1985); Coligan, Current Protocols in Immunology (1991); Harlow & Lane, Antibodies, A Laboratory Manual (1988); and Goding, Monoclonal Antibodies: Principles and Practice (2d ed. 1986)). The genes encoding the heavy and light chains of an antibody of interest can be cloned from a cell, e.g., the genes encoding a monoclonal antibody can be cloned from a hybridoma and used to produce a recombinant monoclonal antibody. Gene libraries encoding heavy and light chains of monoclonal antibodies can also be made from hybridoma or plasma cells. Random combinations of the heavy and light chain gene products generate a large pool of antibodies with different antigenic specificity (see, e.g., Kuby, Immunology (3^{rd} ed. 1997)). Techniques for the production of single chain antibodies or recombinant antibodies (U.S. Pat. Nos. 4,946,778, 4,816,567) can be adapted to produce antibodies to polypeptides of this invention. Also, transgenic mice, or other organisms such as other mammals, can be used to express humanized or human antibodies (see, e.g., U.S. Pat. Nos. 5,545,807; 5,545,806; 5,569,825; 5,625,126; 5,633,425; 5,661,016, Marks et al., Bio Technology 10:779-783 (1992); Lonberg et al., Nature 368:856-859 (1994); Morrison, Nature 368:812-13 (1994); Fishwild et al., Nature Biotechnology 14:845-51 (1996); Neuberger, Nature Biotechnology 14:826 (1996); and Lonberg & Huszar, Intern. Rev. Immunol. 13:65-93 (1995)). Alternatively, phage display technology can be used to identify antibodies and heteromeric Fab fragments that specifically bind to selected antigens (see, e.g., McCafferty et al., Nature 348:552-554 (1990); Marks et al., Biotechnology 10:779-783 (1992)). Antibodies can also be made bispecific, i.e., able to recognize two different antigens (see, e.g., WO 93/08829, Traunecker et al., EMBO J. 10:3655-3659 (1991); and Suresh et al., Methods in Enzymology 121:210 (1986)). Antibodies can also be heteroconjugates, e.g., two covalently joined antibodies, or immunotoxins (see, e.g., U.S. Pat. No. 4,676,980, WO 91/00360; WO 92/200373; and EP 03089).

Antibodies can be produced using any number of expression systems, including prokaryotic and eukaryotic expression systems. In some embodiments, the expression system is a mammalian cell expression, such as a hybridoma, or a CHO cell expression system. Many such systems are widely available from commercial suppliers. In embodiments in which an antibody comprises both a V_{H} and V_{L} region, the V_{H} and V_{L} regions may be expressed using a single vector, e.g., in a di-cistronic expression unit, or under the control of different promoters. In other embodiments, the V_{H} and V_{L} region may be expressed using separate vectors. A V_{H} or V_{L} region as described herein may optionally comprise a methionine at the N-terminus.

An antibody as described herein can also be produced in various formats, including as a Fab, a Fab′, a F(ab′)₂, a scFv, or a dAB. The antibody fragments can be obtained by a variety of methods, including, digestion of an intact antibody with an enzyme, such as pepsin (to generate (Fab′)₂ fragments) or papain (to generate Fab fragments); or de novo synthesis. Antibody fragments can also be synthesized using recombinant DNA methodology. In some embodiments, an anti-β8 antibody comprises F(ab′)₂ fragments that specifically bind β8. An antibody of the invention can also include a human constant region. See, e.g., Fundamental Immunology (Paul ed., 4d ed. 1999); Bird, et al., Science 242:423 (1988); and Huston, et al., Proc. Natl. Acad. Sci. USA 85:5879 (1988).

Methods for humanizing or primatizing non-human antibodies are also known in the art. Generally, a humanized antibody has one or more amino acid residues introduced into it from a source which is non-human. These non-human amino acid residues are often referred to as import residues, which are typically taken from an import variable domain. Humanization can be essentially performed following a number of methods including but not limited to the method of Winter and co-workers (see, e.g., Jones et al., Nature 321:522-525 (1986); Riechmann et al., Nature 332:323-327 (1988); Verhoeyen et al., Science 239:1534-1536 (1988) and Presta, Curr. Op. Struct. Biol. 2:593-596 (1992)), by substituting rodent CDRs or CDR sequences for the corresponding sequences of a human antibody. Such humanized antibodies are chimeric antibodies (U.S. Pat. No. 4,816,567), wherein substantially less than an intact human variable domain has been substituted by the corresponding sequence from a non-human species. In practice, humanized antibodies are typically human antibodies in which some CDR residues and possibly some FR residues are substituted by residues from analogous sites in rodent antibodies.

In certain embodiments the antibodies described herein have full, partial or greatly reduced effector function. In certain embodiments, the anti-αvβ8 antibodies are created to contain mutations in the hinge and Fc domains that may contain one or more modifications including but not limited to those that alter arm exchange, thermodynamic stability, complement fixation, Fc-receptor binding and/or antigen-dependent cytotoxicity (ADCC), pharmacokinetic properties or aggregation. As an example, the hinge mutation S228P, which mimics the hinge of IgG1, is known in the art to reduce arm exchange of the IgG4 isotype. As examples of mutations that change thermodynamic stability and/or pharmacokinetic properties a number of mutations increase half-life by increasing stability or binding to the neonatal Fc receptor (including but not limited to: R435H, N434A, M252Y/S254T/T256E, M428L/N434S, T252L/T253S/T254F, E294A/T307P/N434Y, T256/N378V/S383N/N434Y, E2944). As examples of mutations that reduce aggregation R409K and L445P replace IgG4 positions with corresponding amino acids in IgG1 (Namisaki, et al (2020), PLOSone, 15:3; Xu, et al (2019) Mabs, 11 (7): 1289-1299). As examples of reduced effector function antibodies described herein may be contain Fc domains with reduced effector function (e.g. IgG2 or IgG4), or IgG1, or other isotypes, with specific mutations that are known in the art to reduce effector function (including but not limited to: L235E, L234A/L235A, L234A/L235A/P329G, L234A/L235A/P329A, P331S/L234E/L235/F, G237A, D265A, D270A, E318A, K322A, P329A, D330L, A330L, P331A, E233P, G236R/L328R, or various combinations thereof). Examples of such antibodies are provided in Saunders (2019) Front Immunol 10:1296. As examples of additional modifications that affect antibody effector function, antibodies described herein can contain mutations that alter glycosylation (e.g. N297). Alternatively, glycosylation patterns can be chemically modified, or changed based on genetic modification of the producer cell, and such changes alter the functional properties of the antibody. Such cell lines commonly used in the art include the Cho.lec derivatives (described in Stanley (1989) Mol. Cell Biol. 9 (2): 377-383) or GnTIII cells (described in Davies (2001) Biotechnol Bioeng (74 (4): 288-94).

In some cases, the antibody or antibody fragment can be conjugated to another molecule, e.g., polyethylene glycol (PEGylation) or serum albumin, to provide an extended half-life in vivo. Examples of PEGylation of antibody fragments are provided in Knight et al. Platelets 15:409, 2004 (for abciximab); Pedley et al., Br. J. Cancer 70:1126, 1994 (for an anti-CEA antibody); Chapman et al., Nature Biotech. 17:780, 1999; and Humphreys, et al., Protein Eng. Des. 20:227, 2007). The antibody or antibody fragment can also be labeled, or conjugated to a therapeutic agent as described below.

The specificity of antibody binding can be defined in terms of the comparative dissociation constants (Kd) of the antibody for the target (e.g., β8) as compared to the dissociation constant with respect to the antibody and other materials in the environment or unrelated molecules in general. Typically, the Kd for the antibody with respect to the unrelated material will be at least 2-fold, 3-fold, 4-fold, 5-fold, 10-fold, 20-fold, 50-fold, 100-fold, 200-fold or higher than Kd with respect to the target.

The desired affinity for an antibody, e.g., high (pM to low nM), medium (low nM to 100 nM), or low (about 100 nM or higher), may differ depending upon whether it is being used as a diagnostic or therapeutic. For example, an antibody with medium affinity may be more successful in localizing to desired tissue as compared to one with a high affinity. Thus, antibodies having different affinities can be used for diagnostic and therapeutic applications.

A targeting moiety will typically bind with a Kd of less than about 1000 nM, e.g., less than 250, 100, 50, 20 or lower nM. In some embodiments, the Kd of the affinity agent is less than 15, 10, 5, or 1 nM. In some embodiments, the Kd is 1-100 nM, 0.1-50 nM, 0.1-10 nM, or 1-20 nM. The value of the dissociation constant (Kd) can be determined by well-known methods, and can be computed even for complex mixtures by methods as disclosed, e.g., in Caceci et al., Byte (1984) 9:340-362.

Affinity of an antibody, or any targeting agent, for a target can be determined according to methods known in the art, e.g., as reviewed in Ernst et al. Determination of Equilibrium Dissociation Constants, Therapeutic Monoclonal Antibodies (Wiley & Sons ed. 2009).

Quantitative ELISA, and similar array-based affinity methods can be used. ELISA (Enzyme linked immunosorbent signaling assay) is an antibody-based method. In some cases, an antibody specific for target of interest is affixed to a substrate, and contacted with a sample suspected of containing the target. The surface is then washed to remove unbound substances. Target binding can be detected in a variety of ways, e.g., using a second step with a labeled antibody, direct labeling of the target, or labeling of the primary antibody with a label that is detectable upon antigen binding. In some cases, the antigen is affixed to the substrate (e.g., using a substrate with high affinity for proteins, or a Strepavidin-biotin interaction) and detected using a labeled antibody (or other targeting moiety). Several permutations of the original ELISA methods have been developed and are known in the art (see Lequin (2005) Clin. Chem. 51:2415-18 for a review).

The Kd, Kon, and Koff can also be determined using surface plasmon resonance (SPR), e.g., as measured by using a Biacore T100 system or a solution based protein interaction assay such as a kinetic exclusion assay (e.g., KinExA®). SPR techniques are reviewed, e.g., in Hahnfeld et al. Determination of Kinetic Data Using SPR Biosensors, Molecular Diagnosis of Infectious Diseases (2004). In a typical SPR experiment, one interactant (target or targeting agent) is immobilized on an SPR-active, gold-coated glass slide in a flow cell, and a sample containing the other interactant is introduced to flow across the surface. When light of a given frequency is shined on the surface, the changes to the optical reflectivity of the gold indicate binding, and the kinetics of binding. Kinetic exclusion assays are the preferred method to determine affinity since neither antibody nor antigen requires immobilization, unless indicated otherwise. This technique is described in, e.g. Darling et al., Assay and Drug Development Technologies Vol. 2, number 6 647-657 (2004).

Binding affinity can also be determined by anchoring a biotinylated interactant to a streptavidin (SA) sensor chip. The other interactant is then contacted with the chip and detected, e.g., as described in Abdessamad et al. (2002) Nuc. Acids Res. 30: e45.

Also provided are polynucleotides (e.g., DNA or RNA) encoding the antibodies described herein, or binding fragments thereof comprising at least heavy chain or light chain CDRs or both, e.g., polynucleotides, expression cassettes (e.g., a promoter linked to a coding sequence), or expression vectors encoding heavy or light chain variable regions or segments comprising the complementary determining regions as described herein. In some embodiments, the polynucleotide sequence is optimized for expression, e.g., optimized for mammalian expression or optimized for expression in a particular cell type.

### Methods of Treatment

The anti-αvβ8 antibodies described herein (including αvβ8 binding fragments thereof, labeled antibodies, immunoconjugates, pharmaceutical compositions, etc.) as well as antibodies that bind both αvβ8 and αvβ6 as described herein or binding fragments thereof can be used to detect, treat, ameliorate, or prevent a variety of diseases mediated by avb8. Such diseases include, but are not limited to cancer, fibrosis and inflammatory diseases. In some embodiments, the disease is selected from chronic obstructive pulmonary disease (COPD) and asthma, inflammatory bowel disease, inflammatory brain autoimmune disease, multiple sclerosis, a demyelinating disease (e.g., transverse myelitis, Devic's disease, Guillain-Barré syndrome), neuroinflammation, kidney disease, or glioma, arthritis, fibrotic disorders, such as airway fibrosis, idiopathic pulmonary fibrosis, non-specific interstitial pneumonia, post-infectious lung fibrosis, diffuse alveolar damage, collagen-vascular disease associated lung fibrosis, drug-induced lung fibrosis, silicosis, asbestos-related lung fibrosis, respiratory bronchiolitis, respiratory bronchiolitis interstitial lung disease, desquamative interstitial fibrosis, cryptogenic organizing pneumonia, chronic hypersensitivity pneumonia, drug-related lung or hepatic fibrosis, renal fibrosis (e.g. associated with diabetes, IgA nephropathy, focal segmental glomerulosclerosis, interstitial fibrosis), and liver fibrosis (e.g., induced by alcohol, drug use, steatohepatitis, viral infection (e.g., hepatitis B or C), cholestasis, etc., and cancer, including but not limited to lung cancer, adenocarcinoma, squamous carcinoma, breast carcinoma, and cancer growth and metastasis. Accordingly, the antibodies and pharmaceutical compositions described herein can be administered to a human having or suspected of having one of the above-listed diseases in an appropriate dosage to ameliorate or treat one of the disease or at least one symptom thereof.

Without intending to limit the scope of the invention, in some embodiments it is believed that antibodies described herein function in part by triggering an increase in MHCII expression in antigen presenting cells, inhibiting the differentiation and function of Treg cells, destabilizing tumor vessel differentiation, or altering chemokine secretion. In another aspect, the antibodies described herein can target the integrin αvβ8 in the tumor microenvironment including but not limited to the tumor cells themselves, stromal cells including tumor associated fibroblasts, or certain immune cell subsets including, T-cells, Treg, or myeloid cells. In another aspect targeting αvβ8 induces cytotoxicity of cells in the tumor microenvironment including but not limited to the tumor cells themselves, stromal cells including tumor associated fibroblasts, or certain immune cell subsets including, Treg, or myeloid cells either by antibody effector function, immunoconjugate, chemical modification or pharmaceutical composition. In some embodiments, targeting αvβ8 selectively inhibits activation of TGF-β1 and TGF-β3 when expressed on the surface of cells, incorporated into the extracellular matrix or soluble in the tumor microenvironment, which affects proliferation, survival or differentiation of cells in the tumor microenvironment including but not limited to the tumor cells themselves, stromal cells including tumor associated fibroblasts, or certain immune cell subsets including, T-cells, Treg or myeloid cells.

In some embodiments, the cancer cells themselves express β8. For example, in some embodiments, the cancer cells are bladder, colorectal, glioblastoma, gynecologic (endometrial, uterine, stromal, endocervical, cervical, fallopian or ovarian), liver (hepatoma, cholangial), head and neck, kidney (clear cell, papillary, chromophobe), lung, skin, pancreas, sarcoma, breast, gastric, esophageal, urothelial, prostate, or CNS cancer cells. In other embodiments, the cancer cells do not express β8, or express β8 at low levels (e.g., see examples), and in some embodiments, other cells in the tumor microenvironment, such as cancer-associated fibroblasts, or specific immune cells including but not limited to Treg or myeloid cells, express αvβ8. In another aspect, the antibodies described herein can be combined with other therapies including, checkpoint inhibitors (i.e., anti-PD-1, anti-PD-L1, anti-CTLA4, anti-LAG3, anti-TIM3, anti-TIGIT), chemotherapy, radiation therapy, cell therapy including chimeric antigen receptor therapy, and other immunomodulator agents. Such combination therapies can be applied before, during or after administration of the integrin β8 antibodies described herein. For example, in some embodiments, the therapy (which can include but is not limited to anti-PD-1 treatment) reduces expression of β8 in a sample from the individual and then the anti-β8 antibodies as described herein are administered. As shown in the examples this can be particularly efficacious. In some other embodiments, the cancer is characterized by elevated αvβ8 expression, elevated expression of TGF-β response genes, an αvβ8 expression signature, with or without infiltration of regulatory T cells, immunosuppressive myeloid-derived suppressor cells, or tumor-associated macrophages.

In some embodiments, the antibodies described herein can be expressed as scFvs on T-cells as chimeric-antigen receptors. T-cells expressing such CARs can be used, for the treatment of cancers that express αvβ8, for example but not limited to, breast, lung, colon, gastric, head and neck, esophageal, ovarian and other gynecologic malignancies, urothelial, renal, skin, prostate, pancreatic, melanoma, or CNS malignancies or other cancers described herein.

In some embodiments, the individual has a cancer, e.g., solid tumor, and an immune desert phenotype (see, e.g., Hegde et al., Immunity 52, Jan. 14, 2020 describing immune deserts), i.e., showing no immune cell infiltration. As described in the Examples, it has been found that such individuals are responsive to the antibodies described herein. Thus, in some embodiments, methods of treating an individual having cancer lacking immune cell infiltration with an anti-integrin αvβ8 antibody as described herein is provided. Tumors in an individual lacking immune cell infiltration,” as used herein is as defined in Combes, et al (2022) Cell, 5, 420:123-127).

Moreover, the anti-αvβ8 antibodies described herein (including αvβ8 binding fragments thereof, labeled antibodies, immunoconjugates, pharmaceutical compositions, etc.) can be used to treat, ameliorate, or prevent viral infections (e.g., by stimulating an immune response). Exemplary viral infections include but are not limited to hepatitis A, B (HBV), and C

(HCV), herpes simplex virus (e.g., HSVI, HSVII), HIV, and influenza infections, all of which are enhanced by Treg-mediated immune suppression (Keynan, Y, et al., Clin Infect Dis. 2008 Apr. 1; 46 (7): 1046-52.

Also provided are pharmaceutical compositions comprising the present anti-αvβ8 antibodies or antigen-binding molecules as well as antibodies that bind both αvβ8 and αvβ6 as described herein or binding fragments thereof, either of which can be formulated together with a pharmaceutically acceptable carrier. The compositions can additionally contain other therapeutic agents that are suitable for treating or preventing a given disorder. Pharmaceutically carriers can enhance or stabilize the composition, or to facilitate preparation of the composition. Pharmaceutically acceptable carriers include solvents, dispersion media, coatings, antibacterial and antifungal agents, isotonic and absorption delaying agents, and the like that are physiologically compatible.

A pharmaceutical composition as described herein can be administered by a variety of methods known in the art. The route and/or mode of administration vary depending upon the desired results. It is preferred that administration be intravenous, intramuscular, intraperitoneal, or subcutaneous, or administered proximal to the site of the target. The pharmaceutically acceptable carrier should be suitable for intravenous, intramuscular, subcutaneous, parenteral, intranasal, inhalational, spinal or epidermal administration (e.g., by injection or infusion). Depending on the route of administration, the active compound, i.e., antibody, may be coated in a material to protect the compound from the action of acids and other natural conditions that may inactivate the compound.

The antibodies, alone or in combination with other suitable components, can be made into aerosol formulations (i.e., they can be “nebulized”) to be administered via inhalation. Aerosol formulations can be placed into pressurized acceptable propellants, such as dichlorodifluoromethane, propane, nitrogen, and the like.

In some embodiments, the composition is sterile and fluid. Proper fluidity can be maintained, for example, by use of coating such as lecithin, by maintenance of required particle size in the case of dispersion and by use of surfactants. In many cases, it is preferable to include isotonic agents, for example, sugars, polyalcohols such as mannitol or sorbitol, and sodium chloride in the composition. Long-term absorption of the injectable compositions can be brought about by including in the composition an agent which delays absorption, for example, aluminum monostearate or gelatin.

Pharmaceutical compositions of the invention can be prepared in accordance with methods well known and routinely practiced in the art. Pharmaceutically acceptable carriers are determined in part by the particular composition being administered, as well as by the particular method used to administer the composition. Accordingly, there is a wide variety of suitable formulations of pharmaceutical compositions of the present invention. Applicable methods for formulating the antibodies and determining appropriate dosing and scheduling can be found, for example, in Remington: The Science and Practice of Pharmacy, 21st Ed., University of the Sciences in Philadelphia, Eds., Lippincott Williams & Wilkins (2005); and in Martindale: The Complete Drug Reference, Sweetman, 2005, London: Pharmaceutical Press., and in Martindale, Martindale: The Extra Pharmacopoeia, 31st Edition., 1996, Amer Pharmaceutical Assn, and Sustained and Controlled Release Drug Delivery Systems, J.R. Robinson, ed., Marcel Dekker, Inc., New York, 1978, each of which are hereby incorporated herein by reference. Pharmaceutical compositions are preferably manufactured under GMP conditions. Typically, a therapeutically effective dose or efficacious dose of the anti-αvβ8 antibody is employed in the pharmaceutical compositions of the invention. The anti-αvβ8 antibodies are formulated into pharmaceutically acceptable dosage forms by conventional methods known to those of skill in the art. Dosage regimens are adjusted to provide the desired response (e.g., a therapeutic response). In determining a therapeutically or prophylactically effective dose, a low dose can be administered and then incrementally increased until a desired response is achieved with minimal or no undesired side effects. For example, a single bolus may be administered, several divided doses may be administered over time or the dose may be proportionally reduced or increased as indicated by the exigencies of the therapeutic situation. It is especially advantageous to formulate parenteral compositions in dosage unit form for ease of administration and uniformity of dosage. Dosage unit form as used herein refers to physically discrete units suited as unitary dosages for the subjects to be treated; each unit contains a predetermined quantity of active compound calculated to produce the desired therapeutic effect in association with the required pharmaceutical carrier.

Actual dosage levels of the active ingredients in the pharmaceutical compositions of the present invention can be varied so as to obtain an amount of the active ingredient which is effective to achieve the desired therapeutic response for a particular patient, composition, and mode of administration, without being toxic to the patient. The selected dosage level depends upon a variety of pharmacokinetic factors including the activity of the particular compositions of the present invention employed, or the ester, salt or amide thereof, the route of administration, the time of administration, the rate of excretion of the particular compound being employed, the duration of the treatment, other drugs, compounds and/or materials used in combination with the particular compositions employed, the age, sex, weight, condition, general health and prior medical history of the patient being treated, and like factors.

In some embodiments, the pharmacological compositions comprise a mixture of the anti-αvβ8 antibody or antigen binding molecule (e.g. that blocks ligand binding or blocks activation by ligand binding) and a second pharmacological agent. Without intending to limit the invention, it is noted that the inventors have found that thymic stromal lymphopoietin (TSLP) is an inducer of viral clearance in a mouse model of acute and chronic HBV and thus is useful to combine TSLP with an αvβ8 antibody for anti-viral treatments. Moreover, the inventors have found that OX40 agonists are effective in stimulating an immune response to HBV in combination with an αvβ8 antibody.

As an alternative to mixing the anti-αvβ8 antibody and second pharmacological agent in a pharmacological composition, the anti-αvβ8 antibody and second pharmacological agent can be separately administered to the human in need thereof within a time frame (e.g., within 3, 2, o 1 day or within 24, 13, 6, or 3 hours of each other).

In some embodiments, the second pharmacological agent can comprise a chemotherapeutic agent, radiation, cryo therapy, immunosuppressive therapy, T-cells bearing chimeric antigen receptors, an immunostimulatory agent, an immunomodulator with either or both pro- and anti-inflammatory properties, an immune checkpoint inhibitors. Exemplary the immune checkpoint inhibitors can be selected from the group consisting of: an anti-PD1 antibody, an anti-PD-L1 antibody, an anti-CTLA-4 antibody, an anti-LAG-3 anti-body, an anti-TIGIT antibody, or an anti-TIM3 antibody. In some embodiments, the second pharmaceutical agent is an immune cell expressing a chimeric antigen receptor. In some embodiments, the anti-αvβ8 antibody inhibits TGF-beta activation thereby enhancing cytotoxic immune cell responses of T-cells expressing the chimeric antigen receptor. In some embodiments, the T-cell expressing the chimeric antigen targets -αvβ8-expressing tumor cells or -αvβ8-expressing regulatory T-cells.

### Diagnostic Compositions and Applications

Integrin αvβ8 is expressed, for example, on fibroblasts, stellate cells, chondrocytes, activated macrophages and subsets of T and B-cells. Integrin αvβ8 is increased in expression in fibroblasts in COPD and pulmonary fibrosis, and can be used as a surrogate marker for increased fibroblast cell mass. Thus the presently disclosed antibodies can be broadly applicable to bioimaging strategies to detect fibroinflammatory processes. The presently described therapeutic and diagnostic antibodies can be applied to: inflammatory bowel disease (IBD), chronic obstructive pulmonary disease (COPD), asthma, arthritis, a hepatic fibroinflammatory disorder, alcohol induced liver injury, non-alcoholic steatohepatitis (NASH), viral hepatitis, and primary biliary cirrhosis (PBC), graft rejection after liver transplantation, autoimmune hepatitis, an autoimmune disorder, lupus erythematosus, scleroderma, dermatomyositis, bullous pemphigoid, pemphigus vulgaris, a pulmonary fibrotic disorder, an inflammatory brain autoimmune disease, multiple sclerosis, a demyelinating disease, neuroinflammation, kidney disease, glomerulonephritis, hepatocellular carcinoma (HCC), adenocarcinoma, squamous carcinoma, glioma, melanoma, prostate, ovarian, uterine and breast carcinoma.

β8 and PD-L1 expression inversely correlate. Thus, anti-αvβ8 antibodies described herein can be used as a marker for PD-L1 expression and optionally for selecting individuals most likely to benefit from anti-αvβ8 treatment.

Moreover, as described in the Examples, in some embodiments, improved efficacy of the αvβ8 antibodies described herein was observed in individuals having low expression of β8. For example, for cancer treatment it has been found that cancers expressing lower levels of β8 are most responsive to the antibodies described herein. Accordingly, in some embodiments, a sample from an individual is assayed for expression of an itgb8 transcript or β8 protein and depending on the expression level, an αvβ8 antibody as described herein is administered to the individual. Any method of detection of transcripts or proteins can be used. For example, immunostaining such as immunohistochemical methods can be used to detect β8 in a sample from an individual. Methods of detection of transcripts can comprise many techniques, for example quantitative RT-PCR, sequencing, gene-array, or oligonucleotide probes.

Anti-αvβ8 antibodies described herein (including αvβ8 binding fragments thereof, affinity matured variants, or scFvs) can be used for diagnosis, either in vivo or in vitro (e.g., using a biological sample obtained from an individual).

When used for detection or diagnosis, the antibody is typically conjugated or otherwise associated with a detectable label. The association can be direct e.g., a covalent bond, or indirect, e.g., using a secondary binding agent, chelator, or linker.

A labeled antibody can be provided to an individual to determine the applicability of an intended therapy. For example, a labeled antibody may be used to detect the integrin β8 density within a diseased area. For therapies intended to target TGFβ or αvβ8 activity (to reduce TGFβ or αvβ8 activity), the density of β8 is typically high relative to non-diseased tissue. A labeled antibody can also indicate that the diseased area is accessible for therapy. Patients can thus be selected for therapy based on imaging results. Anatomical characterization, such as determining the precise boundaries of a cancer, can be accomplished using standard imaging techniques (e.g., CT scanning, MRI, PET scanning, etc.). Such in vivo methods can be carried out using any of the presently disclosed antibodies.

Any of the presently disclosed antibodies can also be used for in vitro diagnostic or monitoring methods, e.g., using cells or tissue from a patient sample. In some embodiments, labeled antibodies described herein can bind fixed cells as well as non-fixed cells.

In some embodiments, the diagnostic antibody is a single-chain variable fragment (scFv). Intact antibodies (e.g., IgG) can be used for radioimmunotherapy or targeted delivery of therapeutic agents because they exhibit high uptake and retention. In some cases, the persistence in circulation of intact mAbs can result in high background (Olafsen et al. (2012) Tumour Biol. 33:669-77; Cai et al. (2007) J Nucl Med. 48:304-10). ScFvs, typically with a molecular mass of ˜25 kD, are rapidly excreted by the kidneys, but are monovalent and can have lower affinity. The issues of monovalency can be overcome with advanced antibody engineering (as shown herein), where affinities can be improved to the low nM to pM range. Such antibodies have short enough half-lives to be useful as imaging agents and have suitable binding characteristics for tissue targeting (Cortez-Retamozo et al. (2004) Cancer Res. 64:2853-7).

A diagnostic agent comprising an antibody described herein can include any diagnostic agent known in the art, as provided, for example, in the following references: Armstrong et al., Diagnostic Imaging, 5^{th} Ed., Blackwell Publishing (2004); Torchilin, V. P., Ed., Targeted Delivery of Imaging Agents, CRC Press (1995); Vallabhajosula, S., Molecular Imaging: Radiopharmaceuticals for PET and SPECT, Springer (2009). The terms “detectable agent,” “detectable moiety,” “label,” “imaging agent,” and like terms are used synonymously herein. A diagnostic agent can be detected by a variety of ways, including as an agent providing and/or enhancing a detectable signal. Detectable signals include, but are not limited to, gamma-emitting, radioactive, echogenic, optical, fluorescent, absorptive, magnetic, or tomography signals. Techniques for imaging the diagnostic agent can include, but are not limited to, single photon emission computed tomography (SPECT), magnetic resonance imaging (MRI), optical imaging, positron emission tomography (PET), computed tomography (CT), x-ray imaging, gamma ray imaging, and the like. PET is particularly sensitive and quantitative, and thus valuable for characterizing fibrotic processes in vivo (Olafsen et al. (2012) Tumour Biol. 33:669-77; Cai et al. (2007) J Nucl Med. 48:304-10). This is useful beyond a companion diagnostic and would be generally useful to diagnose, clinically stage and follow fibrotic patients during any treatment regimen.

A radioisotope can be incorporated into the diagnostic agents described herein and can include radionuclides that emit gamma rays, positrons, beta and alpha particles, and X-rays. Suitable radionuclides include but are not limited to ²²⁵Ac, ⁷²As, ²¹¹At, ¹¹B, ¹²⁸Ba, ²¹²Bi, ⁷⁵Br, ⁷⁷Br, ¹⁴C, ¹⁰⁹Cd, ⁶²Cu, ⁶⁴Cu, ⁶⁷Cu, ¹⁸F, ⁶⁷Ga, ⁶⁸Ga, ³H, ¹⁶⁶Ho, ¹²³I, ¹²⁴I, ¹²⁵I, ¹³⁰I, ¹³¹I, ¹¹¹In, ¹⁷⁷Lu, ¹³N, ¹⁵O, ³²P, ³³P, ²¹²Pb, ¹⁰³Pd, ¹⁸⁶Re, ¹⁸⁸Re, ⁴⁷Sc, ¹⁵³Sm, ⁸⁹Sr, ^{99m}Tc, ⁸⁸Y and ⁹⁰Y. In certain embodiments, radioactive agents can include ¹¹¹In-DTPA, ^{99m}Tc(CO)₃-DTPA, ^{99m}Tc(CO)₃-ENPy2, ^{62/64/67}Cu-TETA, ^{99m}Tc (CO)₃-IDA, and ^{99m}Tc(CO)₃triamines (cyclic or linear). In other embodiments, the agents can include DOTA and its various analogs with ¹¹¹In, ¹⁷⁷Lu, ¹⁵³Sm, ^{88/90}Y, ^{62/64/67}Cu, or ^{67/68}Ga. In some embodiments, a nanoparticle can be labeled by incorporation of lipids attached to chelates, such as DTPA-lipid, as provided in the following references: Phillips et al., Wiley Interdisciplinary Reviews: Nanomedicine and Nanobiotechnology, 1 (1): 69-83 (2008); Torchilin, V.P. & Weissig, V., Eds. Liposomes 2nd Ed.: Oxford Univ. Press (2003); Elbayoumi, T.A. & Torchilin, V.P., Eur. J. Nucl. Med. Mol. Imaging 33:1196-1205 (2006); Mougin-Degraef, M. et al., Int'l J. Pharmaceutics 344:110-117 (2007).

In some embodiments, a diagnostic agent can include chelators that bind, e.g., to metal ions to be used for a variety of diagnostic imaging techniques. Exemplary chelators include but are not limited to ethylenediaminetetraacetic acid (EDTA), [4-(1,4,8, 11-tetraazacyclotetradec-1-yl)methyl] benzoic acid (CPTA), Cyclohexanediaminetetraacetic acid (CDTA), ethylenebis(oxyethylenenitrilo)tetraacetic acid (EGTA), diethylenetriaminepentaacetic acid (DTPA), citric acid, hydroxyethyl ethylenediamine triacetic acid (HEDTA), iminodiacetic acid (IDA), triethylene tetraamine hexaacetic acid (TTHA), 1,4,7,10-tetraazacyclododecane-1,4,7,10-tetra(methylene phosphonic acid) (DOTP), 1,4,8,11-tetraazacyclotetradecane-1,4,8,11-tetraacetic acid (TETA), 1,4,7,10-tetraazacyclododecane-1,4,7,10-tetraacetic acid (DOTA), N1,N1-bis(pyridin-2-ylmethyl) ethane-1,2-diamine (ENPy2) and derivatives thereof.

In some embodiments, the diagnostic agent can be associated with a secondary binding ligand or to an enzyme (an enzyme tag) that will generate a colored product upon contact with a chromogenic substrate. Examples of suitable enzymes include urease, alkaline phosphatase, (horseradish) hydrogen peroxidase and glucose oxidase. Secondary binding ligands include, e.g., biotin and avidin or streptavidin compounds as known in the art.

In some embodiments, the diagnostic agents can include optical agents such as fluorescent agents, phosphorescent agents, chemiluminescent agents, and the like. Numerous agents (e.g., dyes, probes, labels, or indicators) are known in the art and can be used in the present invention. (See, e.g., Invitrogen, The Handbook—A Guide to Fluorescent Probes and Labeling Technologies, Tenth Edition (2005)). Fluorescent agents can include a variety of organic and/or inorganic small molecules or a variety of fluorescent proteins and derivatives thereof. For example, fluorescent agents can include but are not limited to cyanines, phthalocyanines, porphyrins, indocyanines, rhodamines, phenoxazines, phenylxanthenes, phenothiazines, phenoselenazines, fluoresceins, benzoporphyrins, squaraines, dipyrrolo pyrimidones, tetracenes, quinolines, pyrazines, corrins, croconiums, acridones, phenanthridines, rhodamines, acridines, anthraquinones, chalcogenopyrylium analogues, chlorins, naphthalocyanines, methine dyes, indolenium dyes, azo compounds, azulenes, azaazulenes, triphenyl methane dyes, indoles, benzoindoles, indocarbocyanines, benzoindocarbocyanines, and BODIPY™ derivatives.

In some embodiments, the heavy and light chain variable region combinations described herein are integrated into a chimeric antigen receptor (CAR) polypeptide as the extracellular binding domain, which can be expressed in an immune cell. The heavy and light chain variable region combinations can be in any appropriate format for example as a scFv.

Chimeric antigen receptors (CARs) are recombinant receptor constructs comprising an extracellular antigen-binding domain (e.g., a nanobody) joined to a transmembrane domain, and further linked to an intracellular signaling domain (e.g., an intracellular T cell signaling domain of a T cell receptor) that transduces a signal to elicit a function. In certain embodiments, immune cells (e.g., T cells or natural killer (NK) cells) are genetically modified to express CARs that comprise one or more of the heavy and light chain variable regions described herein and have the functionality of effector cells (e.g., cytotoxic and/or memory functions of T cells or NK cells).

In a standard CAR, the components include an extracellular targeting domain, a transmembrane domain and intracellular signaling/activation domain, which are typically linearly constructed as a single fusion protein. In the present invention, the extracellular region comprises an anti-αvβ8 binding sequence as described herein. The “transmembrane domain” is the portion of the CAR that links the extracellular binding portion and intracellular signaling domain and anchors the CAR to the plasma membrane of the host cell that is modified to express the CAR, e.g., the plasma membrane of an immune effector cell. The intracellular region may contain a signaling domain of TCR complex, and/or one or more costimulatory signaling domains, such as those from CD28, 4-1BB (CD137) and OX-40 (CD134). For example, a “first-generation CAR” generally has a CD3-zeta signaling domain. Additional costimulatory intracellular domains may also be introduced (e.g., second and third generation CARS) and further domains including homing and suicide domains may be included in CAR constructs. CAR components are further described below.

### Extracellular Domain (e.g., Nanobody Domain)

A chimeric antigen receptor of the present disclosure comprises an extracellular antigen-binding domain that comprises, for example, an scFv comprising a heavy and light chain variable region combination as described herein, e.g., having three heavy chain CDRs and three light chain CDRs, each in a framework (FR) sequence.

A CAR construct encoding a CAR can also comprise a sequence that encodes a signal peptide to target the extracellular domain to the cell surface.

### Hinge Domain

In some embodiments, the CAR may one or more hinge domains that link the antigen binding domain comprising an anti-αvβ8 antibody as described herein and the transmembrane domain for positioning the antigen binding domain. Such a hinge domain may be derived either from a natural, synthetic, semi-synthetic, or recombinant source. The hinge domain can include the amino acid sequence of a naturally occurring immunoglobulin hinge region, e.g., a naturally occurring human immunglobuline hinge region, or an altered immunoglobulin hinge region. Illustrative hinge domains suitable for use in the CARs described herein include the hinge region derived from the extracellular regions of type 1 membrane proteins such as CD8 alpha, CD4, CD28, PD1, CD 152, and CD7, which may be wild-type hinge regions from these molecules or may be altered.

### Transmembrane Domain

Any transmembrane suitable for use in a CAR construct may be employed. Such transmembrane domains, include, but are not limited to, all or part of the transmembrane domain of the alpha, beta or zeta chain of the T-cell receptor, CD28, CD27, CD3 epsilon, CD45, CD4, CD5, CD8, CD9, CD16, CD22, CD33, CD37, CD64, CD80, CD86, CD134, CD137, CD154. In some embodiments, a transmembrane domain may include at least the transmembrane region(s) of, e.g., KIRDS2, OX40, CD2, CD27, LFA-1 (CD 11a, CD18), ICOS (CD278), 4-1BB (CD137), GITR, CD40, BAFFR, HVEM (LIGHTR), SLAMF7, NKp80 (KLRF1), NKp44, NKp30, NKp46, CD160, CD19, IL2R beta, IL2R gamma, IL7R a, ITGA1, VLA1, CD49a, ITGA4, IA4, CD49D, ITGA6, VLA-6, CD49f, ITGAD, CD11d, ITGAE, CD103, ITGAL, CD11a, LFA-1, ITGAM, CDI 1b, ITGAX, CD11c, ITGB1, CD29, ITGB2, CD 18, LFA-1, ITGB7, TNFR2, DNAM1 (CD226), SLAMF4 (CD244, 2B4), CD84, CD96 (Tactile), CEACAM1, CRT AM, Ly9 (CD229), CD160 (BY55), PSGL1, CD100, (SEMA4D), SLAMF6 (NTB-A, Ly108), SLAM (SLAMF1, CD150, IPO-3), BLAME, (SLAMF8), SELPLG (CD162), LTBR, PAG/Cbp, NKG2D, or NKG2C.

A transmembrane domain incorporated into a CAR construct may be derived either from a natural, synthetic, semi-synthetic, or recombinant source.

### Intracellullar Signaling Domain

A CAR construct of the present disclosure includes one or more intracellular signaling domains, also referred to herein as co-stimulatory domains, or cytoplasmic domains that activate or otherwise modulate an immune cell, (e.g., a T lymphocyte or NK cell). The intracellular signaling domain is generally responsible for activation of at least one of the normal effector functions of the immune cell in which the CAR has been introduced. In one embodiment, a co-stimulatory domain is used that increases CAR immune T cell cytokine production. In another embodiment, a co-stimulatory domain is used that facilitates immune cell (e.g., T cell) replication. In still another embodiment, a co-stimulatory domain is used that prevents CAR immune cell (e.g., T cell) exhaustion. In another embodiment, a co-stimulatory domain is used that increases immune cell (e.g., T cell) antitumor activity. In still a further embodiment, a co-stimulatory domain is used that enhances survival of CAR immune cells (e.g., T cells) (e.g., post-infusion into patients).

Examples of intracellular signaling domains for use in a CAR include the cytoplasmic sequences of the T cell receptor (TCR) and co-receptors that act in concert to initiate signal transduction following antigen receptor engagement, as well as any derivative or variant of these sequences and any recombinant sequence that has the same functional capability.

A primary signaling domain regulates primary activation of the TCR complex either in a stimulatory way, or in an inhibitory way. Primary intracellular signaling domains that act in a stimulatory manner may contain signaling motifs which are known as immunoreceptor tyrosine-based activation motifs or ITAMs.

Examples of ITAMs containing primary intracellular signaling domains include those of CD3 zeta, common FcR gamma, Fc gamma Rlla, FcR beta (Fc Epsilon Rib), CD3 gamma, CD3 delta, CD3 epsilon, CD79a, CD79b, DAP10, and DAP12. In one embodiment, a CAR comprises an intracellular signaling domain, e.g., a primary signaling domain of CD3-zeta.

An intracellular signaling domain of a CAR can comprise a primary intracellular signaling domain only, or may comprise additional desired intracellular signaling domain(s) useful in the context of a CAR as described herein. For example, the intracellular signaling domain of the CAR can comprise a CD3 zeta chain portion and a costimulatory signaling domain. The costimulatory signaling domain refers to a portion of the CAR comprising the intracellular domain of a costimulatory molecule. A costimulatory molecule is a cell surface molecule other than an antigen receptor or its ligands that is required for an efficient response of lymphocytes to an antigen. Examples of such molecules include CD27, CD28, 4-1BB (CD137), OX40, CD30, CD40, PD-1, ICOS, lymphocyte function-associated antigen-1 (LFA-1), CD2, CD7, LIGHT, NKG2C, B7-H3, and a ligand that binds to CD83, and the like. For example, CD27 costimulation has been demonstrated to enhance expansion, effector function, and survival of human CART cells in vitro and augments human T cell persistence and antitumor activity in vivo (Song et al. Blood. 2012; 119 (3): 696-706). Further examples of such costimulatory molecules include CDS, ICAM-1, GITR, BAFFR, HVEM (LIGHTR), SLAMF7, NKp80 (KLRF1), NKp44, NKp30, NKp46, CD 160, CD 19, CD4, CD8alpha, CD8beta, IL2R beta, IL2R gamma, IL7R alpha, ITGA4, VLA1, CD49a, ITGA4, IA4, CD49D, ITGA6, VLA-6, CD49f, ITGAD, CD11d, ITGAE, CD103, ITGAL, CD11a, LFA-1, ITGAM, CD11b, ITGAX, CD11c, ITGB 1, CD29, ITGB2, CD18, LFA-1, ITGB7, TNFR2, TRANCE/RANKL, DNAM1 (CD226), SLAMF4 (CD244, 2B4), CD84, CD96 (Tactile), NKG2D, CEACAM1, CRTAM, Ly9 (CD229), CD160 (BY55), PSGL1, CD100 (SEMA4D), CD69, SLAMF6 (NTB-A, Ly108), SLAM, (SLAMF1, CD150, IPO-3), BLAME (SLAMF8), SELPLG (CD162), LTBR, LAT, GADS, SLP-76, PAG/Cbp, and CD 19a.

In some embodiments, a CAR may be designed as an inducible CAR, or may otherwise comprise a mechanisms for reversibly expressing the CAR, or controlling CAR activity to largely restrict it to a desired environment. Thus, for example, in some embodiments, the CAR-expressing cell uses a split CAR. The split CAR approach is described in more detail in publications WO2014/055442 and WO2014/055657. Briefly, a split CAR system comprises a cell expressing a first CAR having a first antigen binding domain and a costimulatory domain (e.g., 41BB), and the cell also expresses a second CAR having a second antigen binding domain and an intracellular signaling domain (e.g., CD3 zeta). When the cell encounters the first antigen, the costimulatory domain is activated, and the cell proliferates. When the cell encounters the second antigen, the intracellular signaling domain is activated and cell-killing activity begins. Thus, the CAR-expressing cell is only fully activated in the presence of both antigens.

The following examples are offered to illustrate, but not to limit the claimed invention.

### Example 1. Comparative Efficacy of Anti-αvβ8 and Anti-β8 Antibodies

Current methods in preclinical and clinical development of inhibiting integrin αvβ8-mediated TGF-β activation include both large and small molecules. We have used a structural approach to understand the features of αvβ8-binding to TGF-β that can be exploited for therapeutic development. A large binding interface is formed between the Arg-Gly-Asp (RGD) containing integrin binding loop of the LAP of TGF-β and the complex binding pocket of αvβ8. The RGD loop of TGF-β invaginates deep into this pocket created by the αv-head domain at one edge and the tip of the β8-BI domain on the other. A large flexible loop, the specificity determining loop-2 (SDL-2) of the β8 subunit connects these two edges and forms a “proximal” binding pocket formed between the αv-subunit and β8-subunit SDL2, and a hydrophobic pocket with the tip of the β8-βI domain, the later which moves inward to increase interaction with the Asp of RGD. The former pocket creates a broad docking surface for the Arg of RGD, as well as a promiscuous docking surface for the second Arg of the TGF-β3 RGD binding loop (SEQ ID NO: 49). This shows that there are multiple mechanisms by which TGF-β, which is itself highly conformationally dynamic, can initiate binding to αvβ8. The hydrophobic pocket formed by the β8 SDL2 (as well as the β6 SDL2 loop) is also dynamic, as it only is only formed in the TGF-β bound and not the apo state. The formation of this hydrophobic binding pocket facilitates formation of an α-helix of the +1 to +4 residues located just C-terminal to the RGD site of the TGF-β binding loops (SEQ ID NO 49, 51). This later helix is involved in high-affinity TGF-β binding to αvβ8. These data indicate the complex and dynamic requirements for effectively blocking binding of the RGD loop of TGF-β1 and -β3 with αvβ8. Further, adding to the binding interaction, the LAP of TGF-β1 makes additional contacts with αvβ8 outside of the RGD binding loop creating a binding interface of approximately 970 Å² (Campbell, et al (2020) Cell 180 (3): 490-501). Therefore, the most effective inhibitors to αvβ8 have plasticity to accommodate the proximal and hydrophobic binding pockets formed by SDL2, as well as a large binding interface to block additional interactions between TGF-β1 and -β3 and αvβ8.

Antibodies containing flexible CDR binding loops are ideally suited to conform to the dynamic and complex binding pocket of αvβ8 to inhibit binding interactions with the LAP of TGF-β. We sought to assess the functional properties of available antibody inhibitors to αvβ8 to gain insights into which antibody properties translated to efficacy, and then to leverage structural biologic insights to optimize the efficacy of humanized antibody inhibitors to αvβ8.

To test antibody inhibitors we used a cell-based system, which we developed to mimic the biologically relevant interactions between an αvβ8 expressing cell with a L-TGF-β presenting cell. In this system, the L-TGF-β presenting cell is also the cell that responds to TGF-β signaling, which is the case when a L-TGF-β presenting T-cell encounters an αvβ8 expressing tumor, stromal or immune cell in the tumor microenvironment (Seed, et al (2021) Sci. Immunol. 6, eabf0558). The most widely used in vitro TGF-β activation system relies on co-culturing a highly sensitive and specific TGF-β reporter cell line (TMLC) with integrin-expressing and/or L-TGF-β/GARP presenting cells (Wang, et al (2012) Mol Biol Cell (6): 1129-39). In such systems, TMLC must be in contact with these other cell-types for detection of TGF-β activation (Munger, et al (1999) Cell 96 (3): 319-28). TMLC cells are a stable subclone of mink lung epithelial cells (MLEC) stably transfected with an expression construct containing a TGF-β specific promoter fragment, consisting of a truncated plasminogen activator inhibitor-1 (PAI-1) promoter, fused to the firefly luciferase reporter gene (Abe, et al (1994) Anal Biochem 216 (2): 276-84). TMLC cells are widely used to measure active TGF-β because of their low background, specificity for TGF-β and abundant expression of TGF-β receptors and downstream signaling molecules that allow measurement of dose-dependent increases in TGF-β concentration in the physiologic range (Abe, et al (1994) Anal Biochem 216 (2): 276-84). However, TMLC cells cannot report cell-intrinsic TGF-β activation since they do not present L-TGF-β themselves.

To build a cell-intrinsic TGF-β activation system, TMLC cells were stably transfected with wild-type (WT) TGF-β. Without co-transfecting GARP, TMLC do not present L-TGF-β on their cell surface. When co-transfected with TGF-β (SEQ ID NO: 50) and GARP (SEQ ID NO: 54), high levels of cell surface expression of L-TGF-β can be detected (Campbell, et al (2020) Cell 180 (3): 490-501). These TMLC expressing L-TGF-β on their cell surface report cell-intrinsic TGF-β activation since when we co-transfected with a mutant non-releasable form of TGF-β (R249A) and GARP, robust αvβ8-mediated activation can be detected (Campbell, et al (2020) Cell 180 (3): 490-501). Since the R249A mutation prevents cleavage of TGF-β from its prodomain during biosynthesis, these experiments together with the absence of mature TGF-β present in the supernatant of TMLC expressing GARP/L-TGF-plated on immobilized αvβ8, demonstrate that any αvβ8-mediated activation in this system is only on the TGF-β/GARP expressing cell.

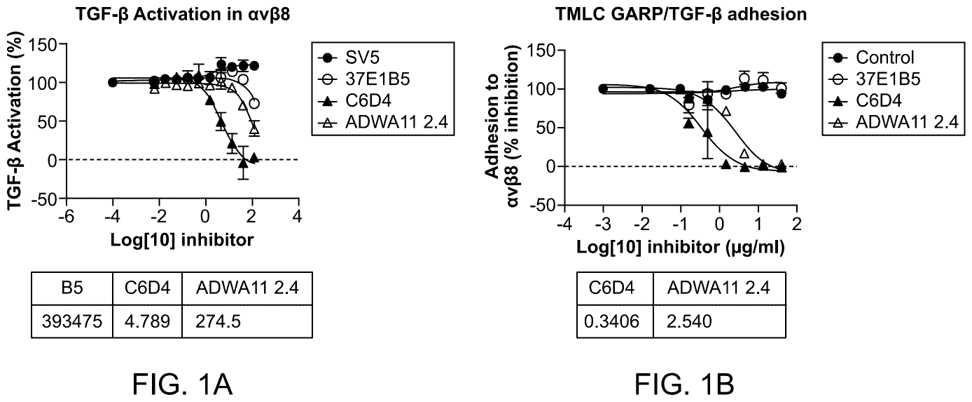

TMLC non-transfected or transfected with WT L-TGF-β, or WT L-TGF-β/GARP were plated on the immobilized αvβ8 ectodomain, or control substrate (BSA) (FIG. 1). The robust activation of TGF-β from the L-TGF-β/GARP complex on TMLC plated on αvβ8 could be blocked efficiently by C6D4 compared with ADWA11 2.4 or 37E1B5. The later is an allosteric inhibitor directed to an epitope distant from the ligand binding domain on the outer edge of the al-helix of the β8 subunit and is known to only partially block αvβ8-mediated TGF-β activation (Minagawa, et al (2014) Sci Trans Med, 6 (241).

### Example 2. Cryogenic Electron Microscopy (Cryo-EM) of ADWA 11 2.4 in Complex with Human αvβ8

The structure of recombinant Human Integrin alpha V beta 8 (αvβ8) protein with the ADWA 11 2.4 Fab was determined by cryo-EM to determine the epitope on αvβ8 that binds to ADWA 11 2.4, the paratope of ADWA 11 2.4, and how ADWA 11 2.4 inhibits αvβ8-mediated TGF-β activation.

### Sample and Grids Preparation.

ADWA 11 2.4 (SEQ ID NOs: 61 and 62) Fab was prepared by papain digestion and purified by Pierce Fab Preparation Kit. αvβ8 was generated as a recombinant protein by transfection of ExpiCHO cells with plasmids encoding the entire ectodomains of human αv (Seq ID NO: 52) and human β8 (Seq ID NO:53) and supplied in PBS buffer (10 mM sodium phosphate, 150 mM sodium chloride, 1 mM CaCl₂), 1 mM MgCl₂, pH 7.4). The sample used for cryo-EM experiments was prepared by mixing 8 μl of ADWA11 Fab (22 μM), 12 μl of αvβ8 (5 μM), and 20 μl of PBS buffer (10 mM sodium phosphate, 150 mM sodium chloride, pH 7.4) for a final concentration of 4.4 μM for Fab HuF12 and 1.5 μM for αvβ8.

Grids (Quantifoil, Au 300 mesh, R1.2/1.3) were prepared using a Vitrobot Mark 4 (ThermoFisher) using standard procedures. Grids were glow discharged using a Pelco easyGlow unit (Ted Pella, Inc.) with the factory suggested values for plasma cleaning (0.39 mbar, lower level 15 mA, hold 30″, glow 30″). The Vitrobot was set with a chamber humidity between 100%; a chamber temperature of 22° C.; a blot time of 1 sec; a wait time of 0 sec; a blot force of 1. 2.8 μl of sample were applied to the grid, blotted, and then plunged into a liquid ethane bath; the frozen grid was then transferred to liquid nitrogen (LN2) and kept at LN2 temperature for all subsequent steps (clipping, transferring to the microscope cassette, and data collection).

### Data Collection and Structure Determination.

The data set was collected on a ThermoFisher 200 KeV Talos Arctica equipped with a GATAN K3 direct detector camera. Data collection was done using SerialEM. 917 movies were collected at a nominal magnification of 45,000×; the defocus range was Set to be between-1.5 and -2.5 μm. The detector pixel size was 0.89 Å and the dose was 62 e⁻¹/Å².

### Data Processing and Map Reconstruction.

The entire data processing and map reconstruction was carried out with cryoSPARC V3. The initial particle picking identified 271,288 particles. After a round of 2D classification, following a round of Ab-Initio Reconstruction combined with Heterogeneous Refinement to clean the particles, about 46,961 particles were used to calculate an initial map (nominal resolution 7.41 Å, bin by 4). After bin back to 1, all particles were used to generate a map that after a NU-refinement had a nominal resolution of 4.11 Å. This map was used to build the model.

### Model Building and Refinement

All model building and refinement were carried out using PHENIX and COOT. avb8 in PDB: 6UJB and alpha-fold2 predicated ADWA11 Fab were used as the starting model. The COOT was used to fit some not-well-fitted regions. The PHENIX real space refinement module was carried out to optimize the model geometry. Table I summarizes the model refinement and statistics.

### Structural Analysis.

The structure was determined to overall resolution of 4.11 Å. The quality of the cryo-EM map at the binding interface was such that assignment of interacting side chains for both the antigen and the Fab was unequivocal.

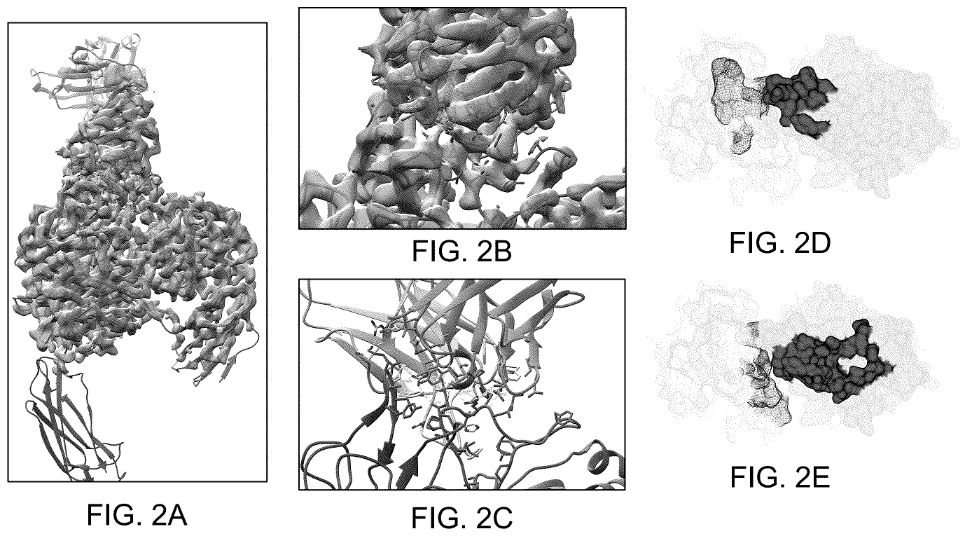

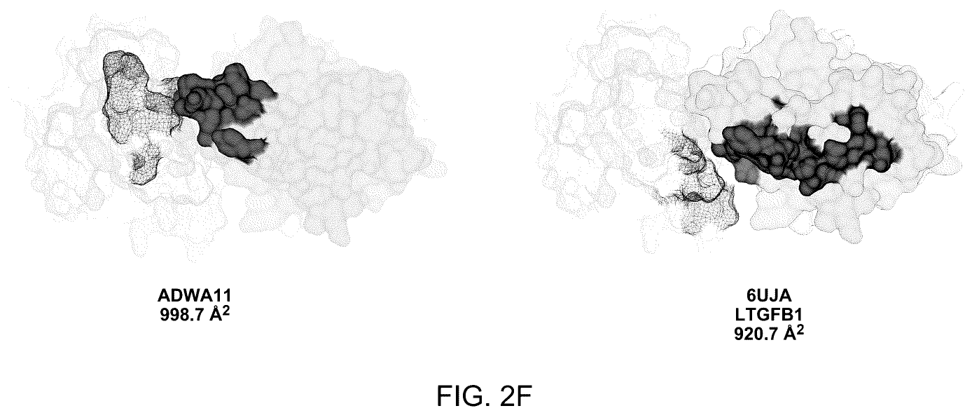

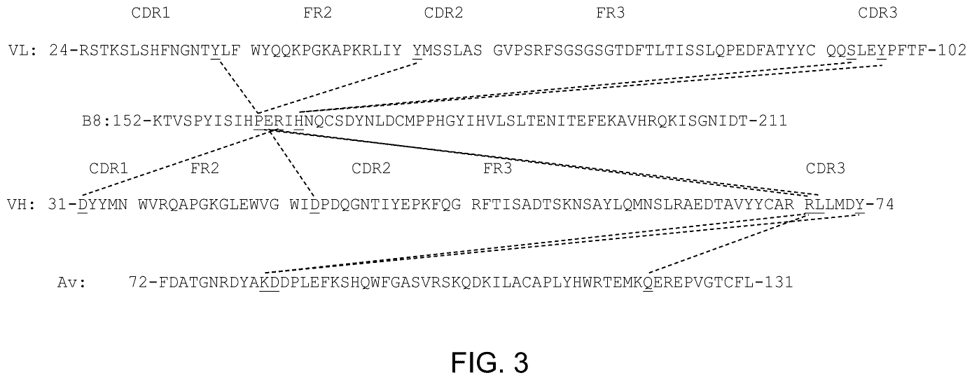

The ADWA 11 2.4-Fab paratope and αvβ8 epitope residues that comprise the interaction interface are shown in FIGS. 2 and 3. The interface is made up of van der Waals and electrostatic interactions, and corresponds to −998.7 Å² of buried surface, as calculated by PISA. The epitope is formed by residues Lys 82, Arg 83 and Gln 120 from the αv and Glu 163, Arg 164, Pro 162, and His 166 from the β8 chain. The HuF12 paratope is formed by residues Asp 31, Asp 52, Arg 99, Leu 100, and Tyr 104 from the heavy chain (V_{H}) and Tyr 37, Tyr 55, Ser 96 and Tyr 99 from the light chain (V_{L}) chain (FIG. 3). The ADWA 11 2.4 Fab epitope on the αv-subunit has 0% overlap with the αv-residues that interact with L-TGF-β1 (PDB: 6UJA) and 8.3% overlap with the β8-residues that interact with L-TGF-β1 (PDB: 6UJA). This is in contrast to the C6D4 Fab epitope which has 80% overlap with the αv-residues that interact with L-TGF-β1 (PDB: 6UJA) and 71% overlap with the β8-residues that interact with L-TGF-β1 (PDB: 6UJA). The fact that the ADWA-11 2.4 binding interface incompletely obstructs the binding interface of LAP of TGF-β1 (FIG. 2F, see PDB: 6UJA) compared to C6D4 (PDB: 6UJB) which more completely blocks the binding interface of αvβ8 with the LAP of TGF-β1 (FIG. 2E) explains why ADWA 11 2.4 relatively inefficiently blocks αvβ8-mediated TGF-β activation compared with C6D4 (FIG. 1).

### Example 3. Cryogenic Electron Microscopy (Cryo-EM) of HuF12 in Complex with Human αvβ8

The structure of recombinant Human Integrin alpha Vbeta 8 (αvβ8) protein with the HuF12 Fab was determined by cryo-EM to determine the epitope on αvβ8 that binds to HuF12 binds, the paratope of HuF12, and how HuF12 inhibits αvβ8-mediated TGF-β activation.

### Sample and Grids Preparation.

HuF12 (SEQ ID NOs: 24 and 25) Fab was prepared by papain digestion and purified by Pierce Fab Preparation Kit. αvβ8 was generated as a recombinant protein by transfection of ExpiCHO cells with plasmids encoding the entire ectodomains of human αv (Seq ID NO: 53) and human 38 (Seq ID NO: 52) and supplied in PBS buffer (10 mM sodium phosphate, 150 mM sodium chloride, 1 mM CaCl₂), 1 mM MgCl₂, pH 7.4). The sample used for cryo-EM experiments was prepared by mixing 4 μl of HuF12 Fab (48 μM), 12 μl of αvβ8 (5 μM), and 24 μl of PBS buffer (10 mM sodium phosphate, 150 mM sodium chloride, pH 7.4) for a final concentration of 4.8 UM for Fab HuF12 and 1.5 μM for αvβ8.

Grids (Quantifoil, Au 300 mesh, R1.2/1.3) were prepared using a Vitrobot Mark 4 (ThermoFisher) using standard procedures. Grids were glow discharged using a Pelco easyGlow unit (Ted Pella, Inc.) with the factory suggested values for plasma cleaning (0.39 mbar, lower level 15 mA, hold 30″, glow 30″). The Vitrobot was set with a chamber humidity between 100%; a chamber temperature of 22° C.; a blot time of 1 sec; a wait time of 0 sec; a blot force of 1. 2.8 μl of sample were applied to the grid, blotted, and then plunged into a liquid ethane bath; the frozen grid was then transferred to liquid nitrogen (LN2) and kept at LN2 temperature for all subsequent steps (clipping, transferring to the microscope cassette, and data collection).

### Data Collection and Structure Determination.

The data set was collected on a ThermoFisher 200 KeV Talos Arctica equipped with a GATAN K3 direct detector camera. Data collection was done using SerialEM. 1221 movies were collected at a nominal magnification of 45,000×; the defocus range was Set to be between −1.5 and −2.5 μm. The detector pixel size was 0.89 Å and the dose was 62 e⁻¹/Å².

### Data Processing and Map Reconstruction.

The entire data processing and map reconstruction was carried out with cryoSPARC V3. The initial particle picking identified 575,075 particles. After several rounds of 2D classifications, in the meantime together with Ab-Initio Reconstruction combined with Heterogeneous Refinement to get and save all the right particles, about 160,271 particles were used to calculate an initial map (nominal resolution 7.41 Å, bin by 4). After bin back to 1, all particles were used to generate a map that after a NU-refinement had a nominal resolution of 3.12 Å. This map was used to build the model.

### Model Building and Refinement

All model building and refinement were carried out using PHENIX and COOT. The complex between αvβ8 and C6D4 (PDB: 6UJB) was used as the starting model. The COOT was used to mutate sequence from C6D4 to HuF12 and fit some not-well-fitted regions. The PHENIX real space refinement module was carried out to optimize the model geometry. Table II summarizes the model refinement and statistics.

### Structural Analysis.

The structure was determined to overall resolution of 3.12 Å. The quality of the cryo-EM map at the binding interface was such that assignment of interacting side chains for both the antigen and the Fab was unequivocal.

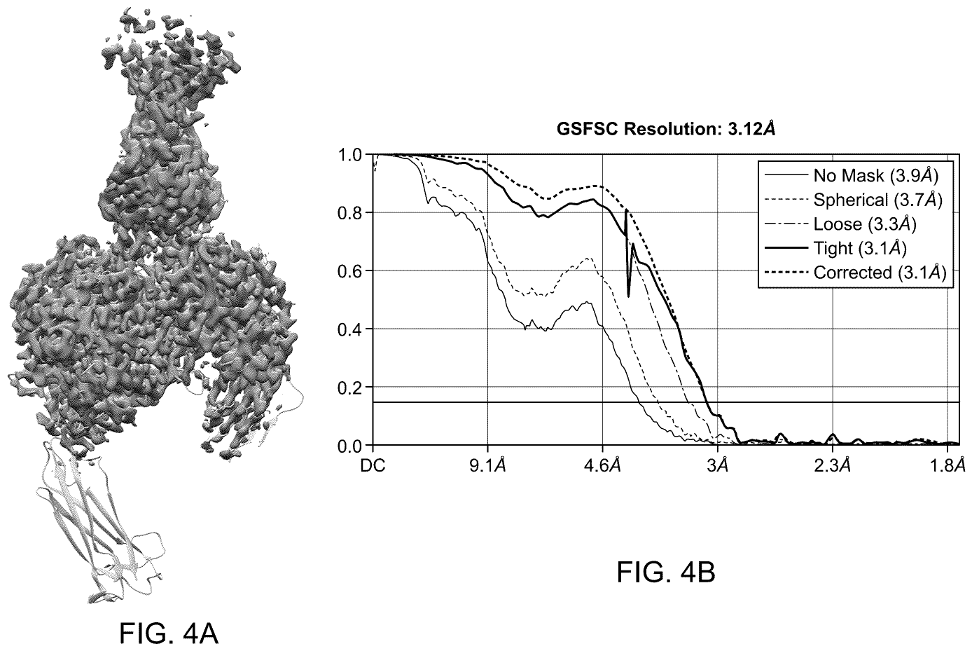

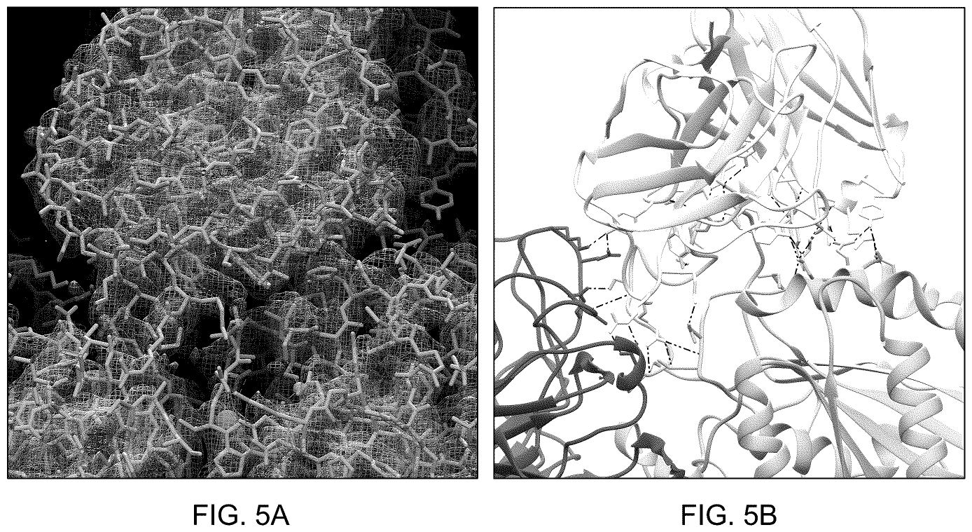

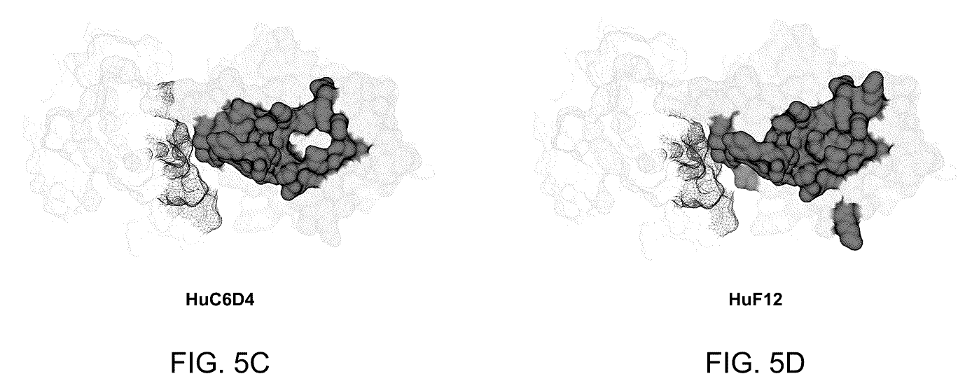

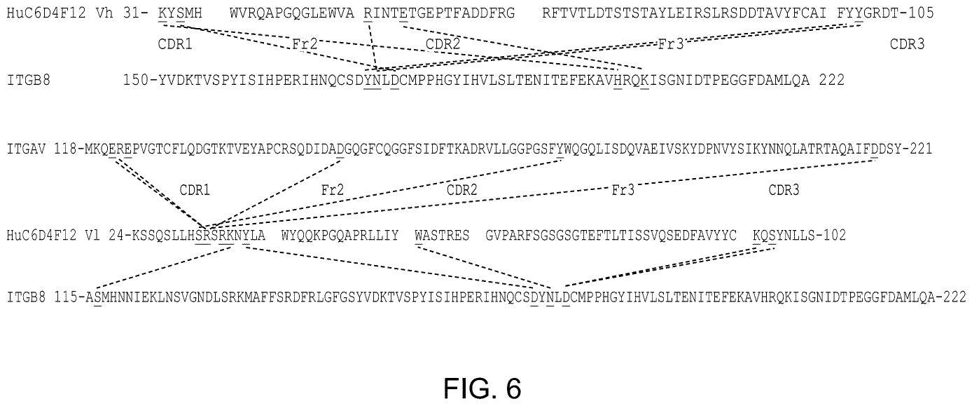

The humanized HuF12-Fab paratope and αvβ8 epitope residues that comprise the interaction interface are shown in FIGS. 4 and 5, and summarized in Table III. See also FIG. 6. The interface is made up of van der Waals and electrostatic interactions, and corresponds to 1420.2 Å² of buried surface, as calculated by PISA, which is larger than that of huC6D4 which corresponds to 1307 Å² of buried surface (FIG. 5). The epitope is formed by residues from both the αv and the β8 chain as summarized in Table II and shown in FIG. 6. The HuF12 paratope is formed by residues from both the VH and the VL chain as summarized in Table II and shown in FIG. 6. The fact that the huF12 binding interface almost completely obstructs the binding interface of LAP of TGF-β1 residues explains why the antibody efficiently blocks αvβ8-mediated TGF-β activation. The HuF12 epitope on both the αv and β8-subunits is 100% conserved between mammalian species (including non-human primate, mouse, rat, sheep, cow, horse, dog, among others) explaining why HuF12 is broadly species cross-reactive. Key interacting residues (av: Glu 123, Asp 150; β8: Asp 171, Tyr 172, Asn 172, Asp 175, His 200) are not conserved between other integrin α and β subunits explaining the structural basis of the strict specificity of HuF12 for αvβ8.

### Example 4. In Vivo Function of Anti-αvβ8 Antibodies is Influenced by Epitope Selection and Antibody Affinity

Comparisons of structures of ADWA-11 2.4 and C6D4 indicated that they bind to overlapping but partially overlapping epitopes. ADWA-11 2.4 bound to the αv-integrin head domain outside of the ligand binding pocket and to a portion of the β8 subunit, which resulted in partial occlusion of the ligand binding pocket (FIG. 2). The C6D4 antibody more completely covers the ligand binding pocket, explaining the relative improvement in ability to inhibit αvβ8-mediated activation of cell-surface GARP/L-TGF-β (FIG. 1). The allosteric inhibitor, B5 (Minagawa, et al, Sci Trans Med. 2014 Jun. 18; 6 (241): 241ra79), like ADWA-11 2.4, only partially inhibited αvβ8-mediated activation of cell-surface GARP/L-TGF-β (FIG. 1). This result shows the importance of epitope selection in inhibiting αvβ8-mediated activation of cell-surface GARP/L-TGF-β and shows that more complete coverage of the αvβ8 ligand binding pocket is a desirable feature for antibody efficacy in inhibition of αvβ8-mediated TGF-β activation.

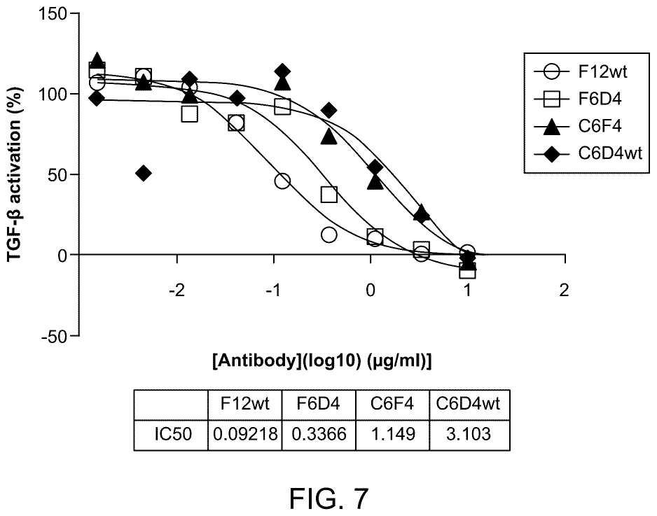

We have made a version of C6D4 that differs in a single amino acid in both the VH and VL domains. The VH mutation replaces the Asp (SEQ ID NO: 22) with a Arg in the first position of CDR1 Vh (SEQ ID NO: 24), and the VL mutation replaces a Asn (SEQ ID NO: 23) with a His in the middle of CDR1 VL (SEQ ID NO 25). HuF12 has an affinity as measure by KinExA of 2.36 pM (95% CI: 4.06 pM-1.04 pM), compared to HuC6D4: 82.09 pM (95% CI: 95.74 pM-69.60 pM). The dramatic increase in affinity of huF12 compared to HuC6D4 is mainly due to a single K31R mutation in the CDR1 of the HuF12 VH. Thus, when the respective composite antibodies are made (i.e. swapping the VH and VL), the combination of huF12 VH and huF12 VL is more effective than huF12 VH with huC6D4 VL, which is more effective than huC6D4 VH with huF12 VL, which is more effective than HuC6D4 VH and HuC6D4 VL (FIG. 7). These results clearly indicate that the D31R mutation of CDR1 VH is a useful mutation in changing the affinity of huF12 for αvβ8.

### Example 5. In Vivo Function of Anti-αvβ8 Antibodies is Influenced by Epitope Selection and Antibody Affinity

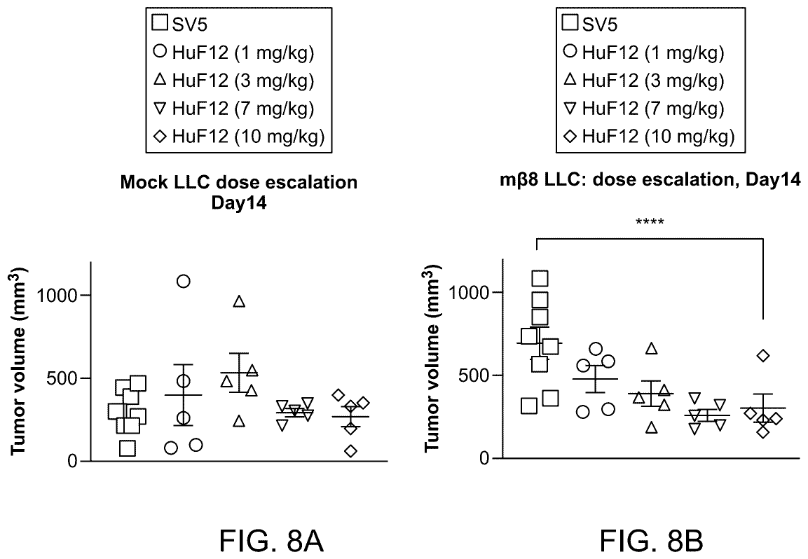

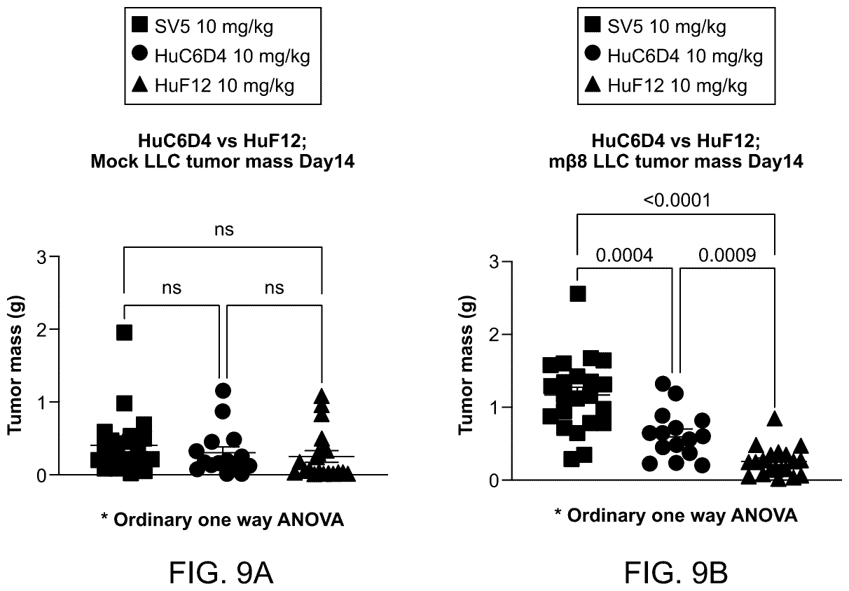

As an example of the importance of affinity in the anti-tumor function of αvβ8-domain antibodies, we made side-by-side comparisons of the ability of huC6D4 and huF12 to inhibit growth in mice of Lewis Lung Carcinoma (LLC) cells either transfect to express β8 or mock transfected. The transfected cells were selected and sorted for high uniform expression of β8 (β8 LLC) (Takasaka, et al (2018) JCI Insight (3; 20). Day 4 after tumor cell injection, tumors were palpable and the mice were randomized and injected with either SV5 (isotype IgG2 control), or escalating doses of HuF12-IgG2a (1-10 mg/kg, IP). Injections were repeated again on day 11, and tumors harvested on day 14. HuF12 showed a statistically significant trend for dose-response (FIG. 8). We therefore repeated the experiment using the highest dose tested, and injected mice with palpable β8 LLC or mock LLC tumors with either SV5 (isotype IgG2 control), HuC6D4-IgG2a, or HuF12-IgG2a (10 mg/kg, IP). HuF12 was significantly better than HuC6D4 at inhibiting β8 LLC tumor growth indicating that improved affinity also improves the ability of an antibody to the HuC6D4 epitope to inhibit tumor growth (FIG. 9).

### Example 6. Epitope Selection is Important for Anti-Tumor Efficacy of Anti-β8 Antibodies

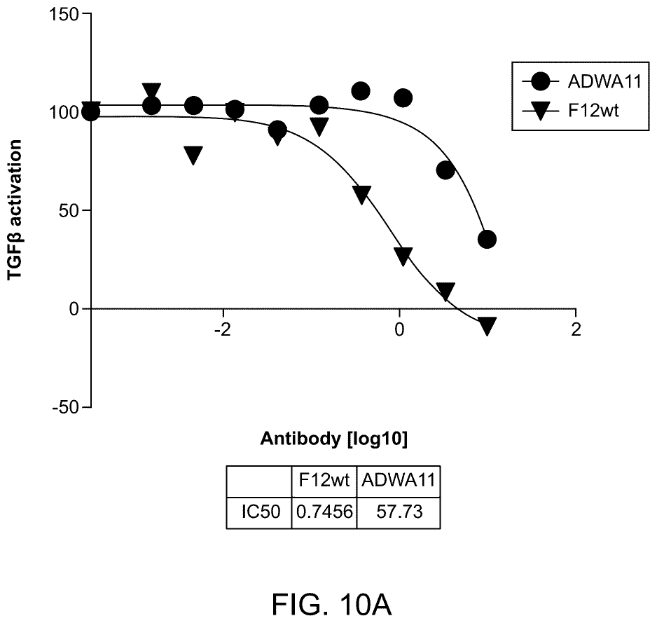

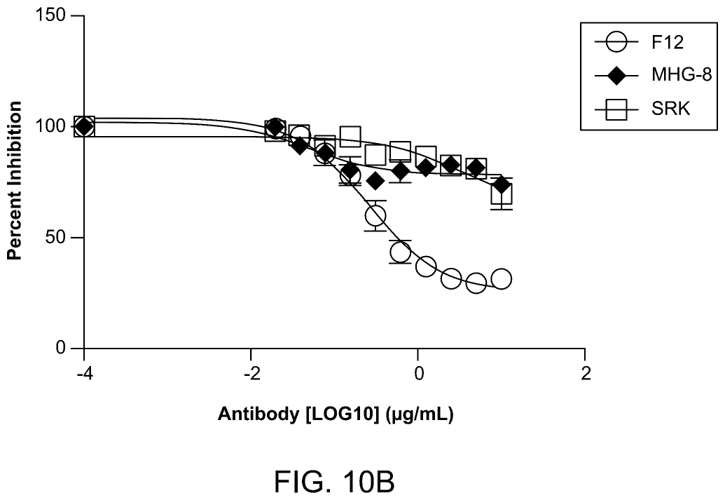

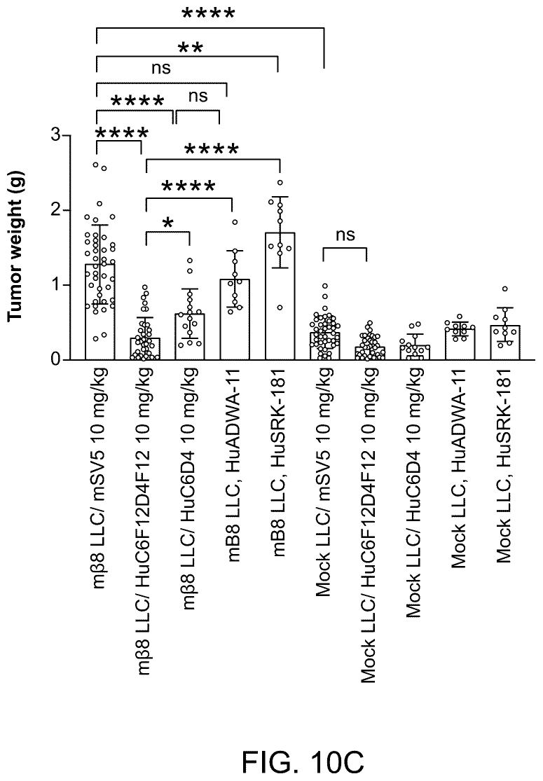

ADWA 11 2.4 binds to the edge of the αvβ8 ligand binding pocket while HuC6D4 and HuF12 almost completely covers the αvβ8 ligand binding pocket (FIG. 5). HuF12 blocks αvβ8-mediated TGF-β activation better than HuC6D4 (FIG. 7) and much better than ADWA 11 2.4 (FIG. 10). A number of studies suggest that the TGF-β1 itself can be targeted or that its activation can be inhibited by stabilizing the latent domain (LAP) of TGF-β1 (Cuende, et al (2015) Sci Transl Med, 7 (284); Martin, et al (2020) Sci Transl Med, 12 (536)). We have tested a pan-TGF-β inhibitor 1D11, anti-TGF-βR2 antibodies, anti-GARP/TGF-β (MHG-8) antibodies and TGF-βR2-Fc protein to block αvβ8-mediated activation of TGF-β. None of these antibodies blocked αvβ8-mediated TGF-β activation as well as HuC6D4 (Seed, et al (2021) Sci Immunol 6, eabf0558). We directly compared the ability HuF12 to block αvβ8-mediated activation of TGF-β compared to MHG-8 and to another anti-LAP antibodies, SRK-181 (Ab6) (SEQ ID Nos: 64, 65), which is cross-reactive against mouse and human L-TGF-β1 (Martin, et al (2020) Sci Transl Med, 12 (536). HuF12 potently inhibited αvβ8-mediated activation of TGF-β while MHG-8 and SRK-181 (Ab6) blocked poorly (FIG. 10). We performed side-by-side (all at 10 mg/kg) in vivo anti-tumor efficacy tests against β8 LLC tumors using HuC6D4, HuF12, ADWA-11 2.4 and SRK-181 (Ab6). HuF12 significantly inhibited 38 LLC tumor growth greater than HuC6D4 and ADWA 11 2.4, while SRK-181 (Ab6) has no significant growth inhibition relative to control (FIG. 10). These data demonstrate that the increased affinity of HuF12 compared to HuC6D4, and epitope selection are useful for both in vitro function and anti-tumor activity in vivo.

### Example 7. Further Humanization of HuF12 Using a Structure Based Approach

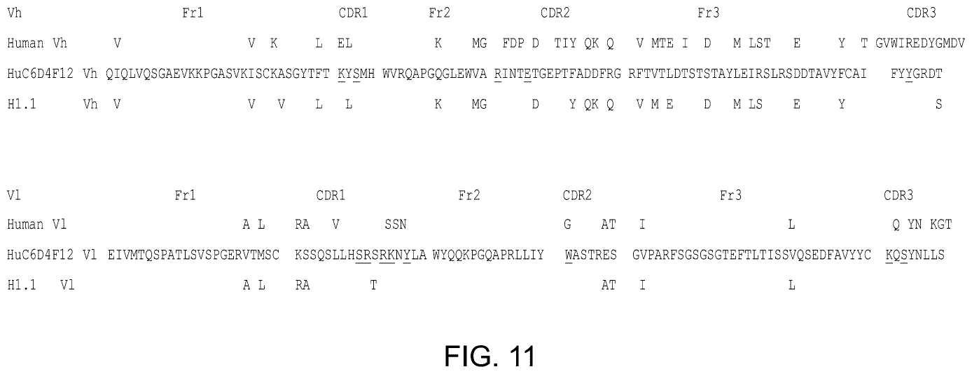

We have shown that both affinity and epitope selection for anti-β8 antibodies are important for anti-tumor activity in vivo. However, HuF12 contains numerous murine amino acids in its frameworks, and can therefore potentially generate immunogenic responses in humans. The goal of antibody humanization is to produce antibody therapeutics that do not elicit an immune response, maintain their efficacy and are safe for use in humans. Thus, we sought to create a more humanized variant of HuF12 that maintained its affinity and selectivity for the HuC6D4 epitope. Starting with the HuF12 structure, we sought to substitute human residues into positions occupied by murine residues where the side-chains were exposed to solvent and therefore more likely to be immunogenic (i.e. surface veneering). Mouse residues that were buried, formed extensive interactions within beta sheets, or interacted with positioning of CDRs, were maintained. Additionally, where possible, we combined our structural approach with more standard framework-homology-based humanization, CDR-homology-based humanization, and specificity determining residues (SDR) grafting. The general work-flow for humanization is shown in Table IV. Our approach resulted in clone H1.1 (SEQ ID NO: 70, 71, FIG. 11), which when expressed as human IgG4 (SEQ ID NO: 59) had approximately 70-fold lower affinity than HuF12 as measure using surface binding to CHO αvβ8 cells.

### Example 8. Affinity Optimization of Humanized Variants Using Directed Evolution

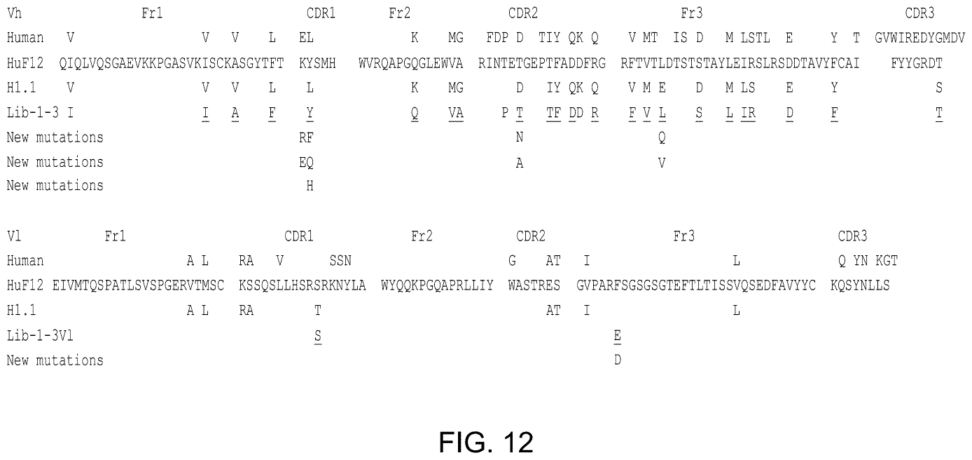

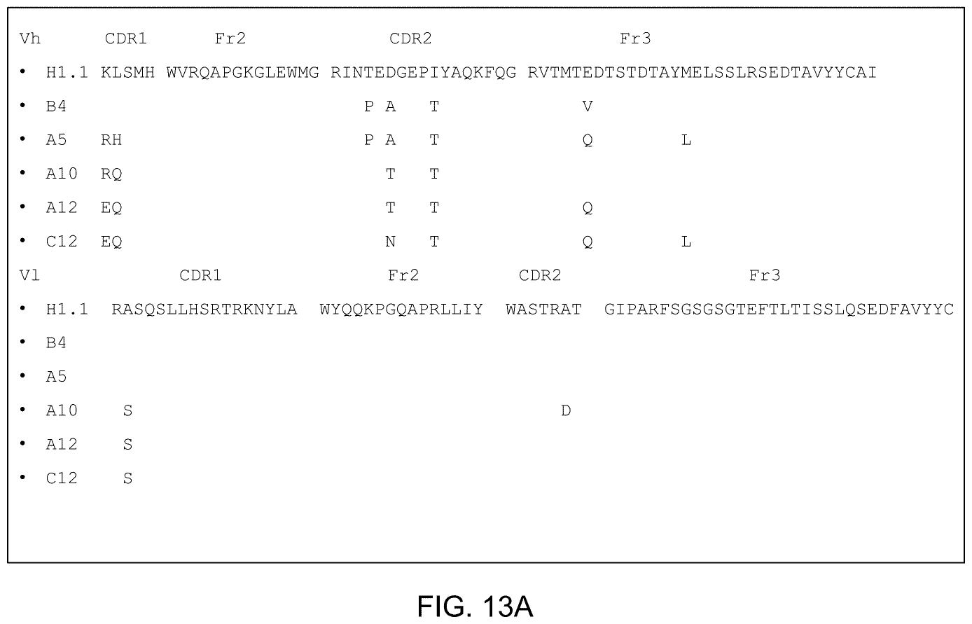

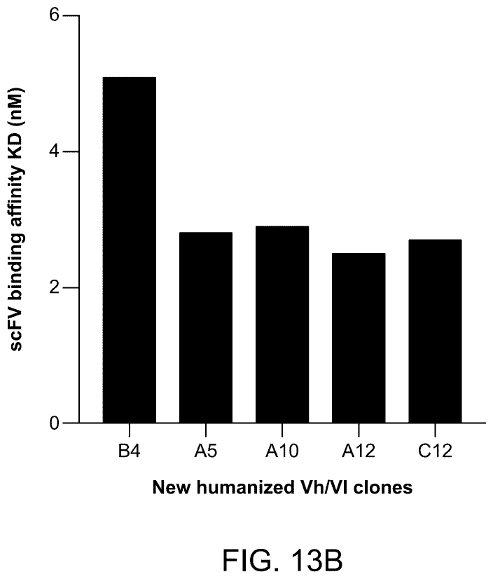

Antibody affinity during the humanization process can be improved typically through re-introducing the original murine amino acids into humanized positions (i.e. back mutations). Although, it is also possible to introduce de novo mutations in the frameworks and/or CDRs. As an example of such an approach, we built a library using Clone H1.1 as a template using the structure of HuF12/αvβ8 to select the amino acids in humanized positions to back-mutate, or to introduce de novo mutations. We chose to introduce de novo mutations in frameworks if those residues were buried and their side-chains not surface exposed, minimizing the potential immunogenicity of the de novo mutations. We also introduced de novo mutations in CDRs whose interactions were determined to be possible to optimize because they either did not interact or formed electrostatic interactions that were sub-optimal. The resulting library design was executed using degenerate codons in oligonucleotides which were tiled and amplified using splice-overlap extension PCR (FIG. 12). Degenerate positions in three libraries created sequentially coded for human (Vh: IGHV1-24_IGHD5-18_IGHJ6 (SEQ ID NO29; VL: IGKV3-15_IGKJ1 (SEQ ID NO: 33) or mouse residues. The scFV yeast display libraries were expressed in yeast using the scFV vector pYD4, which were transformed using gap-repair, as described (Takasaka, et al (2018) JCI Insight (3; 20)). After libraries were sorted for scFV expression and binding to high nM concentrations of the αvβ8 ectodomain, the resulting yeast pools were sequentially sorted with decreasing concentrations of αvβ8 ectodomain with increasingly stringent binding (Kon) and washing (Koff) steps. After three rounds of sorting, individual clones were isolated and sequenced, and stained individually using the αvβ8 ectodomain to measure binding affinity as scFV. Five prototype scFV were isolated using this process B4 (SEQ ID NOs: 68, 69), A5 (SEQ ID NOs: 72, 69), A10 (SEQ ID NOs: 73, 74), A12 (SEQ ID NOs: 75, 76), C12 (SEQ ID NOs: 77, 78) all which bound to αvβ8 with measurable affinity improved over Clone H1.1 (whose affinity was not measurable as scFV) (FIG. 13).

### Example 9. Identification and Characterization of Humanized Clone H4C8

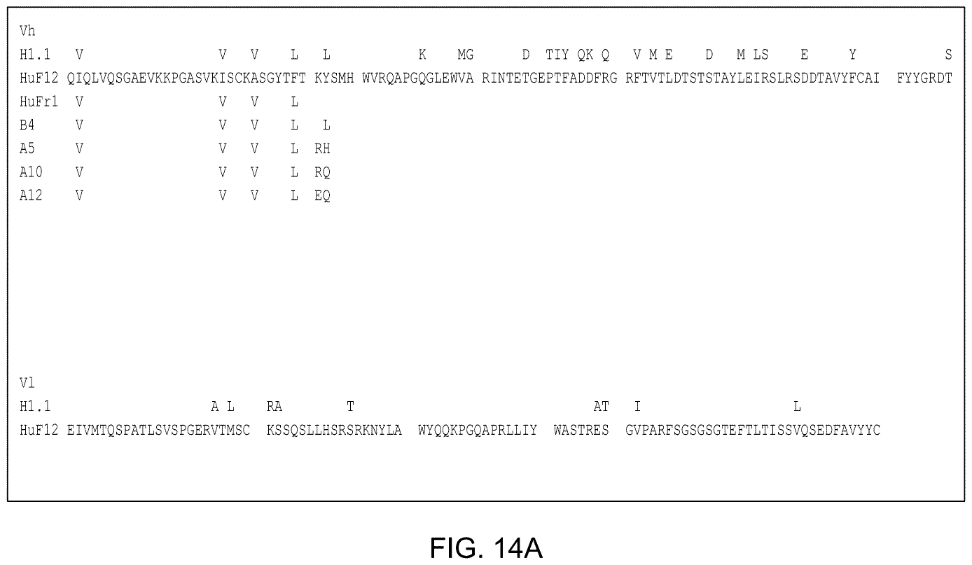

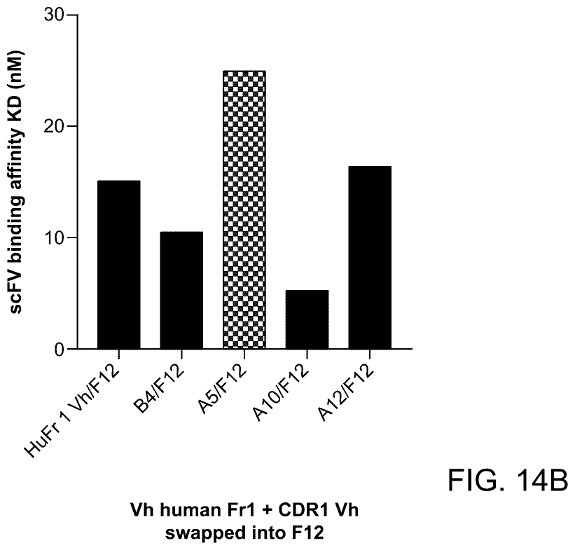

As an example of an approach to determine affinity liabilities of humanizing HuF12 we generated a series of chimeric scFV where various regions of clones B4, A5, A10 and A12 were swapped into HuF12 VH and VL domains to create chimeras (FIG. 14). Clones with a humanized Fr1 with either HuF12 Vh CDR-1, or the Vh CDR-1 from clones B4, A5, A10 or A12 were created by PCR (SEQ ID NOs 79-83), paired with the huF12 VL (SEQ ID NO: 25), and transformed into yeast into pYD4 using gap-repair, and sequences from individual clones verified, as described (see Takasaka, et al. JCI Insight (2018) 3; 20). After induction the transformants were assessed for scFV expression using anti-V5 (clone SV5). The αvβ8 ectodomain was incubated with the transformed yeast clones, washed and binding detected using anti-human αv (clone 8β8). Presence of the various amino acids coded for by degenerate codons were verified in sequences of individual clones. Individual clones were stained using the αvβ8 ectodomain to measure binding affinity as scFV. The result of this experiment revealed that when the HuF12 Fr1 was humanized, the CDR1 VH from clone A10 had the highest binding affinity as a scFV (FIG. 14).

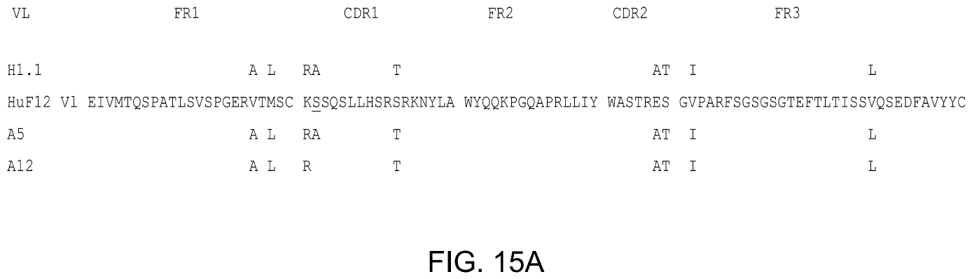

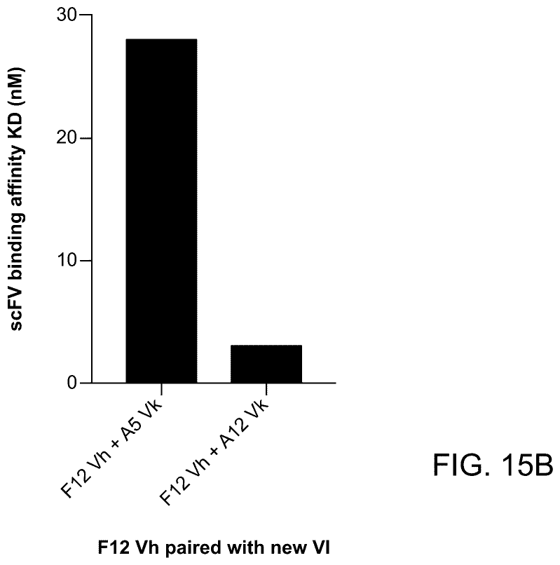

We then identified which humanized VL variants preserve binding when paired with HuC6D4F12 Vh. The VH from wild-type HuC6D4F12 (SEQ ID NO:24) was paired with the V_{L} from clones A5 (SEQ ID NO:71) and A12 (SEQ ID NO: 76), ligated in frame into the scFV vector pYD4 and transformed into yeast using gap-repair, as described (see Takasaka, et al. JCI Insight (2018) 3; 20). scFV expressing clones were stained using anti-V5 (clone SV5) and the αvβ8 ectodomain detected using anti-human αv (clone 8β8) (FIG. 15). The binding affinities were determined and the mouse residue (S25) in CDR1 was found to be the key mouse residue present in VL A5 but not VL A12 required for maintenance of affinity (FIG. 15).

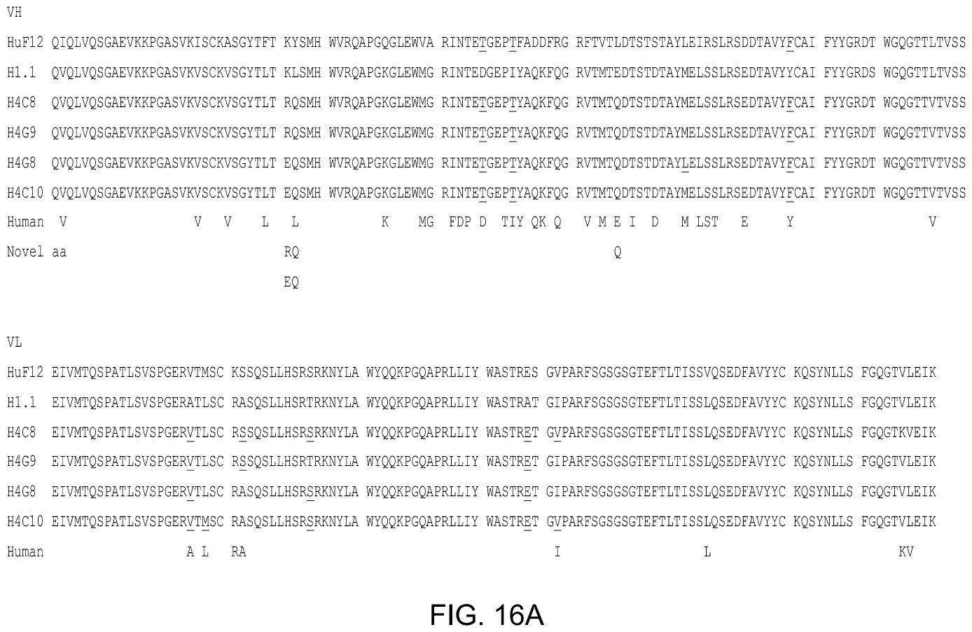

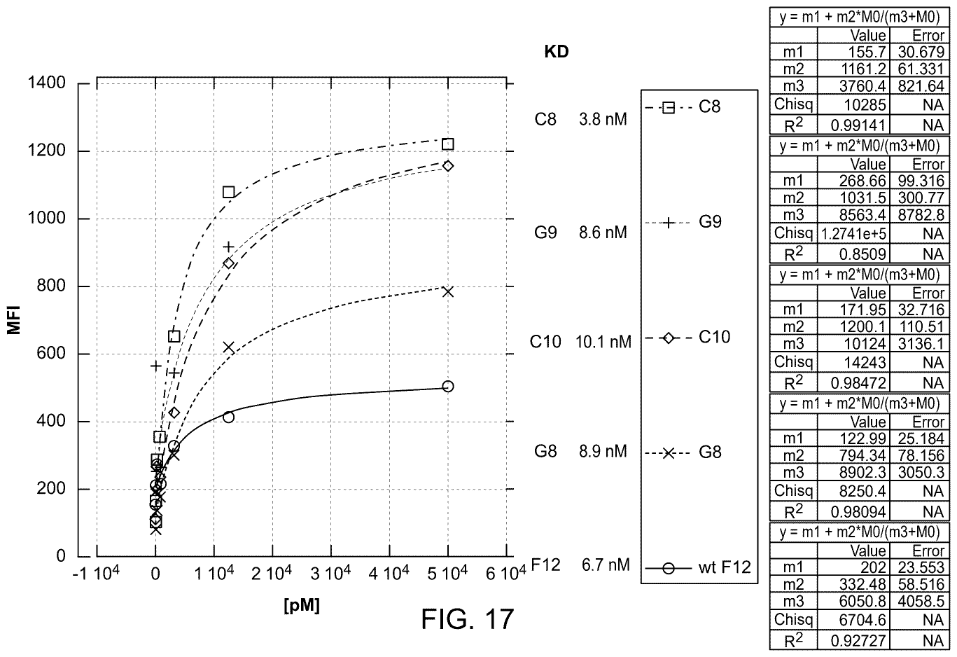

We then created a scFV yeast display library (Library 3) with degenerate positions coding for novel amino acids (i.e. not in germline human or mouse Ig sequence databases), back mutations to mouse from clone H1.1, as well as final humanization of Fr4 VH and VL based on the human germline (SEQ ID NO: 29, 30 and 32). Libraries were ligated in frame into the scFV vector pYD4 and transformed into yeast using gap-repair, as described (see Takasaka, et al. JCI Insight (2018) 3; 20). After induction and screening of the pooled transformants for scFV expression using anti-V5 (clone SV5), the αvβ8 ectodomain was incubated with the yeast pool, washed and scFV which bound to αvβ8 were detected using anti-human αv (clone 8β8). After multiple rounds of selection by sorting yeast stained under conditions of increasing stringency (Low Ag concentration, short Kon and long Koff), individual clones were isolated, sequenced and tested for binding affinity. Shown in FIG. 16 are the best binding clones (Clones H4C8: SEQ ID NO: 34, 35; H4G9: SEQ ID NO: 34, SEQ ID NO: 84; H4G8: SEQ ID NO: 85, SEQ ID NO: 86; H4C10: SEQ ID NO: 38, 87) enriched in the library. The binding affinities as scFV were determined to be huF12: 6.7 nM; H4C8: 3.8 nM; H4G9: 8.6 nM; H4G8: 8.9 nM; H4C10: 10.1 nM. The affinity of the H1.1 scFV was too low to be measurable (FIG. 17).

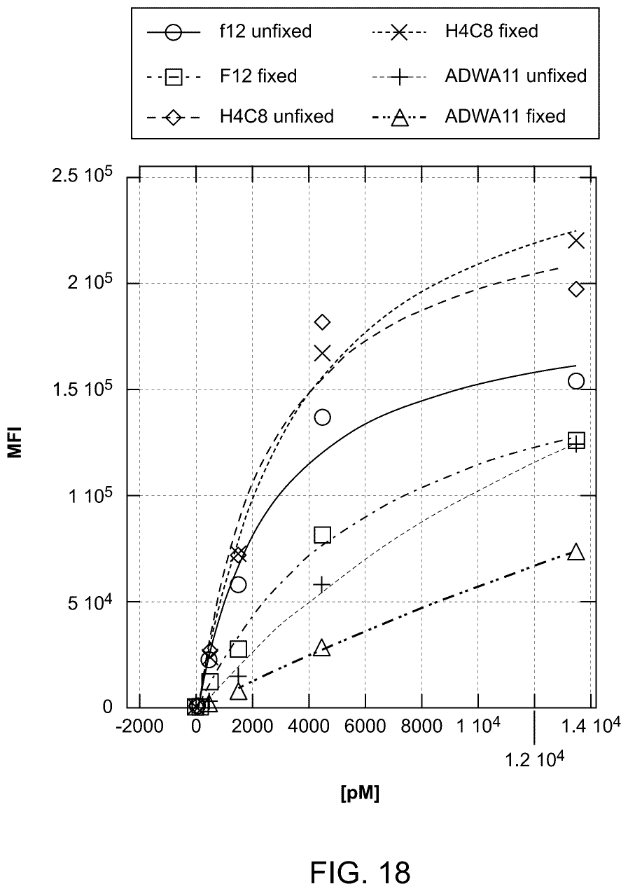

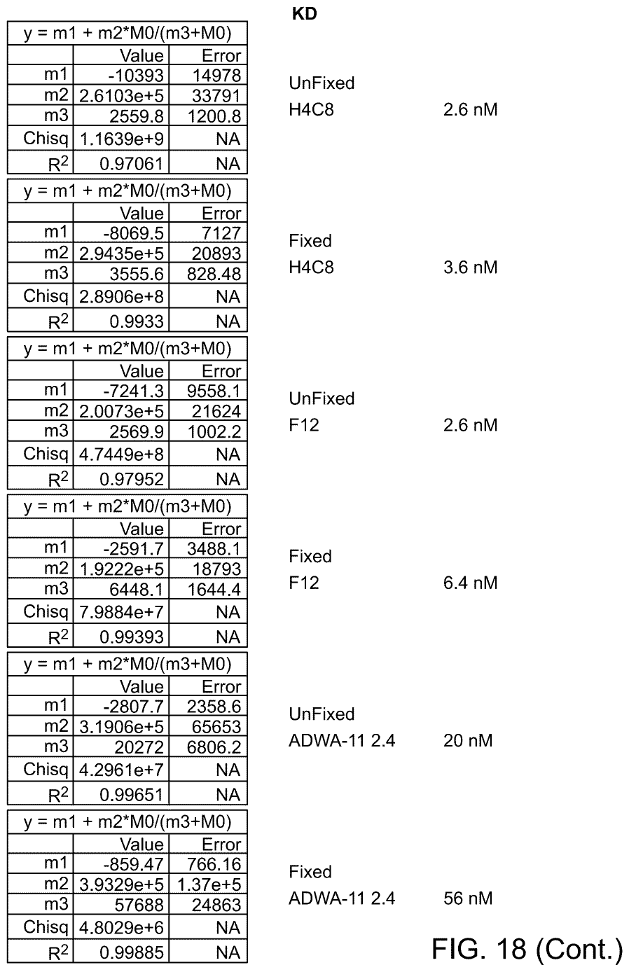

As an example of the change in epitope and antibody properties we tested H4C8 for its ability to bind to formalin fixed cells. The ability of an antibody to formalin fixed antigens is a desirable property in an antibody as it enables the staining of formalin fixed samples. H4C8 bound as well to fresh or formalin fixed αvβ8 expressing CHO cells, compared to HuC6D4F12 or ADWA11 2.4 (from Pat no. US2020/0079855 A1), which both showed decreased in binding affinity to formalin fixed cells (FIG. 18).

In antibody humanization, a number of computational models predict either the agreement with germline human immunoglobulin sequences or provide “humanness scores” based both on homology with germline human immunoglobulin sequences and comparison to other humanized and human therapeutic antibodies (Prihoda, et al (2022) MABS 14(1)). In one example when such an algorithm is applied to HuF12 (SEQ ID NO:24, 25) and H4C8 (SEQ ID NOs: 34, 35) it is found that HuF12 is in the 13^{th} and H4C8 is in the 48^{th} percentile. HuF12 has an identity score of 0.660, while H4C8 has an identity score of 0.778 of humanized/human antibodies in the database. Among antibodies in the database, this places HuF12 above the median of chimeric antibodies, but in the lower thresholds for humanized antibodies, while H4C8 is at or above the median for humanized antibodies (Table V).

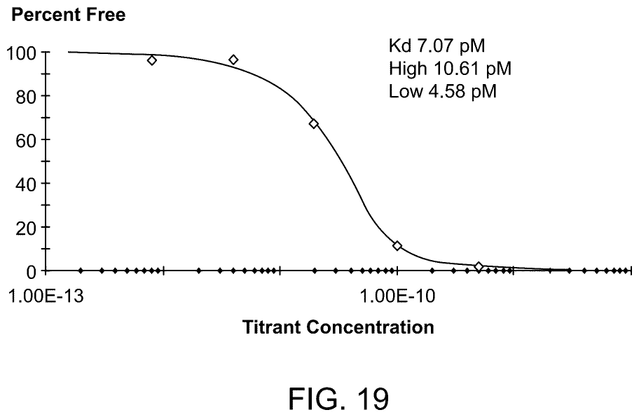

A Kinetic Exclusion Assay (KinExA®, Sapidyne Instruments, Inc., Boise, ID) was used to measure the binding affinity of H4C8 (Seq ID NOs. 34, 35) produced with the CH1-CH3 domain from IgG4 (Seq ID NO: 59). The solution-based affinity of H4C8 is 7.07 pM (range 4.58 to 10.61 pM) as measured by KinExA, which is a flow fluorimeter which allows true measurement of binding affinity and kinetics for unmodified molecules in solution, including antibody-antigen interactions (FIG. 19). Samples with serial dilutions are drawn over solid-phase beads coupled with one binding partner to capture the complimentary binding molecule, which is detected by flow of fluorophore-labeled antibody solution binding to a separate epitope on the same molecule being measured. Signals are used to calculate free concentration in solution after equilibrium is reached (i.e. to determine binding affinity KD), or under pre-equilibrium conditions over time (i.e. to determine binding kinetics). Free antigen remaining in solution at equilibrium for varying initial antibody concentrations in constant initial concentration of antigen as determined by the KinExA software, based on optimized least-squares fitting to a reversible binding equation.

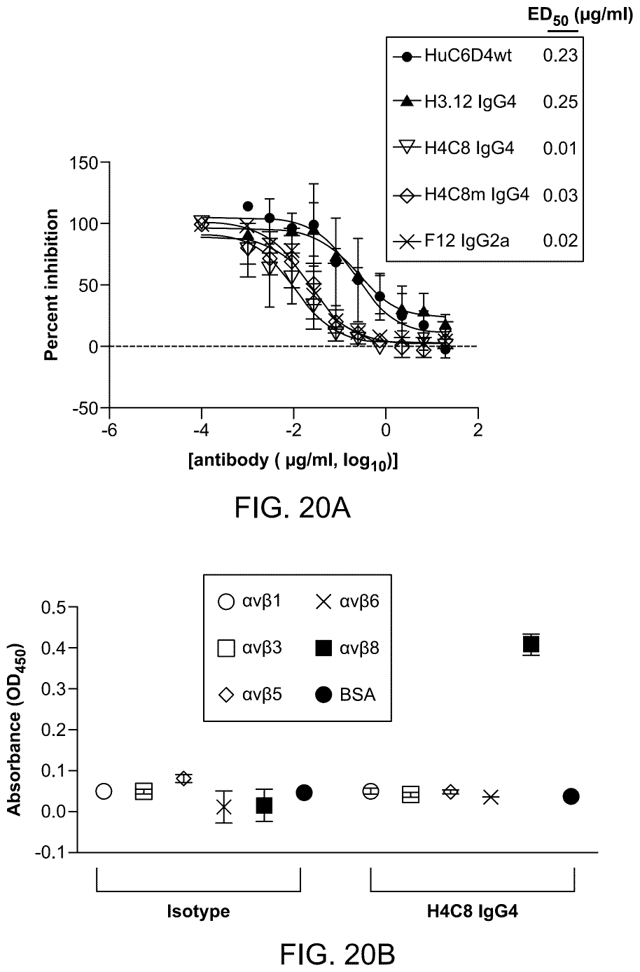

As an example to show that H4C8 maintains ability to block αvβ8-mediated TGF-β activation similar to HuC6D4F12 while maintaining specificity for αvβ8, we produced inhibition curves for H4C8 (with either a humanized (SEQ ID NO: 35), or non-humanized (H4C8m) Fr4 light chain (SEQ ID NO: 36)), HuF12 (IgG2a, SEQ ID NO: 24, 25), HuC6D4 (IgG2a, SEQ ID NO: 22, 23) or H3.C12 (SEQ ID NO: 66, 67) (FIG. 20). Using immobilized αvβ8 ectodomain with TMLC GARP/TGF-β1 reporter cells the antibodies had the following EC50 values: HuC6D4: 0.23 μM; HuF12: 0.2 μM; H4C8: 0.1 μM; H4C8m: 0.3 μM; and H3.C12: 0.25 μM. H4C8 maintains its specificity for αvβ8, as it show no binding to other αv-integrins (FIG. 20). This example provides evidence that H4C8 maintains or improves the in vitro activity of HuF12, that humanization of light chain Fr4 maintains or improves function, and that the R31E mutation in CDR1 V_{H} of H3.C12 maintains blocking function but reduces efficacy.

### Example 10. Cryogenic Electron Microscopy (Cryo-EM) of H4C8 in Complex with Human αvβ8

The structure of recombinant Human Integrin alpha Vbeta 8 (αvβ8) protein with the H4C8 Fab was determined by cryo-EM to determine the epitope on αvβ8 that binds to H4C8 binds, the paratope of H4C8, and how H4C8 inhibits αvβ8-mediated TGF-β activation.

### Sample and Grids Preparation.

H4C8 (SEQ ID NOs: 34 and 35) Fab was prepared by papain digestion and purified by Pierce Fab Preparation Kit. αvβ8 was generated as a recombinant protein by transfection of ExpiCHO cells with plasmids encoding the entire ectodomains of human αv (Seq ID NO: 53) and human 38 (Seq ID NO: 52) and supplied in PBS buffer (10 mM sodium phosphate, 150 mM sodium chloride, 1 mM CaCl₂), 1 mM MgCl₂, pH 7.4). The sample used for cryo-EM experiments was prepared by mixing 3.7 μl of H4C8 Fab (22 μM), 3.5 μl of αvβ8 (11.5 μM), and 32.8 μl of PBS buffer (10 mM sodium phosphate, 150 mM sodium chloride, pH 7.4) for a final concentration of 2.0 μM for Fab H4C8 and 1.0 μM for αvβ8.

Grids (Quantifoil, Au 300 mesh, R1.2/1.3) were prepared using a Vitrobot Mark 4 (ThermoFisher) using standard procedures. Grids were glow discharged using a Pelco easyGlow unit (Ted Pella, Inc.) with the factory suggested values for plasma cleaning (0.39 mbar, lower level 15 mA, hold 30″, glow 30″). The Vitrobot was set with a chamber humidity between 100%; a chamber temperature of 22° C.; a blot time of 1 sec; a wait time of 0 sec; a blot force of 1. 2.8 μl of sample were applied to the grid, blotted, and then plunged into a liquid ethane bath; the frozen grid was then transferred to liquid nitrogen (LN2) and kept at LN2 temperature for all subsequent steps (clipping, transferring to the microscope cassette, and data collection).

### Data Collection and Structure Determination.

The data set was collected on a ThermoFisher 300 KeV Titan Krios G2 equipped with a GATAN K3 direct detector camera. Data collection was done using SerialEM. 1384 movies were collected at a nominal magnification of 105,000×; the defocus range was Set to be between −1.5 and −2.5 μm. The detector pixel size was 0.834 Å and the dose was 68 e⁻¹/Å².

### Data Processing and Map Reconstruction.

The entire data processing and map reconstruction was carried out with cryoSPARC V3. The initial particle picking identified 375,761 particles. After several rounds of 2D classifications, in the meantime together with Ab-Initio Reconstruction combined with Heterogeneous Refinement to get and save all the right particles, about 106,895 particles were used to do NU-refinement, and had a nominal resolution of 3.19 Å. This map was used to build the model.

### Model Building and Refinement

All model building and refinement were carried out using PHENIX and COOT. The complex between avb8 and C6D4 (PDB: 6UJB) was used as the starting model. The COOT was used to mutate sequence from C6D4 to H4C8, and to adjust some less well-fit regions. The PHENIX real space refinement module was carried out to optimize the model geometry. Table VI summarizes the model refinement and statistics.

### Structural Analysis.

The structure was determined to overall resolution of 3.19 Å. The quality of the cryo-EM map at the binding interface was such that assignment of interacting side chains for both the antigen and the Fab was unequivocal.

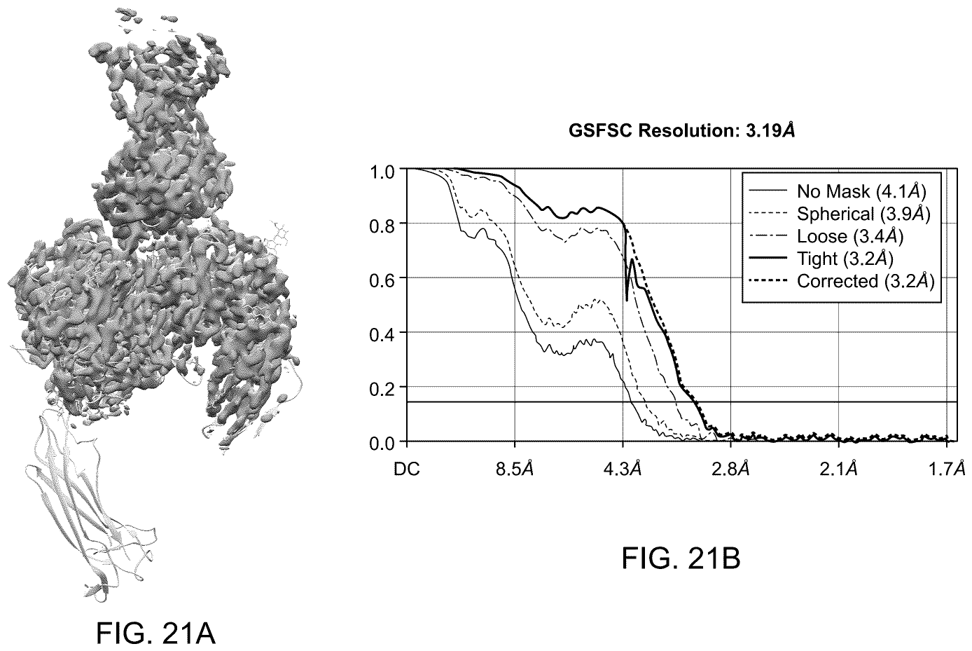

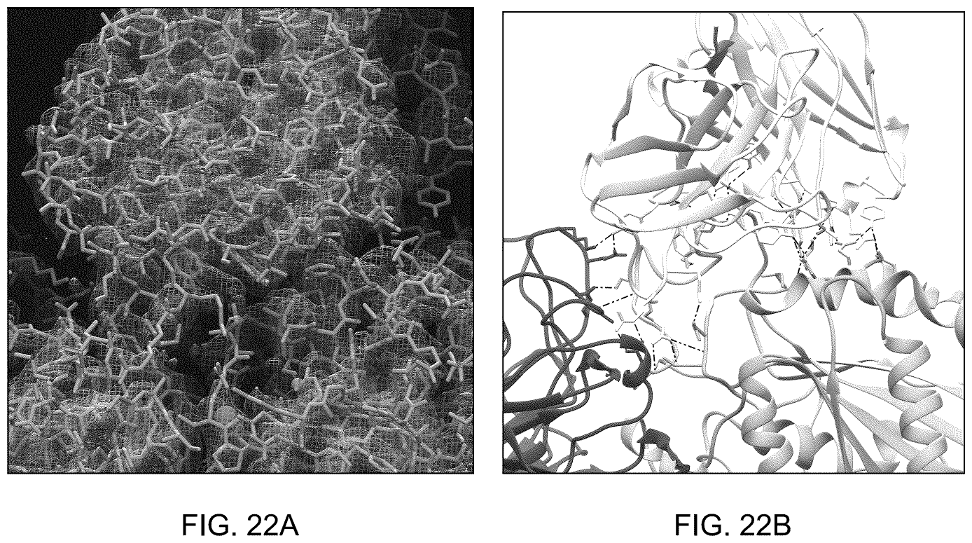

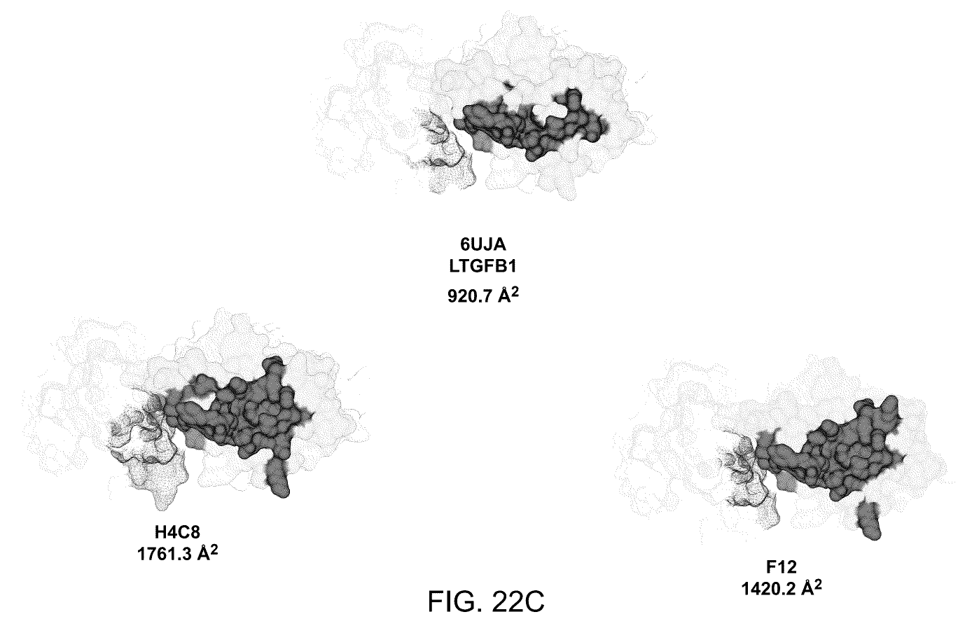

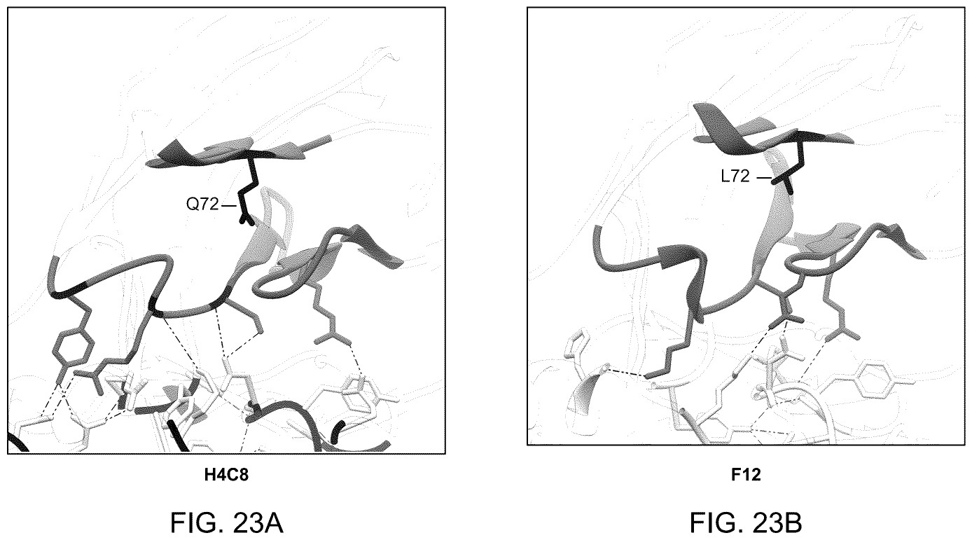

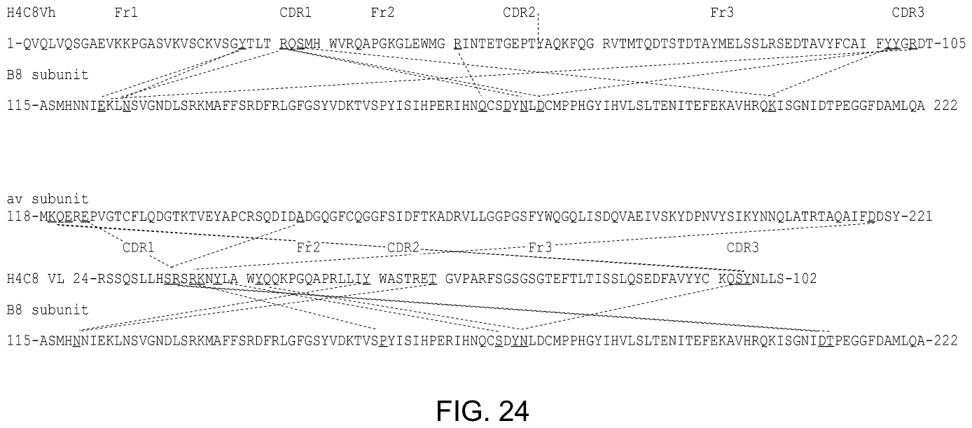

The humanized H4C8-Fab paratope and αvβ8 epitope residues that comprise the interaction interface are shown in FIGS. 21 and 22, and summarized in Table V. See also FIGS. 23 and 24. The interface is made up of van der Waals and electrostatic interactions, and corresponds to 1761.3 Å² of buried surface, as calculated by PISA. The H4C8 paratope is formed by residues from both the V_{H} and the V_{L} chain as summarized in Table VI and shown in FIGS. 22 and 24. The H4C8 Fab epitope has 100% overlap with the αv-residues that interact with L-TGF-β1 (PDB: 6UJA) and 75% overlap with the β8-residues that interact with L-TGF-β1 (PDB: 6UJA) (FIG. 22). As stated before, the epitope of the C6D4 Fab has 80% overlap with the αv-residues that interact with L-TGF-β1 (PDB: 6UJA) and 71% overlap with the β8-residues that interact with L-TGF-β1 (PDB: 6UJA). The fact that the H4C8 binding interface almost completely obstructs the binding interface of LAP of TGF-β1 residues explains why H4C8 more efficiently blocks αvβ8-mediated TGF-β activation than C6D4. The H4C8 epitope on both the av and β8-subunits is 100% conserved between mammalian species (including non-human primate, mouse, rat, sheep, cow, horse, dog, among others) explaining why H4C8 is broadly species cross-reactive. Key interacting residues (av: Glu 123, Ala 149; β8: Asn 119, Asn125, Gln168, Asp 171, Tyr 172, Asn 173, Asp 175) are not conserved between other integrin α and β subunits explaining the structural basis of the strict specificity of H4C8 for αvβ8.

### Example 11: Effector Function is not Required for Anti-β8 Inhibition of Tumor Growth

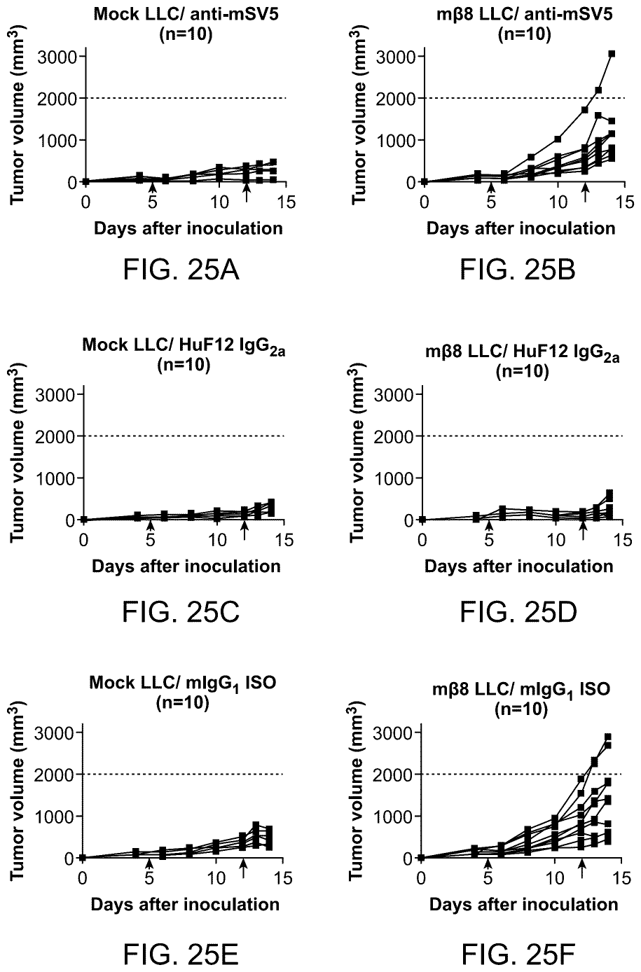

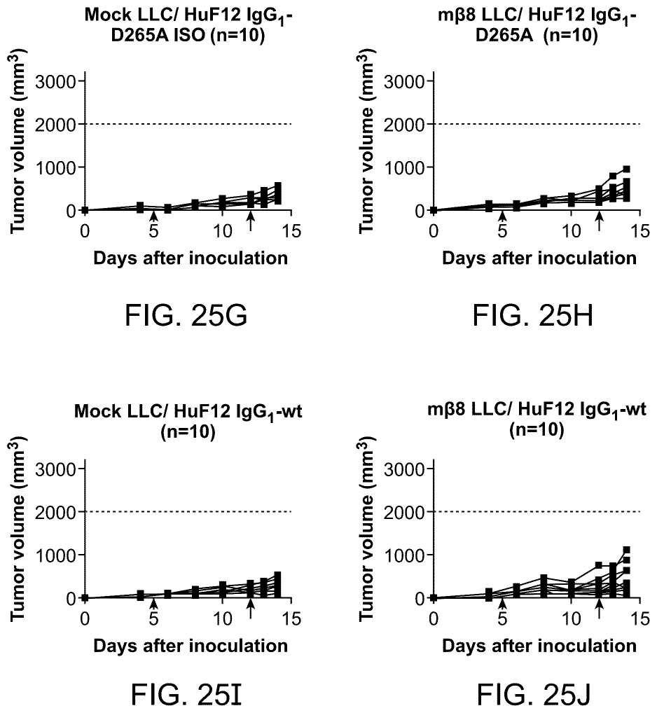

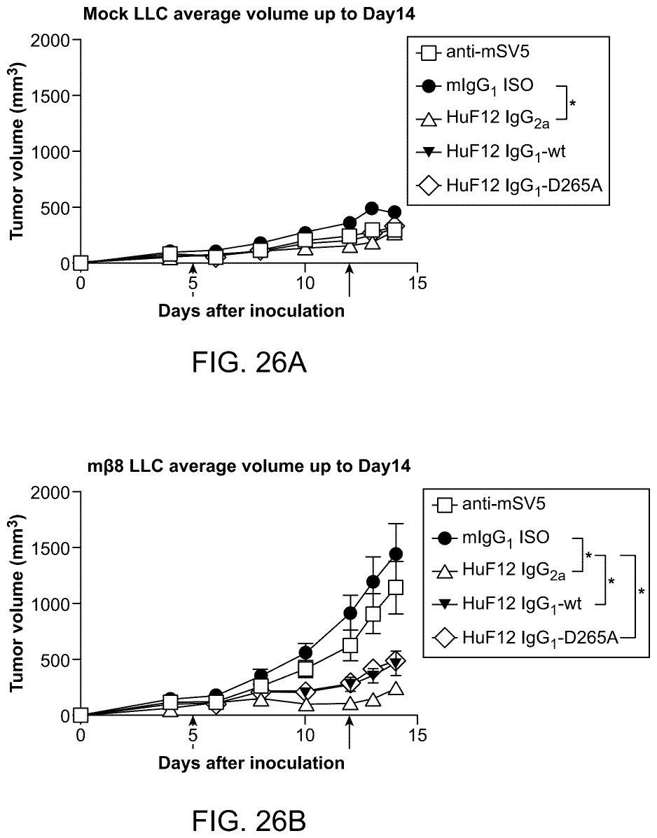

In treatment of patients with therapeutic antibodies, it is important to determine whether effector functions of antibodies are required in part or completely for the therapeutic effect. As an example of whether antibody effector function was required for the function of anti-αvβ8 antibodies directed to the HuC6D4, HuF12 and H4C8 epitope, we expressed HuF12 as a murine IgG1 (SEQ ID NO: 86). It is generally known that murine IgG1 has a similar low effector function as IgG4 does in human and can be used as a surrogate for testing whether effector function is required for antibody efficacy. These are also examples of antibodies produced with reduced effector function with specific mutations that are known in the art to reduce effector function including L235E, L234A/L235A, L234A/L235A/P329G, L234A/L235A/P329A, P331S/L234E/L235/F, G237A, D265A, D270A. In one example we expressed HuF12 (SEQ ID NO: 24, 25) fused to mouse IgG1 with the D265A mutation (SEQ ID NO: 61). C57B/6 mice were injected on one flank with Lewis lung carcinoma cells stably transfected with mouse itgb8 and sorted to uniform surface expression. On the contralateral flank mock transfected LLC cells which naturally express low level of αvβ8, were injected. Day 5 after injection, established tumors were measured, and mice were injected with anti-SV5 (IgG2a), HuF12 IgG2a, mouse IgG1 isotype control (BioXcell, MOPC-21), HuF12 mouse IgG1, or HuF12 mouse IgG1 D265A (10 mg/kg I.P) The mice were injected again on day 12 with the same antibodies. Tumor growth was measured with digital calipers. The results showed that HuF12 IgG2a was the most effective inhibitor of β8 LLC tumor growth with the F12 mouse IgG1 and F12 mouse IgG1 D265A versions having similar ability to inhibit tumor growth. These results demonstrate that antibody effector function is not required for anti-tumor efficacy of HuF12 (FIG. 25, 26).

### Example 12: Decreasing β8 Expression Decreases MC38 Tumor Growth and Improves Complete Tumor Regression Using Antibodies Directed to the HuC6D4/HuF12/H4C8 Epitope

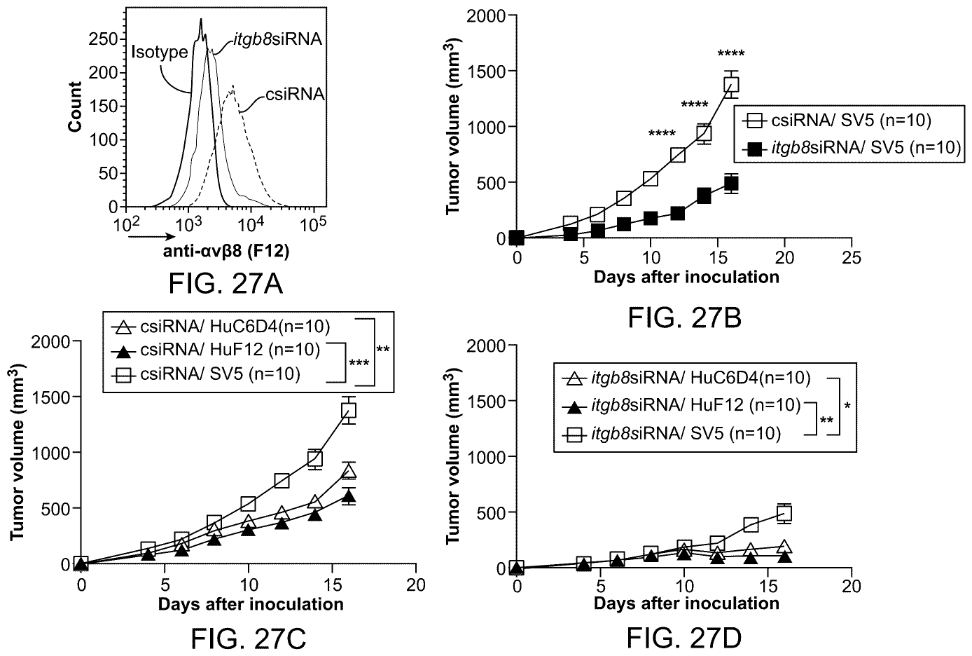

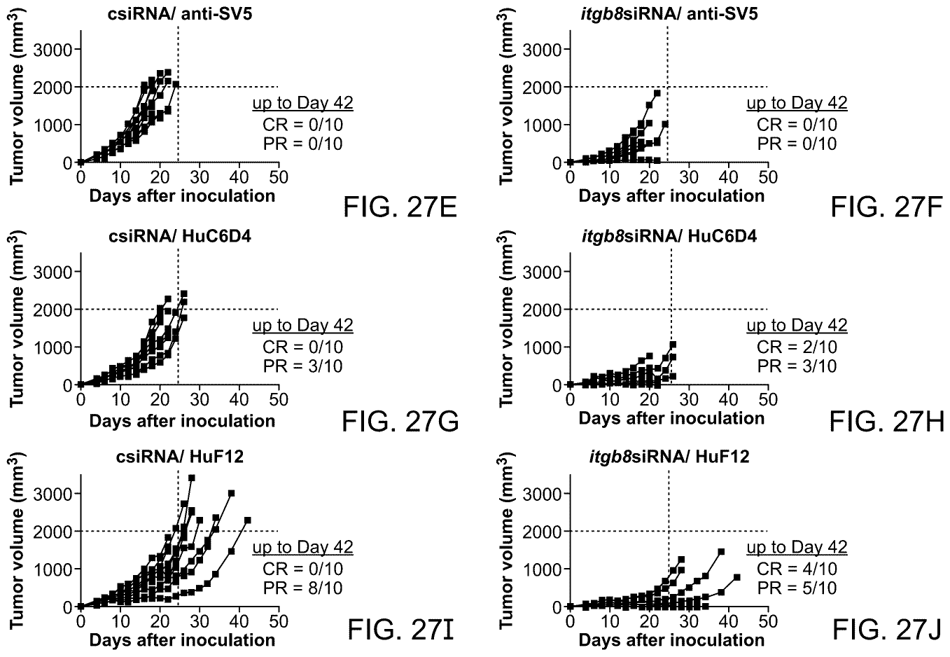

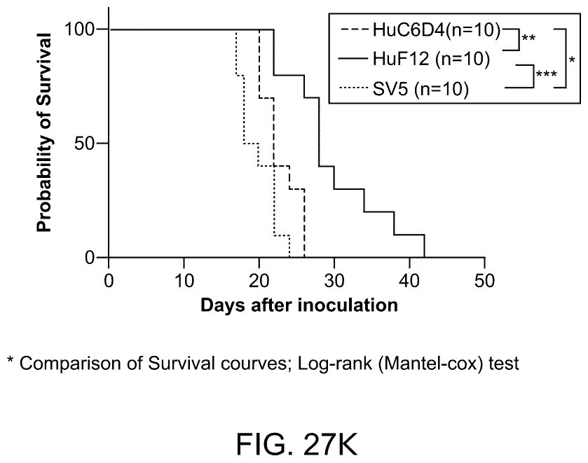

Integrin αvβ8 expressing mouse and human tumors have been shown to be enriched with immunosuppressive Treg. When 38 LLC cells were mixed with non-β8 expressing LLC cells the resulting tumors grew slower and had few Treg proportional to the percentage of with non-β8 expressing LLC cells (Seed, et al (2021) Sci. Immunol. 6, eabf0558). These data demonstrate that the level of αvβ8 expression is important in the immunosuppressive TME and tumor growth. TGF-β over-activation is an important component of resistance to checkpoint inhibitor therapy (Mariathasan, et al (2018), Nature, 554 (7693): 544-548). αvβ8-mediated TGF-β activation is important in generation of intratumoral immunosuppressive Treg and immunouppressive myeloid cells (Takasaka, et al (2018) JCI Insight (3; 20)). Therefore, we sought to test if reducing β8 expression on tumor cells would decrease tumor growth and increase responsiveness to checkpoint inhibitors. As one example, MC38 murine colon carcinoma cells were transduced with lentiviral siRNA constructs, control siRNA (csiRNA) or itgb8 siRNA (itgb8 siRNA). Transduced itgb8siRNA pools were sorted for low expression of αvβ8 and resulting pools were stained for surface αvβ8 expression using HuF12 to confirm decreased expression of αvβ8 in the itgb8 siRNA transduced cells. MC38 tumors from pools of itgb8siRNA or csiRNA MC38 cells were established on opposite flanks of C57B/6 mice, and mice treated 5 and 11 days post cell inoculation with isotype were compared with mice treated with HuC6D4 or HuF12 (10 mg/kg IP). Growth curves of MC38 tumors reveals that tumor cell expression of itgb8 increases tumorigenicity and that tumors with lower itgb8 expression are more responsive to anti-β8, with 40% of tumor with low itgb8 expression showing complete response to HuF12 IgG2a and mice with αvβ8 expressing MC38 tumors treated with HuF12 IgG2a showing statistically significant improvements in survival compared with control treated mice (FIG. 27). These data support a method whereby patients with tumors that express low levels of cell surface αvβ8 can be treated with monotherapy with antibodies targeting the HuC6D4, HuF12, H4C8 epitope.

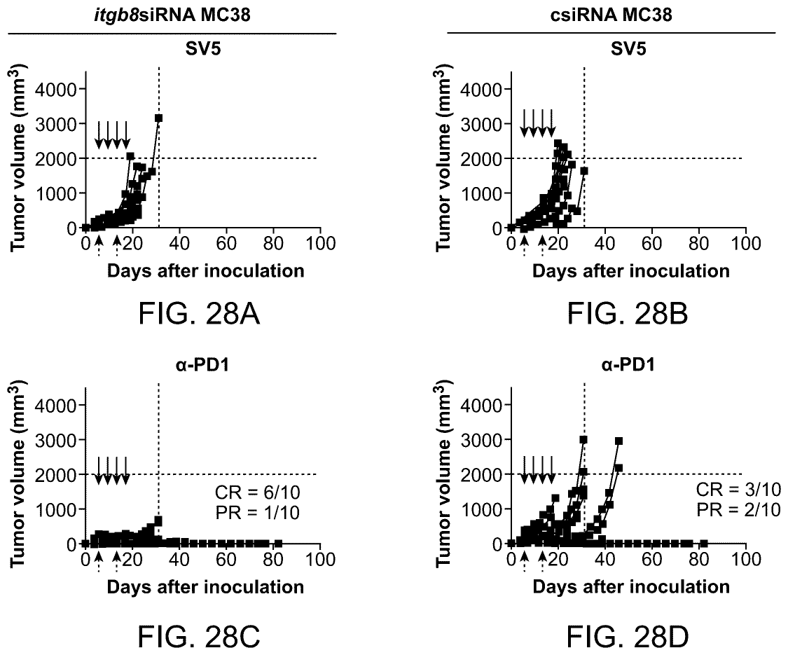

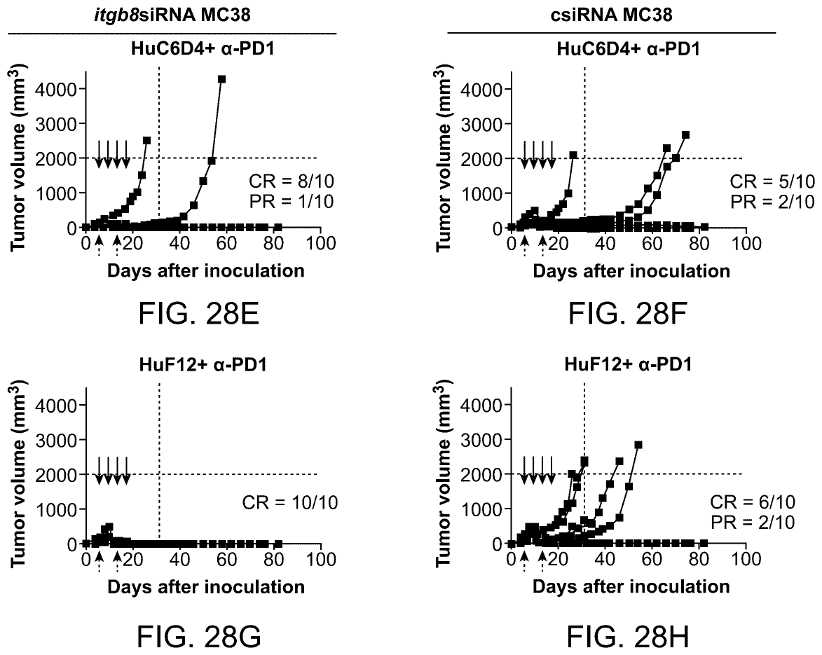

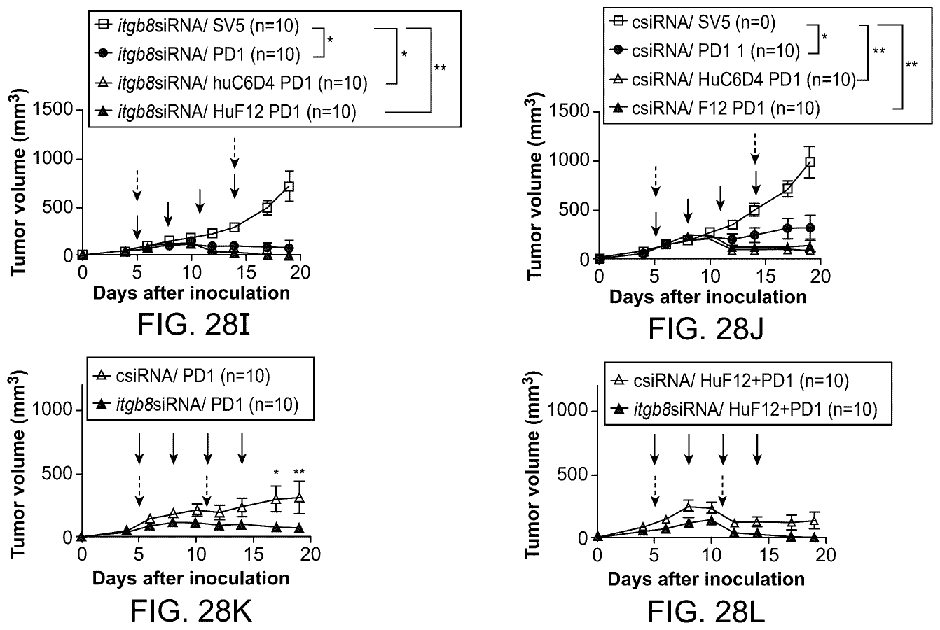

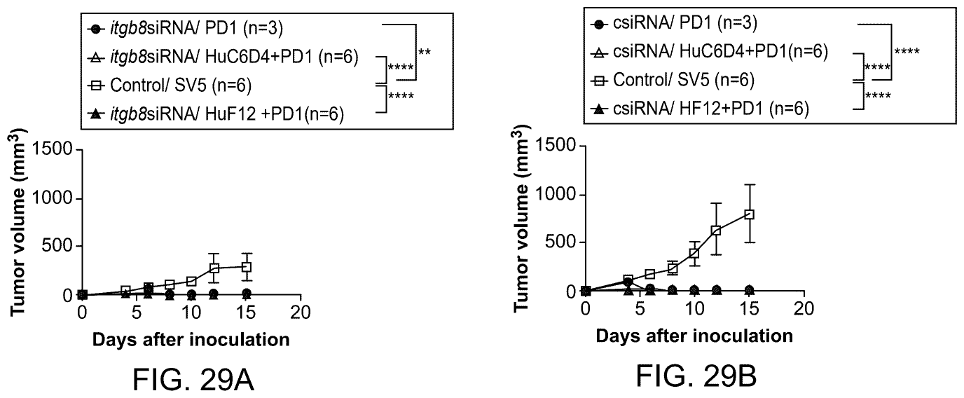

As another example, in FIG. 28, we show that HuF12 treatment shows improved tumor response in the MC38 model in combination with anti-PD1. Using the identical tumor model with lentiviral csiRNA or itgb8siRNA transduced MC38 cells and treatment with SV5 (IgG2a control), HuC6D4, HuF12, or anti-PD1 (RMPI-14, BioXCell) (10 mg/kg IP). With control and anti-avb8 antibodies injected on days 5 and 11 and anti-PD1 on days 5, 8, 11 and 14. The cumulative data shows that inhibiting αvβ8 with higher affinity anti-αvβ8 antibodies proportionately increases the effectiveness of checkpoint inhibitors with complete response seen in itgb8siRNA MC38 tumors in mice treated with the HuF12/RMPI-14 combination. These data indicate that combining a high affinity anti-αvβ8 antibody to the HuC6D4/HuF12/H4C8 epitope with a checkpoint inhibitor can lead to complete anti-tumor response which is greatest in tumors that express low levels of αvβ8. Furthermore, these immune responses are durable, since mice with complete response did not grow tumors when re-challenged (FIG. 29).

### Example 13: Identification and Characterization of Dual αvβ6/αvβ8 Inhibitors

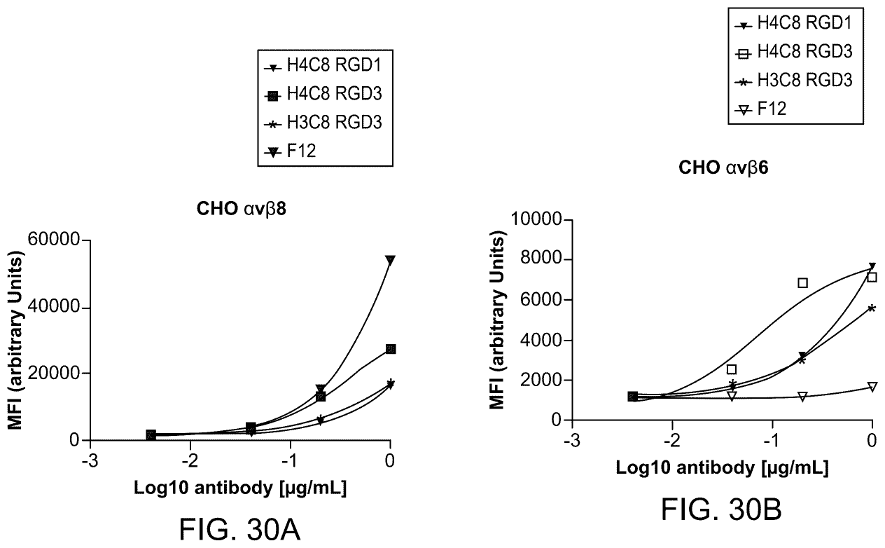

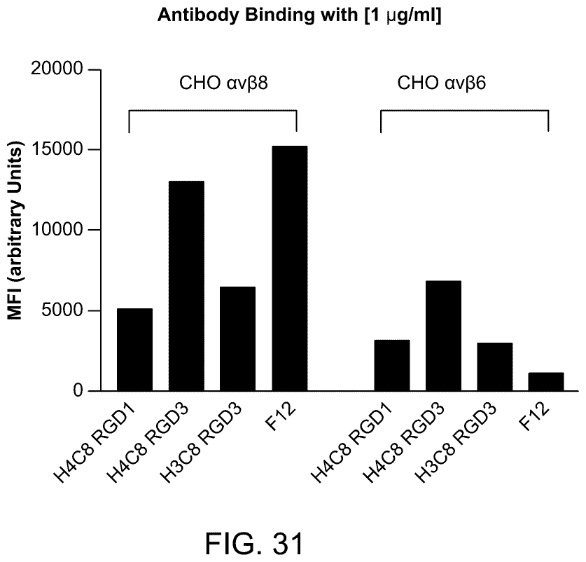

The structure of H4C8 places the VL CDR1 in the ligand binding pocket of αvβ8 where it makes multiple contacts with the αv-subunit and the specificity determining loops of the β8 subunit. As several examples of this approach, we grafted into the CDR1 VL of H4C8 (SEQ ID NO: 35) or H3.H12 (SEQ ID NO: 67), the TGF-β RGD3 loop (SEQ ID NO: 49) or the TGF-β RGD1 loop (SEQ ID NO: 50) . These VL constructs were paired as followed with heavy chains V_{H} from HuC6D4 F12 (SEQ ID NO: 24), H3.H12 (SEQ ID NO: 66), or H4C8 (SEQ ID NO: 34) in the following combinations (F12 V_{H} (SEQ ID NO: 24)+F12 RGD1 VL (SEQ ID NO: 26) antibody called F12 RGD1; H3.H12 VH (SEQ ID NO:66)+H4C8 RGD3 VL (SEQ ID NO: 40), called H3C8 RGD3; H4C8 VH (SEQ ID NO: 34)+H4C8 RGD1 V_{L} (SEQ ID NO: 44), called H4C8 RGD1; H4C8 V_{H} (SEQ ID NO: 34)+H4C8 RGD3 V_{L} (SEQ ID NO: 40), called H4C8 RGD3). These antibodies were expressed human IgG4 format and were tested for surface binding to CHO cells expressing either αvβ8 or αvβ6 (FIG. 30). H4C8 RGD3, H4C8 RGD1, and H3C8 RGD3 all bound to both integrins. At a non-saturating dose, the H4C8 RGD3 antibody bound slightly better to both αvβ8 and αvβ6 than H4C8 RGD1 (FIG. 31).

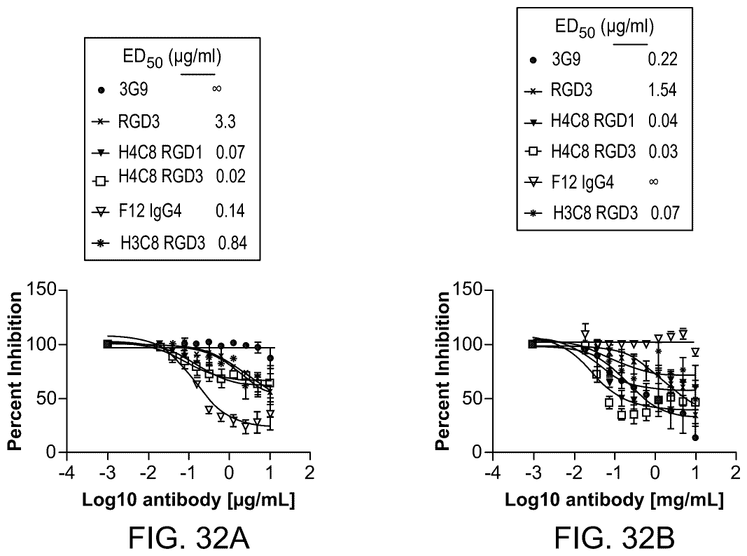

As an example of the function blocking capacity of such RGD grafted antibodies we tested the ability of H4C8 RGD3, H4C8 RGD1, F12 RGD1, H3C8 RGD3, and HuF12 to block both αvβ8- and αvβ6-mediated TGF-β activation. Shown are inhibition curves for the indicated antibody abilities to block immobilized human αvβ8 or to αvβ6 activation of human L-TGF presented on the cell surface of TMLC by GARP. The assays were performed exactly as described in (Seed et al (2021) Sci Immunol, 6, eabf0558; and Campbell, et al (2019) Cell 180 (3): 490-501). F12 RGD1 as IgG4 did not significantly inhibit activation by αvβ8 while the other antibodies H4C8 RGD1, H4C8 RGD3, H3C8 RGD3, HuC6D4 RGD3 (RGD3) and HuC6D4 F12 all blocked well in the rank order of H4C8 RGD3>H4C8 RGD1>HuC6D4 F12>H3C8 RGD3>HuC6D4 RGD3. Likewise, F12 RGD1 did not significantly inhibit activation by αvβ6 while the other antibodies H4C8 RGD1, H4C8 RGD3, H3C8 RGD3, HuC6D4 RGD3 (RGD3) and anti-β6 (3G9) all blocked well in the rank order of H4C8 RGD3>H4C8 RGD1>H3C8 RGD3>3G9>HuC6D4 RGD3. All antibodies were in human IgG4 format except RGD3 and 3G9 which were in mouse IgG2a. RGD3 consists of HuC6D4 Vh combined with HuC6D4 with the RGD3 (GRGDLGRLKK) grafted in VL CDR1 as reported in Campbell, et al (2019) Cell 180 (3): 490-501. These results prove an example that the affinity optimized humanized frameworks in H4C8 enhance the functional activities of grafted RGD ligand binding domains from TGF-β1 and TGF-β and produce antibodies with dual activity against both αvβ8- and αvβ6-mediated TGF-β activation (FIG. 32).

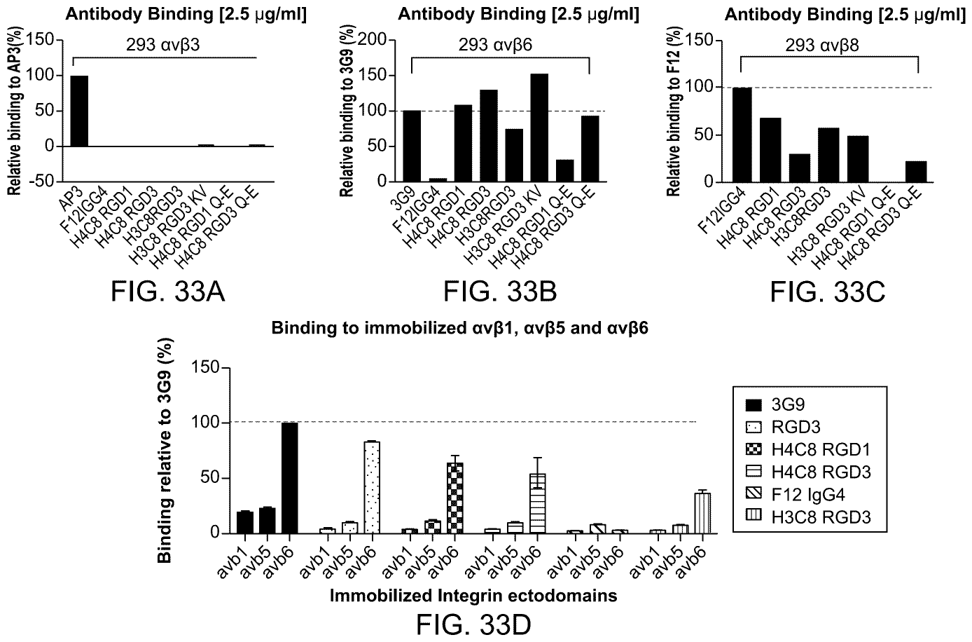

RGD ligands bind promiscuously to a number of integrins. A highly desirable quality in RGD-mimetics is to retain specificity for some but not all integrins. One highly desirable property would be a dual inhibitor to both the αvβ8- and αvβ6-integrins, without cross-reactivity to other integrins. This property would facilitate specific targeting of integrin-mediated TGF-β activation without the liability of side-effects mediated by broadly blocking other integrins. We first measured binding to αvβ3 expressing 293 cells. Binding of anti-β3 (AP3) relative to F12 IgG4, H4C8 RGD1 IgG4,H4C8 RGD3 IgG4, H3C8 RGD3 with VL Fr4 V108/L109 (mouse) IgG4, H4C8 RGD3 IgG4, H3C8 RGD3 VI Fr4 K108/V109 (human) IgG4, H4C8 RGD1 Vh Fr3 E72, H4C8 RGD3 Vh Fr3 E72 IgG4. Clearly, all antibodies showed no binding to αvβ3 compared to AP3 which bound well (FIG. 33). Expi293 cells transiently expressing αvβ6 were stained and the percentage of relative binding to the mean fluorescence intensity of anti-β6 (3G9) determined. All antibodies except HuF12 bound to αvβ6 and H3C8 RGD3 the K108/V109 in light chain Fr4 bound better than same antibody with the murine V108/L109 in in light chain Fr4. These results suggest that humanized Fr4 in the light chain is a favorable feature in these antibodies. H4C8 RGD1 Vh Fr3 E72, H4C8 RGD3 Vh Fr3 E72 bound worse than their respective versions with Vh Fr3 Q72. These results indicate that Q72 not only increases the binding interactions of H4C8 as shown in FIG. 33, but it also improves the binding interactions of H4C8 RGD1 and H4C8 RGD3 (FIG. 33). HEK 293 cells stably expressing αvβ8 were stained and HuF12 staining was compared to the other antibodies, which all bound except H4C8 RGD1 Vh Fr3 E72. The antibodies (F12 IgG4, H4C8 RGD1 IgG4,H4C8 RGD3 IgG4, H3C8 RGD3 with VI Fr4 V108/L109 (mouse) IgG4, H4C8 RGD3 IgG4, H3C8 RGD3 VI Fr4 K108/V109 (human) IgG4, H4C8 RGD1 Vh Fr3 E72, H4C8 RGD3 Vh Fr3 E72 IgG4) were next tested for their abilities to bind to αvβ1 and αvβ5 integrins. Ectodomains of these integrins did not bind to any of the antibodies, while they all bound to αvβ6, except HuF12. The positive control anti-β6 (3G9) bound well to αvβ6 (FIG. 33). These results indicate that H4C8 RGD1 and H4C8 RGD3 are highly specific dual inhibitors of the αvβ8- and αvβ6-integrins, and have better inhibitory properties than their chimeric (HuF12) counterpart.

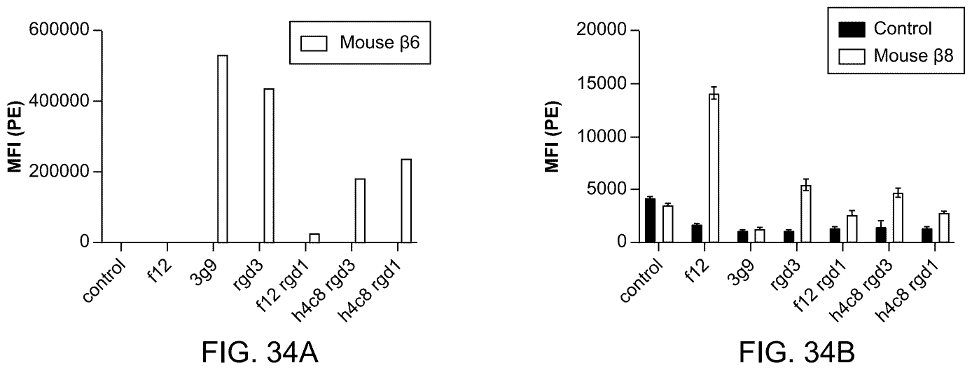

A key property for therapeutic development is cross-species reactivity. This allows preclinical testing of antibodies in small and large animals. As an example of species cross reactivity we tested the H4C8 RGD1, H4C8 RGD3 antibodies for cross reactivity to mouse. As shown in FIG. 34, H4C8 RGD1, H4C8 RGD3 antibodies both bound to mouse αvβ8 and αvβ6 indicating cross-reactivity (FIG. 34).

### Example 14: The 68 Integrin Protein and ITGB8 Transcript are Biomarkers of Checkpoint Inhibitor Resistance

Immunobiologists and pathologists have long recognized that tumors are infiltrated by cells of both the innate and adaptive arms of the immune system and thereby mirror inflammatory conditions arising in non-neoplastic environments. The tumor microenvironment (TME) is a complex environment where malignant cells interact with both immune and nonimmune cells. Cancer immunotherapy has revolutionized cancer care by acting directly on cells with the TME and re-engaging the anti-tumor immune response. While current immunotherapies have led to anti-tumor response in a subset of cancer patients, the vast majority of cancers show little if no response to currently available therapies.

Robust anti-tumor responses to immunotherapy are only rarely seen across multiple tumor types. Different cells and proteins within the TME appear to inhibit the effectiveness of immunotherapies in particular TGF-β, which is one of the most potent immunosuppressive molecules known. A TGF-β signature correlates with poor outcome, lack of response to immunotherapy, and exclusion of immune cells from tumor centers (Mariathasan, et al (2018), Nature, 554 (7693): 544-548).

Biomarkers that can predict responsiveness to immunotherapy, in particular biomarkers to the TGF-β pathway, are essential to improve patient selection for immunotherapeutic treatment regimens. Key to biomarker development is to understand the underlying basis for tumor heterogeneity. As one example, patterns of immune cell infiltration serve a biomarker that predicts tumor response to immunotherapy. Rather than focusing on a single immune cell type, a given immune response can be conceived as a collection of cell subsets and specific immune cell pairings linked with function allowing us to define “immune archetypes”. It is likely that individual tumors engage common patterns of the immune system—here ‘Archetypes’—creating prototypical patterns of immune anti-tumor immune responses. In Combes, et al (2022) Cell, (5, 420:123-127), we leveraged a unique dataset composed of both cell type compositional and transcriptomic data from 364 fresh surgical specimens across 12 tumor types (Bladder, Colorectal, Glioblastoma, Gynecologic, Liver, Head and Neck, Kidney, Lung, Skin, Pancreas, Sarcoma) to identify conserved tumor immune archetypes. We used an unsupervised clustering approach based on tumor specific immune gene signatures, benchmarked against ‘ground truth’ cell type composition data from flow cytometry, to identify and validate 12 unique tumor immune archetypes that were also identified in the TCGA dataset. These archetypes, discovered with only ten features, differentially aggregated other cell types that were not part of the discovery features, and delineate immune and tumor transcriptomic programs across different tissues.

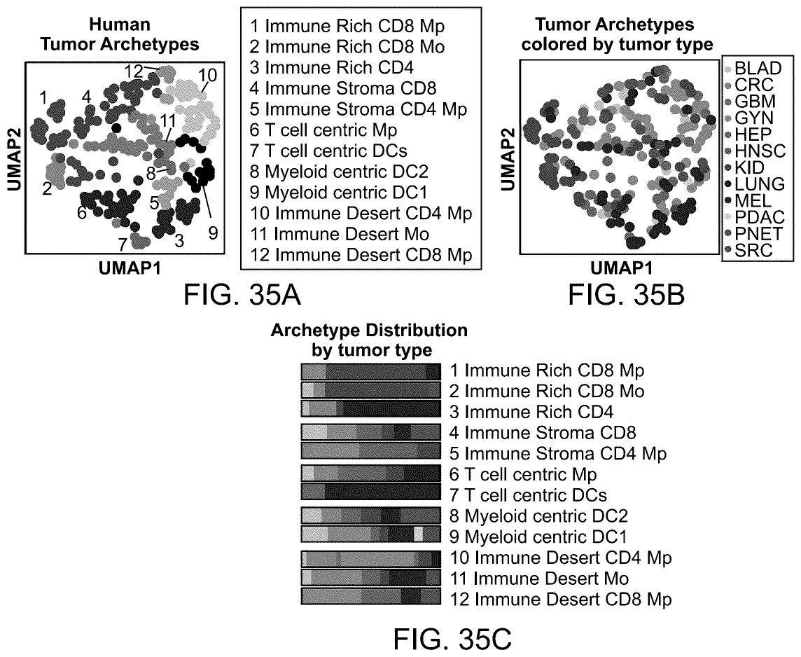

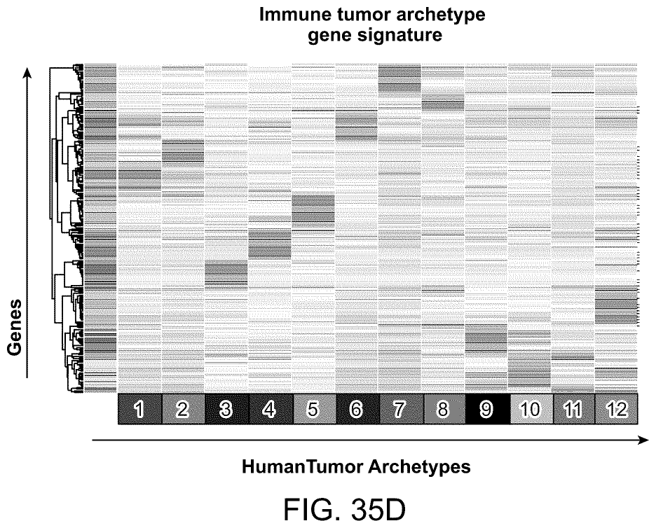

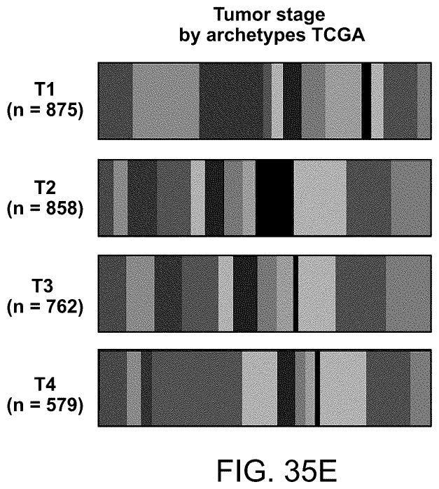

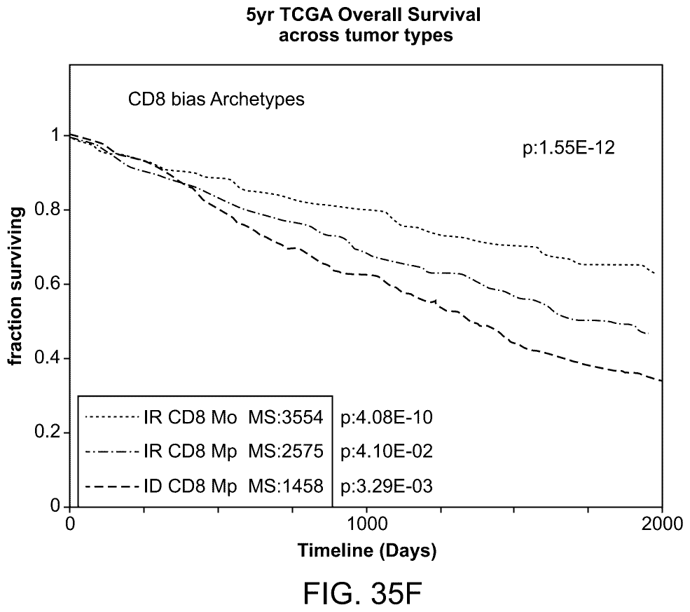

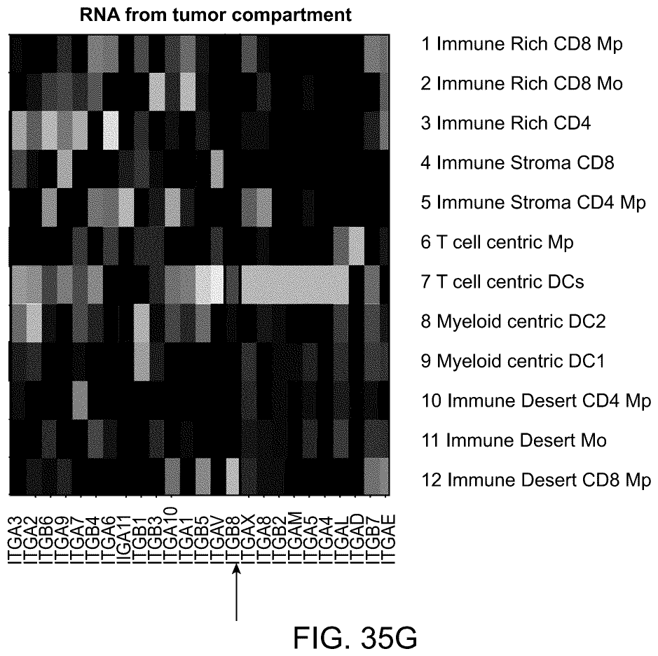

The 12 unique tumor “archetypes” represent the spectrum of immune-rich to immune-desert and reflect distinct immune networks and tumor transcriptomic programs for each archetype with unique relationships among immune cells and chemokine networks (FIG. 35). The tumors archetypes provide a distinct immune classification that is distinct from individual tumor types (FIG. 35). Archetype gene signatures were generated by differential gene expression analysis, between the live compartment counts of samples in each of the 12 archetypes, using Limma and Voom. A hierarchical clustered heatmap was made to visualize archetype gene signature gene expression. The median TPM expression per archetype of each of the genes in the gene signatures were clustered using correlation distance metric and the average linkage method (FIG. 35). Those signatures were then used to identify the same tumor archetypes in a TCGA (https://www.cancer.gov/about-nci/organization/ccg/research/structural-genomics/tcga) dataset composed of tumor samples from more than 4000 tumor patients. While the 12 archetypes were spread across both tumor type and stage in the TCGA dataset (FIG. 35), using multivariate analysis across tumors, we detected significant outcome differences between archetypes that have similar T cell subset enrichment regardless of the tumor type and tissue origin. For instance, in CD8+ T cells biased archetypes, the Immune Desert Macrophage bias archetype (full black line) presents a significantly worst outcome than immune rich archetypes-(dashed black and dashed grey line) (FIG. 35). Using hierarchical clustering of the median integrin subunit expression among the tumors of each archetype, ITGB8 stood out from all other integrins studied as being highly expressed in immune desert archetypes especially the Immune Desert Macrophage bias archetype 12 (FIG. 35). This archetype contains melanoma, gynecologic, head and neck carcinoma, and sarcomas. These data indicate that ITGB8 is biomarker of the Immune Desert Macrophage bias archetype 12. Since transcriptional regulation of ITGB8 determines αvβ8 protein expression in cells, certain threshold values of ITGB8 can be used as a surrogate for αvβ8 protein expression (Markovics, et al (2010), J Biol Chem 285 (32) P24695-24706). Thus, tumors which express the ITGB8 gene product and/or display immune exclusion could benefit from a treatment targeting integrin αvβ8.

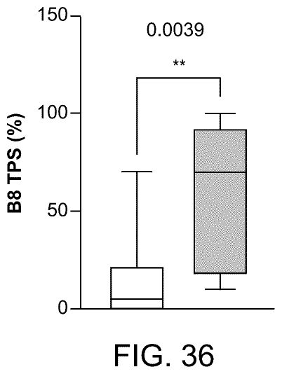

As an example of using expression of the integrin αvβ8 protein as a biomarker to guide therapy, in FIG. 36, tumor samples from patients with non-small cell carcinoma that were treated with front-line checkpoint inhibitor (monotherapy) were stained for integrin αvβ8 using clone F9 (Takasaka et al (2018) JCI Insight, 3 (20)). The patients were categorized as non-responders (grey bars, n=6) versus partial responders (open bars, n=14). Antibody staining was characterized as positive with membrane staining, or negative by the absence of staining. Immunohistochemical staining was performed using the Leica (Bond-III) automated staining platform. Tumor proportion scoring (TPS) was performed and interpreted as described for PD-L1 staining using clone 22C3 (www.agilent.com).

As another example of the spectrum of patients that could benefit from treatment with and anti-β8 antibody, we found that β8 is broadly expressed in subsets of human cancers. We have performed exhaustive surveys of tissue expression using a clone F9 on formalin fixed paraffin embedded tissue and have found β8 expression broadly expressed in epithelial derived tumors (lung squamous, adenocarcinoma, ovarian, breast, endometrioid, skin squamous, prostatic, colon, gastric and upper aerodigestive cancers) (Takasaka, et al (2018) JCI Insight, 3 (20). We and other have also detected ITGB8 in both tumor an stromal cells in various cancer and tissue types including Ovarian, Lung and Head and Neck (Seed, et al (2021) Sci. Immunol. 6, eabf0558), as well in human lung fibroblasts (Kitamura, et al (2011), J Clin Invest 121 (7): 2863-2875), as well as brain tumors (Guerro, et al (2017) Oncogene 36:6568-6580). Finally, a number of studies also find expression on αvβ8 on certain immunosuppressive immune cell subsets including human myeloid cells (Kelly, et al (2018) J Exp Med, 215 (11): 2725-2736), and Treg cells (Stockis, et al (2017) Proc Acad Sci USA, 114 (47)). Together these results demonstrate that not only is β8 expressed by tumor cells in subsets of a wide variety of tumor types, it is also expressed widely in many cell types in the TME indicating that patients may be selected on the basis of β8 expression in any component of the tumor or TME.

Many modifications and variations of this invention can be made without departing from its spirit and scope, as will be apparent to those skilled in the art. The specific embodiments described herein are offered by way of example only and are not meant to be limiting in any way.

All documents (for example, patents, patent applications, books, journal articles, or other publications) cited herein are incorporated by reference in their entirety and for all purposes, to the same extent as if each individual document was specifically and individually indicated to be incorporated by reference in its entirety for all purposes. To the extent such documents incorporated by reference contradict the disclosure contained in the specification, the specification is intended to supersede and/or take precedence over any contradictory material.

SEQUENCES

RQSMH (SEQ ID NO: 1)
RINTETGEPTYAQKFQG (SEQ ID NO: 2)
FYYGRD(S/T) (SEQ ID NO: 3)
RSSQSLLHSRSRKNYLA (SEQ ID NO: 4)
WASTRET (SEQ ID NO: 5)
KQSYNLLS (SEQ ID NO: 6)
EQSMH (SEQ ID NO: 7)
KSSQSLLGRGDLGRLKKNALA (SEQ ID NO: 8)
RSSQSLLGRGDLGRLKKNALA (SEQ ID NO: 9)
KSSQSLLRRGDLATIHGNALA (SEQ ID NO: 10)
RSSQSLLRRGDLATIHGNALA (SEQ ID NO: 11)
QVQLVQSGAEVKKPGASVKVSCKVSGYTLT (SEQ ID NO: 12)
WVRQAPGKGLEWMG (SEQ ID NO: 13)
RVTMTQDTSTDTAYMELSSLRSEDTAVYFCAI (SEQ ID NO: 14)
WGQGTTVTVSS (SEQ ID NO: 15)
EIVMTQSPATLSVSPGERVTLSC (SEQ ID NO: 16)
WYQQKPGQAPRLLIY (SEQ ID NO: 17)
GVPARFSGSGSGTEFTLTISSLQSEDFAVYYC (SEQ ID NO: 18)
FGQGTKVEIK (SEQ ID NO: 19)
FGQGTVLEIK (SEQ ID NO: 20)
RVTMTEDTSTDTAYMELSSLRSEDTAVYFCAI (SEQ ID NO: 21)
huC6D4 VH
SEQ ID NO: 22:
QIQLVQSGAEVKKPGASVKISCKASGYTFTDYSMHWVRQAPGQGLEWVARINTETGEPTFADDF
RGRFTVTLDTSTSTAYLEIRSLRSDDTAVYFCAIFYYGRDTWGQGTTLTVSS
huC6D4 VL
SEQ ID NO: 23:
EIVMTQSPATLSVSPGERVTMSCKSSQSLLNSRSRKNYLAWYQQKPGQAPRLLIYWASTRESGV
PARFSGSGSGTEFTLTISSVQSEDFAVYYCKQSYNLLSFGQGTVLEIKR
F12 VH
SEQ ID NO: 24:
QIQLVQSGAEVKKPGASVKISCKASGYTFTKYSMHWVRQAPGQGLEWVARINTETGEPTFADDF
RGRFTVTLDTSTSTAYLEIRSLRSDDTAVYFCAIFYYGRDTWGQGTTLTVSS
F12 VL
SEQ ID NO: 25:
EIVMTQSPATLSVSPGERVTMSCKSSQSLLHSRSRKNYLAWYQQKPGQAPRLLIYWASTRESGV
PARFSGSGSGTEFTLTISSVQSEDFAVYYCKQSYNLLSFGQGTVLEIKR
TGFb1RGD VL [See previous SEQ ID NO: 12 from WO 2021/146614]
SEQ ID NO: 26
DIVMTQSPSSLAVSAGEKVTMSCKSSQSLLRRGDLATIHGNALAWYQQKPGQSPRLLIYWASTR
ESGVPDRFTGSGSGTDFTLTISSVQAEDLAVYYCKQSYNLLSFGAGTKLELKA
C6D4-RGD3 VL [See previous SEQ ID NO: 39 from WO 2021/146614; and
SEQ ID NO: 492 from WO2018/064478 (C6D4-RGD3)]
SEQ ID NO: 27:
DIVMTQSPSSLAVSAGEKVTMSCKSSQSLLGRGDLGRLKKNALAWYQQKPGQSPRLLIYWASTR
ESGVPDRFTGSGSGTDFTLTISSVQAEDLAVYYCKQSYNLLSFGAGTKLELKA
HuC6D4-RGD3 VL [See previous SEQ ID NO: 500 from WO2018/064478]
SEQ ID NO: 28:
EIVMTQSPATLSVSPGERVTMSCKSSQSLLGRGDLGRLKKNALAWYQQKPGQAPRLLIYWASTR
ESGVPARFSGSGSGTEFTLTISSVQSEDFAVYYCKQSYNLLSFGQGTVLEIKR
heavy_IGHV1-24_IGHD5-18_IGHJ6, partial [Homo sapiens]
SEQ ID NO: 29:
QVQLVQSGAEVKKPGASVKVSCKVSGYTLTELSMHWVRQAPGKGLEWMGREDPEDGETIYAQKF
QGRVTMTEDISTDTAYMELSTLRSEDTAVYYCATGVWIREDYGMDVWGQGTTVTVSS
QEP17497.1 IGH c3222_heavy_IGHV1-24_IGHD3-9_IGHJ5, partial [Homo sapiens]
SEQ ID NO: 30:
QVQLVQSGAEVKKPGASVKVSCKVSGYTLTELSMHWVRQAPGKGLEWMGGEDPEDGETIYAQKF
QGRVTMTEDTSTDTAYMELSSLRSEDTAVYYCATVVRYFDWSIGGWGQGTLVTVSS
IGHV1-24*01
SEQ ID NO: 31:
QVQLVQSGAEVKKPGASVKVSCKVSGYTLTELSMHWVRQAPGKGLEWMGGEDPEDGETIYAQKF
QGRVTMTEDTSTDTAYMELSSLRSEDTAVYYCAT
>QEP13372.1 IGL c3311_light_IGKV3-15_IGKJ1, partial [Homo sapiens]
SEQ ID NO: 32:
EIVMTQSPATLSVSPGERATLSCRASQSVSSNLAWYQQKPGQAPRLLIYGASTRATGIPARFSG
SGSGTEFTLTISSLQSEDFAVYYCQQSETFGQGTKVEIK
IGKV3-15*01
SEQ ID NO: 33:
EIVMTQSPATLSVSPGERATLSCRASQSVSSNLAWYQQKPGQAPRLLIYGASTRATGIPARFSG
SGSGTEFTLTISSLQSEDFAVYYC
H4.C8 (VL mutated in FR4 to KV) Italics = differences in CDRs
from F12 underline = Framework different from F12
SEQ ID NO: 34:
QVQLVQSGAEVKKPGASVKVSCKVSGYTLTRQSMHWVRQAPGKGLEWMGRINTETGEPTYAQKF
QGRVTMTQDTSTDTAYMELSSLRSEDTAVYFCAIFYYGRDTWGQGTTVTVSS
SEQ ID NO: 35:
EIVMTQSPATLSVSPGERVTLSCRSSQSLLHSRSRKNYLAWYQQKPGQAPRLLIYWASTRETGV
PARFSGSGSGTEFTLTISSLQSEDFAVYYCKQSYNLLSFGQGTKVEIKR
H4.C8 (KV FR4 VL mutation) Italic text = mouse residues
SEQ ID NO: 34:
QVQLVQSGAEVKKPGASVKVSCKVSGYTLTRQSMHWVRQAPGKGLEWMGRINTETGEPTYAQKF
QGRVTMTQDTSTDTAYMELSSLRSEDTAVYFCAIFYYGRDTWGQGTTVTVSS
SEQ ID NO: 36:
EIVMTQSPATLSVSPGERVTLSCRSSQSLLHSRSRKNYLAWYQQKPGQAPRLLIYWASTRET
GVPARFSGSGSGTEFTLTISSLQSEDFAVYYCKQSYNLLSFGQGTVLEIKR
H4.C8 (VH FR2 Q to E mutation) Italic text = Q to E (human)
mutation; Q mutation increases affinity and expression, E
balances charge distribution
SEQ ID NO: 37:
QVQLVQSGAEVKKPGASVKVSCKVSGYTLTRQSMHWVRQAPGKGLEWMGRINTETGEPTYAQKF
QGRVTMTEDTSTDTAYMELSSLRSEDTAVYFCAIFYYGRDTWGQGTTVTVSS
SEQ ID NO: 35:
EIVMTQSPATLSVSPGERVTLSCRSSQSLLHSRSRKNYLAWYQQKPGQAPRLLIYWASTRET
GVPARFSGSGSGTEFTLTISSLQSEDFAVYYCKQSYNLLSFGQGTKVEIKR
H4.C8 (VH FR2 Q to E mutation) Italic text = Q to E mutation; Q
mutation increases affinity and expression; (KV FR4 to VL
mutation) Italic text = mouse residues
SEQ ID NO: 37:
QVQLVQSGAEVKKPGASVKVSCKVSGYTLTRQSMHWVRQAPGKGLEWMGRINTETGEPTYAQKF
QGRVTMTEDTSTDTAYMELSSLRSEDTAVYFCAIFYYGRDTWGQGTTVTVSS
SEQ ID NO: 36
EIVMTQSPATLSVSPGERVTLSCRSSQSLLHSRSRKNYLAWYQQKPGQAPRLLIYWASTRETGV
PARFSGSGSGTEFTLTISSLQSEDFAVYYCKQSYNLLSFGQGTVLEIKR
H3.C8 (VL FR4 to KV mutation) Italic text = differences in CDRs from F12
SEQ ID NO: 38:
QVQLVQSGAEVKKPGASVKVSCKVSGYTLTEQSMHWVRQAPGKGLEWMGRINTETGEPTYAQKF
QGRVTMTQDTSTDTAYMELSSLRSEDTAVYFCAIFYYGRDTWGQGTTVTVSS
SEQ ID NO: 35:
EIVMTQSPATLSVSPGERVTLSCRSSQSLLHSRSRKNYLAWYQQKPGQAPRLLIYWASTRETGV
PARFSGSGSGTEFTLTISSLQSEDFAVYYCKQSYNLLSFGQGTKVEIKR
H3.C8 (KV FR4 to VL mutation) Italic text = mouse residues in light chain
SEQ ID NO: 38:
QVQLVQSGAEVKKPGASVKVSCKVSGYTLTEQSMHWVRQAPGKGLEWMGRINTETGEPTYAQKF
QGRVTMTQDTSTDTAYMELSSLRSEDTAVYFCAIFYYGRDTWGQGTTVTVSS
SEQ ID NO: 36:
EIVMTQSPATLSVSPGERVTLSCRSSQSLLHSRSRKNYLAWYQQKPGQAPRLLIYWASTRETGV
PARFSGSGSGTEFTLTISSLQSEDFAVYYCKQSYNLLSFGQGTVLEIKR
H3.C8 (VH FR2 Q to E mutation) Italic text = Q to E (human)
mutation; Q mutation increases affinity and expression, E
balances charge distribution
SEQ ID NO: 39:
QVQLVQSGAEVKKPGASVKVSCKVSGYTLTEQSMHWVRQAPGKGLEWMGRINTETGEPTYAQKF
QGRVTMTEDTSTDTAYMELSSLRSEDTAVYFCAIFYYGRDTWGQGTTVTVSS
SEQ ID NO: 35
EIVMTQSPATLSVSPGERVTLSCRSSQSLLHSRSRKNYLAWYQQKPGQAPRLLIYWASTRETGV
PARFSGSGSGTEFTLTISSLQSEDFAVYYCKQSYNLLSFGQGTKVEIKR
H4.C8 (VH FR2 Q to E mutation) Italic text = Q to E (human)
mutation; Q mutation increases affinity and expression, E
balances charge distribution; (KV FR4 to VL mutation) Italic
text = mouse residues in SEQ ID NO: 36
SEQ ID NO: 39:
QVQLVQSGAEVKKPGASVKVSCKVSGYTLTEQSMHWVRQAPGKGLEWMGRINTETGEPTYAQKF
QGRVTMTEDTSTDTAYMELSSLRSEDTAVYFCAIFYYGRDTWGQGTTVTVSS
SEQ ID NO: 36:
EIVMTQSPATLSVSPGERVTLSCRSSQSLLHSRSRKNYLAWYQQKPGQAPRLLIYWASTRETGV
PARFSGSGSGTEFTLTISSLQSEDFAVYYCKQSYNLLSFGQGTVLEIKR
H4.C8 RGD3 K (VK FR4 KV) VK CDR1 RGD3 from WO2018/064478
SEQ ID NO: 34:
QVQLVQSGAEVKKPGASVKVSCKVSGYTLTRQSMHWVRQAPGKGLEWMGRINTETGEPTYAQKF
QGRVTMTQDTSTDTAYMELSSLRSEDTAVYFCAIFYYGRDTWGQGTTVTVSS
SEQ ID NO: 40:
EIVMTQSPATLSVSPGERVTLSCKSSQSLLGRGDLGRLKKNALAWYQQKPGQAPRLLIYWASTR
ETGVPARFSGSGSGTEFTLTISSLQSEDFAVYYCKQSYNLLSFGQGTKVEIKR
H4.C8 RGD3 K (KV FR4 to VL mutation) Italic text = mouse
residues in SEQ ID NO: 41
SEQ ID NO: 34:
QVQLVQSGAEVKKPGASVKVSCKVSGYTLTRQSMHWVRQAPGKGLEWMGRINTETGEPTYAQKF
QGRVTMTQDTSTDTAYMELSSLRSEDTAVYFCAIFYYGRDTWGQGTTVTVSS
SEQ ID NO: 41:
EIVMTQSPATLSVSPGERVTLSCKSSQSLLGRGDLGRLKKNALAWYQQKPGQAPRLLIYWASTR
ETGVPARFSGSGSGTEFTLTISSLQSEDFAVYYCKQSYNLLSFGQGTVLEIKR
H4.C8 RGD3 K (VH FR2 Q to E mutation) Italic text = Q to E
mutation; Q mutation increases affinity and expression, E
balances charge distribution
SEQ ID NO: 37:
QVQLVQSGAEVKKPGASVKVSCKVSGYTLTRQSMHWVRQAPGKGLEWMGRINTETGEPTYAQKF
QGRVTMTEDTSTDTAYMELSSLRSEDTAVYFCAIFYYGRDTWGQGTTVTVSS
SEQ ID NO: 40:
EIVMTQSPATLSVSPGERVTLSCKSSQSLLGRGDLGRLKKNALAWYQQKPGQAPRLLIY
WASTRETGVPARFSGSGSGTEFTLTISSLQSEDFAVYYCKQSYNLLSFGQGTKVEIKR
H4 C8 RGD3 K (VH FR2 Q to E; KV FR4 VL mutation) Italic text =
Q to E (human) mutation; Q mutation increases affinity and
expression, E balances charge distribution; Italic text = mouse
residues in SEQ ID NO: 41
SEQ ID NO: 37:
QVQLVQSGAEVKKPGASVKVSCKVSGYTLTRQSMHWVRQAPGKGLEWMGRINTETGEPTYAQKF
QGRVTMTEDTSTDTAYMELSSLRSEDTAVYFCAIFYYGRDTWGQGTTVTVSS
SEQ ID NO: 41:
EIVMTQSPATLSVSPGERVTLSCKSQSLLGRGDLGRLKKNALAWYQQKPGQAPRLLIYWASTRE
TGVPARFSGSGSGTEFTLTISSLQSEDFAVYYCKQSYNLLSFGQGTVLEIKR
H4.C8 RGD3 R (VL FR4 to KV mutation)
SEQ ID NO: 38:
QVQLVQSGAEVKKPGASVKVSCKVSGYTLTEQSMHWVRQAPGKGLEWMGRINTETGEPTYAQKF
QGRVTMTQDTSTDTAYMELSSLRSEDTAVYFCAIFYYGRDTWGQGTTVTVSS
SEQ ID NO: 42:
EIVMTQSPATLSVSPGERVTLSCRSSQSLLGRGDLGRLKKNALAWYQQKPGQAPRLLIYWASTR
ETGVPARFSGSGSGTEFTLTISSLQSEDFAVYYCKQSYNLLSFGQGTKVEIKR
H4.C8 RGD3 R (KV FR4 to VL mutation) Italic text = mouse residues
SEQ ID NO: 38:
QVQLVQSGAEVKKPGASVKVSCKVSGYTLTEQSMHWVRQAPGKGLEWMGRINTETGEPTYAQKF
QGRVTMTQDTSTDTAYMELSSLRSEDTAVYFCAIFYYGRDTWGQGTTVTVSS
SEQ ID NO: 43:
EIVMTQSPATLSVSPGERVTLSCRSSQSLLGRGDLGRLKKNALAWYQQKPGQAPRLLIYWASTR
ETGVPARFSGSGSGTEFTLTISSLQSEDFAVYYCKQSYNLLSFGQGTVLEIKR
H4.C8 RGD3 R (VH FR2 Q to E mutation; VL FR4 to KV
mutation) Italic text = Q to E (human) mutation; Q mutation
increases affinity and expression, E balances charge distribution
SEQ ID NO: 39:
QVQLVQSGAEVKKPGASVKVSCKVSGYTLTEQSMHWVRQAPGKGLEWMGRINTETGEPTYAQKF
QGRVTMTEDTSTDTAYMELSSLRSEDTAVYFCAIFYYGRDTWGQGTTVTVSS
SEQ ID NO: 42:
EIVMTQSPATLSVSPGERVTLSCRSSQSLLGRGDLGRLKKNALAWYQQKPGQAPRLLIYWASTR
ETGVPARFSGSGSGTEFTLTISSLQSEDFAVYYCKQSYNLLSFGQGTKVEIKR
H4.C8 RGD3 R (VH FR2 Q to E mutation; KV FR4 to VL
mutation) Italtic text = Q to E mutation; Q mutation increases
affinity and expression, E balances charge distribution; Italic
text = mouse residues in SEQ ID NO: 43
SEQ ID NO: 39:
QVQLVQSGAEVKKPGASVKVSCKVSGYTLTEQSMHWVRQAPGKGLEWMGRINTETGEPTYAQKF
QGRVTMTEDTSTDTAYMELSSLRSEDTAVYFCAIFYYGRDTWGQGTTVTVSS
SEQ ID NO: 43:
EIVMTQSPATLSVSPGERVTLSCRSSQSLLGRGDLGRLKKNALAWYQQKPGQAPRLLIYWASTR
ETGVPARFSGSGSGTEFTLTISSLQSEDFAVYYCKQSYNLLSFGQGTVLEIKR
H4. C8 RGD1 K (VL FR4 to KV mutation) CDRVK1 RGD1 from WO2021/146614
SEQ ID NO: 34:
QVQLVQSGAEVKKPGASVKVSCKVSGYTLTRQSMHWVRQAPGKGLEWMGRINTETGEPTYAQKF
QGRVTMTQDTSTDTAYMELSSLRSEDTAVYFCAIFYYGRDTWGQGTTVTVSS
SEQ ID NO: 44:
EIVMTQSPATLSVSPGERVTLSCKSSQSLLRRGDLATIHGNALAWYQQKPGQAPRLLIYWASTR
ETGVPARFSGSGSGTEFTLTISSLQSEDFAVYYCKQSYNLLSFGQGTKVEIKR
H4.C8 RGD1 K (KV FR4 to VL mutation) Italic text = mouse residues
SEQ ID NO: 34:
QVQLVQSGAEVKKPGASVKVSCKVSGYTLTRQSMHWVRQAPGKGLEWMGRINTETGEPTYAQKF
QGRVTMTQDTSTDTAYMELSSLRSEDTAVYFCAIFYYGRDTWGQGTTVTVSS
SEQ ID NO: 45:
EIVMTQSPATLSVSPGERVTLSCKSSQSLLRRGDLATIHGNALAWYQQKPGQAPRLLIYWASTR
ETGVPARFSGSGSGTEFTLTISSLQSEDFAVYYCKQSYNLLSFGQGTVLEIKR
H4.C8 RGD1 K (VH FR2 Q to E mutation); Italic text = Q to E
mutation, E balances charge distribution; Q mutation increases
affinity and expression
SEQ ID NO: 37:
QVQLVQSGAEVKKPGASVKVSCKVSGYTLTRQSMHWVRQAPGKGLEWMGRINTETGEPTYAQKF
QGRVTMTEDTSTDTAYMELSSLRSEDTAVYFCAIFYYGRDTWGQGTTVTVSS
SEQ ID NO: 44:
EIVMTQSPATLSVSPGERVTLSCKSSQSLLRRGDLATIHGNALAWYQQKPGQAPRLLIYWASTR
ETGVPARFSGSGSGTEFTLTISSLQSEDFAVYYCKQSYNLLSFGQGTKVEIKR
H4.C8 RGD1 K (VH FR2 Q to E mutation) Italic text = Q to E
mutation; Q mutation increases affinity and expression, E
balances charge distribution; (KV FR4 to VL mutation) Italic
text = mouse residues in SEQ ID NO: 45
SEQ ID NO: 37:
QVQLVQSGAEVKKPGASVKVSCKVSGYTLTRQSMHWVRQAPGKGLEWMGRINTETGEPTYAQKF
QGRVTMTEDTSTDTAYMELSSLRSEDTAVYFCAIFYYGRDTWGQGTTVTVSS
SEQ ID NO: 45:
EIVMTQSPATLSVSPGERVTLSCKSSQSLLRRGDLATIHGNALAWYQQKPGQAPRLLIYWASTR
ETGVPARFSGSGSGTEFTLTISSLQSEDFAVYYCKQSYNLLSFGQGTVLEIKR
H4.C8 RGD1 R (VL FR4 to KV mutation)
SEQ ID NO: 34
QVQLVQSGAEVKKPGASVKVSCKVSGYTLTRQSMHWVRQAPGKGLEWMGRINTETGEPTYAQKF
QGRVTMTQDTSTDTAYMELSSLRSEDTAVYFCAIFYYGRDTWGQGTTVTVSS
SEQ ID NO: 46:
EIVMTQSPATLSVSPGERVTLSCRSSQSLLRRGDLATIHGNALAWYQQKPGQAPRLLIYWASTR
ETGVPARFSGSGSGTEFTLTISSLQSEDFAVYYCKQSYNLLSFGQGTKVEIKR
H4.C8 RGD1 R (KV FR4 to VL mutation). Italic text = mouse residues
SEQ ID NO: 34:
QVQLVQSGAEVKKPGASVKVSCKVSGYTLTRQSMHWVRQAPGKGLEWMGRINTETGEPTYAQKF
QGRVTMTQDTSTDTAYMELSSLRSEDTAVYFCAIFYYGRDTWGQGTTVTVSS
SEQ ID NO: 47:
EIVMTQSPATLSVSPGERVTLSCRSSQSLLRRGDLATIHGNALAWYQQKPGQAPRLLIYWASTR
ETGVPARFSGSGSGTEFTLTISSLQSEDFAVYYCKQSYNLLSFGQGTVLEIKR
H4.C8 RGD1 R (VH FR2 Q to E mutation) Bold text = Q to E
mutation, E balances charge distribution; Q mutation increases
affinity and expression
SEQ ID NO: 37:
QVQLVQSGAEVKKPGASVKVSCKVSGYTLTRQSMHWVRQAPGKGLEWMGRINTETGEPTYAQKF
QGRVTMTEDTSTDTAYMELSSLRSEDTAVYFCAIFYYGRDTWGQGTTVTVSS
SEQ ID NO: 46:
EIVMTQSPATLSVSPGERVTLSCRSSQSLLRRGDLATIHGNALAWYQQKPGQAPRLLIYWASTR
ETGVPARFSGSGSGTEFTLTISSLQSEDFAVYYCKQSYNLLSFGQGTKVEIKR
H4.C8 RGD1 R (VH FR2 Q to E mutation) Italic text = Q to E
mutation; Q mutation increases affinity and expression, E
balances charge distribution; (KV FR4 to VL mutation) Bold text =
mouse residues in SEQ ID NO: 47
SEQ ID NO: 37:
QVQLVQSGAEVKKPGASVKVSCKVSGYTLTRQSMHWVRQAPGKGLEWMGRINTETGEPTYAQKF
QGRVTMTEDTSTDTAYMELSSLRSEDTAVYFCAIFYYGRDTWGQGTTVTVSS
SEQ ID NO: 47:
EIVMTQSPATLSVSPGERVTLSCRSSQSLLRRGDLATIHGNALAWYQQKPGQAPRLLIYWASTR
ETGVPARFSGSGSGTEFTLTISSLQSEDFAVYYCKQSYNLLSFGQGTVLEIKR
Human TGFB3
>sp|P10600|TGFB3_HUMAN Transforming growth factor beta-3 proprotein
SEQ ID NO: 48
MKMHLQRALVVLALLNFATVSLSLSTCTTLDFGHIKKKRVEAIRGQILSKLRLTSPPEPTVMTH
VPYQVLALYNSTRELLEEMHGEREEGCTQENTESEYYAKEIHKFDMIQGLAEHNELAVCPKGIT
SKVFRFNVSSVEKNRTNLFRAEFRVLRVPNPSSKRNERIELFQILRPDEHIAKQRYIGGKNLPT
RGTAEWLSFDVTDTVREWLLRRESNLGLEISIHCPCHTFQPNGDILENIHEVMEIKFKGVDNED
DHGRGDLGRLKKQKDHHNPHLILMMIPPHRLDNPGQGGQRKKRALDTNYCFRNLEENCCVRPLY
IDFRQDLGWKWVHEPKGYYANFCSGPCPYLRSADTTHSTVLGLYNTLNPEASASPCCVPQDLEP
LTILYYVGRTPKVEQLSNMVVKSCKCS
Human TGFB3 RGD loop
SEQ ID NO: 49 GRGDLGRLKK
Human TGFB1 >sp|P01137|TGFB1_HUMAN Transforming growth factor beta-1
proprotein
SEQ ID NO: 50
MPPSGLRLLLLLLPLLWLLVLTPGRPAAGLSTCKTIDMELVKRKRIEAIRGQILSKLRLASPPS
QGEVPPGPLPEAVLALYNSTRDRVAGESAEPEPEPEADYYAKEVTRVLMVETHNEIYDKFKQST
HSIYMFFNTSELREAVPEPVLLSRAELRLLRLKLKVEQHVELYQKYSNNSWRYLSNRLLAPSDS
PEWLSFDVTGVVRQWLSRGGEIEGFRLSAHCSCDSRDNTLQVDINGFTTGRRGDLATIHGMNRP
FLLLMATPLERAQHLQSSRHRRALDTNYCFSSTEKNCCVRQLYIDFRKDLGWKWIHEPKGYHAN
FCLGPCPYIWSDTQYSKVLALYNQHNPGASAAPCCVPQALEPLPIVYYVGRKPKVEQLSNMIVR
SCKCS
Human TGFB1 RGD loop
SEQ ID NO: 51 RRGDLATIHG
Integrin b8 subunit extracellular domain
SEQ ID NO: 52
MCGSALAFFTAAFVCLQNDRRGPASFLWAAWVFSLVLGLGQGEDNRCASSNAASCARCLALGPE
CGWCVQEDFISGGSRSERCDIVSNLISKGCSVDSIEYPSVHVIIPTENEINTQVTPGEVSIQLR
PGAEANFMLKVHPLKKYPVDLYYLVDVSASMHNNIEKNSVGNDLSRKMAFFSRDFRLGFGSYVD
KTVSPYISIHPERIHNQCSDYNLDCMPPHGYIHVLSLTENITEFEKAVHRQKISGNIDTPEGGF
DAMLQAAVCESHIGWRKEAKRLLLVMTDQTSHLALDSKLAGIVVPNDGNCHLKNNVYVKSTTME
HPSLGQLSEKLIDNNINVIFAVQGKQFHWYKDLLPLLPGTIAGEIESKAANLNNLVVEAYQKLI
SEVKVQVENQVQGIYFNITAICPDGSRKPGMEGCRNVTSNDEVLFNVTVTMKKCDVTGGKNYAI
IKPIGFNETAKIHIHRNCSCQCEDNRGPKGKCVDETFLDSKCFQCDENKCHFDEDQFSSESCKS
HKDQPVCSGRGVCVCGKCSCHKIKLGKVYGKYCEKDDFSCPYHHGNLCAGHGECEAGRCQCFSG
WEGDRCQCPSAAAQHCVNSKGQVCSGRGTCVCGRCECTDPRSIGRFCEHCPTCYTACKENWNCM
QCLHPHNLSQAILDQCKTSCALMEQQHYVDQTSECESSPS
Integrin av subunit extracellular domain
SEQ ID NO: 53
MAFPPRRRLRLGPRGLPLLLSGLLLPLCRAFNLDVDSPAEYSGPEGSYFGFAVDFFVPSASSRM
FLLVGAPKANTTQPGIVEGGQVLKCDWSSTRRCQPIEFDATGNRDYAKDDPLEFKSHQWFGASV
RSKQDKILACAPLYHWRTEMKQEREPVGTCFLQDGTKVEYAPCRSQDIDADGQGFCQGGFSIDF
TKADRVLLGGPGSFYWQGQLISDQVAEIVSKYDPNVYSIKYNNQLATRTAQAIFDDSYLGYSVA
VGDFNGDGIDDFVSGVPRAARTLGMVYIYDGKNMSSLYNFTGEQMAAYFGFSVAATDINGDDYA
DVFIGAPLFMDRGSDGKLQEVGQVSVSLQRASGDFQTTKLNGFEVFARFGSAIAPLGDLDQDGF
NDIAIAAPYGGEDKKGIVYIFNGRSTGLNAVPSQILEGQWAARSMPPSFGYSMKGATDIDKNGY
PDLIVGAFGVDRAILYRARPVITVNAGLEVYPSILNQDNKTCSLPGTALKVSCFNVRFCLKADG
KGVLPRKLNFQVELLLDKLKQKGAIRRALFLYSRSPSHSKNMTISRGGLMQCEELIAYLRDESE
FRDKLTPITIFMEYRLDYRTAADTTGLQPILNQFTPANISRQAHILLDCGEDNVCKPKLEVSVD
SDQKKIYIGDDNPLTLIVKAQNQGEGAYEAELIVSIPLQADFIGVVRNNEALARLSCAFKTENQ
TRQVVCDLGNPMKAGTQLLAGLRFSVHQQSEMDTSVKFDLQIQSSNLFDKVSPVVSHKVDLAVL
AAVEIRGVSSPDHVFLPIPNWEHKENPETEEDVGPVVQHIYELRNNGPSSFSKAMLHLQWPYKY
NNNTLLYILHYDIDGPMNCTSDMEINPLRIKISSLQTTEKNDTVAGQGERDHLITKRDLALSEG
DIHTLGCGVAQCLKIVCQVGRLDRGKSAILYVKSLLWTETFMNKENQNHSYSLKSSASFNVIEF
PYKNLPIEDITNSTLVTTNVTWGIQPAPMPVP
LRRC32 (GARP) >sp|Q14392|LRC32_HUMAN Transforming growth factor
beta activator
SEQ ID NO: 54
MRPQILLLLALLTLGLAAQHQDKVPCKMVDKKVSCQVLGLLQVPSVLPPDTETLDLSGNQLRSI
LASPLGFYTALRHLDLSTNEISFLQPGAFQALTHLEHLSLAHNRLAMATALSAGGLGPLPRVTS
LDLSGNSLYSGLLERLLGEAPSLHTLSLAENSLTRLTRHTFRDMPALEQLDLHSNVLMDIEDGA
FEGLPRLTHLNLSRNSLTCISDFSLQQLRVLDLSCNSIEAFQTASQPQAEFQLTWLDLRENKLL
HFPDLAALPRLIYLNLSNNLIRLPTGPPQDSKGIHAPSEGWSALPLSAPSGNASGRPLSQLLNL
DLSYNEIELIPDSFLEHLTSLCFLNLSRNCLRTFEARRLGSLPCLMLLDLSHNALETLELGARA
LGSLRTLLLQGNALRDLPPYTFANLASLQRLNLQGNRVSPCGGPDEPGPSGCVAFSGITSLRSL
SLVDNEIELLRAGAFLHTPLTELDLSSNPGLEVATGALGGLEASLEVLALQGNGLMVLQVDLPC
FICLKRLNLAENRLSHLPAWTQAVSLEVLDLRNNSFSLLPGSAMGGLETSLRRLYLQGNPLSCC
GNGWLAAQLHQGRVDVDATQDLICRFSSQEEVSLSHVRPEDCEKGGLKNINLIIILTFILVSAI
LLTTLAACCCVRRQKFNQQYKA
HC CDR1
SEQ ID NO: 55: (E/R)QSMH
F12 FR3
SEQ ID NO: 56:
RFTVTLDTSTSTAYLEIRSLRSDDTAVYFCAI
HC CDR3:
SEQ ID NO: 57: FYYGRDS
HC CDR3:
SEQ ID NO: 58: FYYGRDT
Human IgG4 (S228P R409K L445P) CH1-CH3:
SEQ ID NO: 59
ASTKGPSVFPLAPCSRSTSESTAALGCLVKDYFPEPVTVSWNSGALTSGVHTFPAVLQSSGLYS
LSSVVTVPSSSLGTKTYTCNVDHKPSNTKVDKRVESKYGPPCPPCPAPEFLGGPSVFLFPPKPK
DTLMISRTPEVTCVVVDVSQEDPEVQFNWYVDGVEVHNAKTKPREEQFNSTYRVVSVLTVLHQD
WLNGKEYKCKVSNKGLPSSIEKTISKAKGQPREPQVYTLPPSQEEMTKNQVSLTCLVKGFYPSD
IAVEWESNGQPENNYKTTPPVLDSDGSFFLYSKLTVDKSRWQEGNVFSCSVMHEALHNHYTQKS
LSLSPGK
Mouse IgG2a CH1-CH3:
SEQ ID NO: 60:
ASTKGPSVFPLAPSSKSTSGGTAALGCLVKDYFPEPVTVSWNSGALTSGVHTFPAVLQSSGLYS
LSSVVTVPSSSLGTQTYICNVNHKPSNTKVDKKIVPRDCGCKPCICTVPEVSSVFIFPPKPKDV
LTITLTPKVTCVVVDISKDDPEVQFSWFVDDVEVHTAQTQPREEQFNSTFRSVSELPIMHQDWL
NGKEFKCRVNSAAFPAPIEKTISKTKGRPKAPQVYTIPPPKEQMAKDKVSLTCMITDFFPEDIT
VEWQWNGQPAENYKNTQPIMDTDGSYFVYSKLNVQKSNWEAGNTFTCSVLHEGLHNHHTEKSLS
HSPGK
Mouse IgG1 (D265A) CH1-CH3:
SEQ ID NO: 61:
ASTKGPSVFPLAPSSKSTSGGTAALGCLVKDYFPEPVTVSWNSGALTSGVHTFPAVLQSSGLYS
LSSVVTVPSSSLGTQTYICNVNHKPSNTKVDKKIVPRDCGCKPCICTVPEVSSVFIFPPKPKDV
LTITLTPKVTCVVVAISKDDPEVQFSWFVDDVEVHTAQTQPREEQFNSTFRSVSELPIMHQDWL
NGKEFKCRVNSAAFPAPIEKTISKTKGRPKAPQVYTIPPPKEQMAKDKVSLTCMITDFFPEDIT
VEWQWNGQPAENYKNTQPIMDTDGSYFVYSKLNVQKSNWEAGNTFTCSVLHEGLHNHHTEKSLS
HSPGK
ADWA 11 2.4 Vh (SEQ ID NO: 2 from U.S. 2020/0079855 A1)
SEQ ID NO: 62:
EVQLVESGGGLVQPGGSLRLSCAASGFNIKDYYMNWVRQAPGKGLEWVGWIDPDQGNTIYEPKF
QGRFTISADTSKNSAYLQMNSLRAEDTAVYYCARRLLMDYWGQGTLVTVSS
ADWA 11 2.4 light chain (SEQ ID NO: 5 U.S. 2020/0079855 A1)
SEQ ID NO: 63:
DIQMTQSPSSLSASVGDRVTITCRSTKSLSHENGNTYLFWYQQKPGKAPKRLIYYMSSLASGVP
SRFSGSGSGTDFTLTISSLQPEDFATYYCQQSLEYPFTFGGGTKVEIKR
SRK-181 (Ab6) Vh (SEQ ID NO: 13 from patent WO 2020/014460 A1)
SEQ ID NO: 64:
EVQLVESGGGLVQPGGSLRLSCTASGFTFSSFSMDWVRQAPGKGLEWVSYISPSADTIYYAD
SVKGRFTISRDNAKNTLYLQMNSLRAEDTAVYYCARGVLDYGDMLMPWGQGTLVTVSS
SRK-181 (Ab6) light chain (SEQ ID NO: 15 from patent WO 2020/014460 A1)
SEQ ID NO: 65:
DIQMTQSPSSLSASVGDRVTITCQASQDITNYLNWYQQKPGKAPKLLIYDASNLETGVPSRFSG
SGSGTDFTFTISSLQPEDIATYYCQQADNHPPWTFGGGTKVEIKR
H3.12 VH
SEQ ID NO: 66:
QVQLVQSGAEVKKPGASVKVSCKVSGYTLTEQSMHWVRQAPGKGLEWMGRINTETGEPTYAQKF
QGRVTMTQDTSTDTAYMELSSLRSEDTAVYFCAIFYYGRDTWGQGTTVTVSS
H3.12 VL
SEQ ID NO: 67:
EIVMTQSPATLSVSPGERVTMSCRSSQSLLHSRTRKNYLAWYQQKPGQAPRLLIYWASTRETGV
PARFSGSGSGTEFTLTISSLQSEDFAVYYCKQSYNLLSFGQGTVLEIKR
B4 VH
SEQ ID NO: 68:
QVQLVQSGAEVKKPGASVKVSCKVSGYTLTKLSMHWVRQAPGKGLEWMGRINPEAGEPTYAQKF
QGRVTMTVDTSTDTAYMELSSLRSEDTAVYYCAIFYYGRDSWGQGTTLTVSS
B4 VL
SEQ ID NO: 69:
EIVMTQSPATLSVSPGERATLSCRASQSLLHSRTRKNYLAWYQQKPGQAPRLLIYWASTRATGI
PARFSGSGSGTEFTLTISSLQSEDFAVYYCKQSYNLLSFGQGTVLEIKR
H1.1 VH
SEQ ID NO: 70:
QVQLVQSGAEVKKPGASVKVSCKVSGYTLTKLSMHWVRQAPGKGLEWMGRINTEDGEPIYAQKF
QGRVTMTEDTSTDTAYMELSSLRSEDTAVYYCAIFYYGRDSWGQGTTLTVSS
H1.1 VK
SEQ ID NO: 71:
EIVMTQSPATLSVSPGERATLSCRASQSLLHSRTRKNYLAWYQQKPGQAPRLLIYWASTRATGI
PARFSGSGSGTEFTLTISSLQSEDFAVYYCKQSYNLLSFGQGTVLEIKR
A5 VH
SEQ ID NO: 72:
QVQLVQSGAEVKKPGASVKVSCKVSGYTLTRHSMHWVRQAPGKGLEWMGRINPEAGEPTYAQKF
QGRVTMTQDTSTDTAYLELSSLRSEDTAVYYCAIFYYGRDSWGQGTTLTVSS
A10 VH
SEQ ID NO: 73:
QVQLVQSGAEVKKPGASVKVSCKVSGYTLTRQSMHWVRQAPGKGLEWMGRINTETGEPTYAQKF
QGRVTMTEDTSTDTAYMELSSLRSEDTAVYYCAIFYYGRDSWGQGTTLTVSS
A10 VL
SEQ ID NO: 74:
EIVMTQSPATLSVSPGERATLSCRSSQSLLHSRTRKNYLAWYQQKPGQAPRLLIYWASTRDTGI
PARFSGSGSGTEFTLTISSLQSEDFAVYYCKQSYNLLSFGQGTVLEIKR
A12 VH
SEQ ID NO: 75:
QVQLVQSGAEVKKPGASVKVSCKVSGYTLTEQSMHWVRQAPGKGLEWMGRINTETGEPTYAQKF
QGRVTMTQDTSTDTAYMELSSLRSEDTAVYYCAIFYYGRDSWGQGTTLTVSS
A12 VL
SEQ ID NO: 76:
EIVMTQSPATLSVSPGERATLSCRSSQSLLHSRTRKNYLAWYQQKPGQAPRLLIYWASTRATGI
PARFSGSGSGTEFTLTISSLQSEDFAVYYCKQSYNLLSFGQGTVLEIKR
C12 VH
SEQ ID NO: 77:
QVQLVQSGAEVKKPGASVKVSCKVSGYTLTEQSMHWVRQAPGKGLEWMGRINTENGEPTYAQKF
QGRVTMTQDTSTDTAYLELSSLRSEDTAVYYCAIFYYGRDSWGQGTTLTVSS
C12 VL
SEQ ID NO: 78:
EIVMTQSPATLSVSPGERATLSCRSSQSLLHSRTRKNYLAWYQQKPGQAPRLLIYWASTRATGI
PARFSGSGSGTEFTLTISSLQSEDFAVYYCKQSYNLLSFGQGTVLEIKR
HuF12 Fr1 human VH
SEQ ID NO: 79:
QVQLVQSGAEVKKPGASVKVSCKVSGYTFTKYSMHWVRQAPGQGLEWVARINTETGEPTFADDF
RGRFTVTLDTSTSTAYLEIRSLRSDDTAVYFCAIFYYGRDTWGQGTTLTVSS
B4 Fr1/CDR1/F12 VH
SEQ ID NO: 80:
QVQLVQSGAEVKKPGASVKVSCKVSGYTFTKLSMHWVRQAPGQGLEWVARINTETGEPTFADDF
RGRFTVTLDTSTSTAYLEIRSLRSDDTAVYFCAIFYYGRDTWGQGTTLTVSS
A5 Fr1/CDR1/F12 VH
SEQ ID NO: 81:
QVQLVQSGAEVKKPGASVKVSCKVSGYTFTRHSMHWVRQAPGQGLEWVARINTETGEPTFADDF
RGRFTVTLDTSTSTAYLEIRSLRSDDTAVYFCAIFYYGRDTWGQGTTLTVSS
A10 Fr1/CDR1/F12 VH
SEQ ID NO: 82:
QVQLVQSGAEVKKPGASVKVSCKVSGYTFTKLSMHWVRQAPGQGLEWVARINTETGEPTFADDF
RGRFTVTLDTSTSTAYLEIRSLRSDDTAVYFCAIFYYGRDTWGQGTTLTVSS
A12 Fr1/CDR1/F12 VH
SEQ ID NO: 83:
QVQLVQSGAEVKKPGASVKVSCKVSGYTFTEQSMHWVRQAPGQGLEWVARINTETGEPTFADDF
RGRFTVTLDTSTSTAYLEIRSLRSDDTAVYFCAIFYYGRDTWGQGTTLTVSS
MHG-8 VH (from patent EP2832747A1)
SEQ ID NO: 84:
QVQLKESGPGLVAPSQSLSITCTVSGFSLTGYGINWVRQPPGKGLEWLGMIWSDGSTDYNSVLT
SRLRISKDNSNSQVFLKMNSLQVDDTARYYCARDRNYYDYDGAMDYWGQGTSVTVSS
MHG-8 VL (from patent EP2832747A1)
SEQ ID NO: 85:
DIQVTQSSSYLSVSLGDRVTITCKASDHIKNWLAWYQQKPGIAPRLLVSGATSLEAGVPSRFSG
SGSGKNFTLSITSLQTEDVATYYCQQYWSTPWTFGGGTTLEIR
Mouse IgG1 CH1-CH3:
SEQ ID NO: 86:
ASTKGPSVFPLAPSSKSTSGGTAALGCLVKDYFPEPVTVSWNSGALTSGVHTFPAVLQSSGLYS
LSSVVTVPSSSLGTQTYICNVNHKPSNTKVDKKIVPRDCGCKPCICTVPEVSSVFIFPPKPKDV
LTITLTPKVTCVVVDISKDDPEVQFSWFVDDVEVHTAQTQPREEQFNSTFRSVSELPIMHQDWL
NGKEFKCRVNSAAFPAPIEKTISKTKGRPKAPQVYTIPPPKEQMAKDKVSLTCMITDFFPEDIT
VEWQWNGQPAENYKNTQPIMDTDGSYFVYSKLNVQKSNWEAGNTFTCSVLHEGLHNHHTEKSLS
HSPGK
F12 scFV
SEQ ID NO: 87
QIQLVQSGAEVKKPGASVKISCKASGYTFTKYSMHWVRQAPGQGLEWVARINTETGEPTFADDF
RGRFTVILDTSTSTAYLEIRSLRSDDTAVYFCAIFYYGRDTWGQGTTLTVSSVDSKSGGSTSGS
GKPGSGEGSSGSAREIVMTQSPATLSVSPGERVTMSCKSSQSLLHSRSRKNYLAWYQQKPGQAP
RLLIYWASTRESGVPARFSGSGSGTEFTLTISSVQSEDFAVYYCKQSYNLLSFGQGTVLEIKR
H4C8 scFV
SEQ ID NO: 88
QVQLVQSGAEVKKPGASVKVSCKVSGYTLTRQSMHWVRQAPGKGLEWMGRINTETGEPTYAQKF
QGRVTMTQDTSTDTAYMELSSLRSEDTAVYFCAIFYYGRDTWGQGTTVTVSSVDSKSGGSTSGS
GKPGSGEGSSGSAREIVMTQSPATLSVSPGERVTLSCRSSQSLLHSRSRKNYLAWYQQKPGQAP
RLLIYWASTRETGVPARFSGSGSGTEFTLTISSLQSEDFAVYYCKQSYNLLSFGQGTVLEIKR
Murine CD28/CD3epsilon
SEQ ID NO: 89
IEFMYPPPYLDNERSNGTIIHIKEKHLCHTQSSPKLFWALVVVAGVLFCYGLLVTVALCVIWTN
SRRNRGGQSDYMNMTPRRPGLTRKPYQPYAPARDFAAYRPRAKFSRSAETAANLQDPNQLFNEL
NLGRREEFDVLEKKRARDPEMGGKQQRRRNPQEGVYNALQKDKMAEAYSEIGTKGERRRGKGHD
GLFQGLSTATKDTFDALHMQTLAPR
F12/CD28/CD3epsilon
SEQ ID NO: 90:
QIQLVQSGAEVKKPGASVKISCKASGYTFTKYSMHWVRQAPGQGLEWVARINTETGEPTFADDF
RGRFTVTLDTSTSTAYLEIRSLRSDDTAVYFCAIFYYGRDTWGQGTTLTVSSVDSKSGGSTSGS
GKPGSGEGSSGSAREIVMTQSPATLSVSPGERVTMSCKSSQSLLHSRSRKNYLAWYQQKPGQAP
RLLIYWASTRESGVPARFSGSGSGTEFTLTISSVQSEDFAVYYCKQSYNLLSFGQGTVLEIKRI
EFMYPPPYLDNERSNGTIIHIKEKHLCHTQSSPKLFWALVVVAGVLFCYGLLVTVALCVIWTNS
RRNRGGQSDYMNMTPRRPGLTRKPYQPYAPARDFAAYRPRAKFSRSAETAANLQDPNQLFNELN
LGRREEFDVLEKKRARDPEMGGKQQRRRNPQEGVYNALQKDKMAEAYSEIGTKGERRRGKGHDG
LFQGLSTATKDTFDALHMQTLAPR
H4C8/CD28/CD3epsilon
SEQ ID NO: 91:
QVQLVQSGAEVKKPGASVKVSCKVSGYTLTRQSMHWVRQAPGKGLEWMGRINTETGEPTYAQKF
QGRVTMTQDTSTDTAYMELSSLRSEDTAVYFCAIFYYGRDTWGQGTTVTVSSVDSKSGGSTSGS
GKPGSGEGSSGSAREIVMTQSPATLSVSPGERVTLSCRSSQSLLHSRSRKNYLAWYQQKPGQAP
RLLIYWASTRETGVPARFSGSGSGTEFTLTISSLQSEDFAVYYCKQSYNLLSFGQGTVLEIKRI
EFMYPPPYLDNERSNGTIIHIKEKHLCHTQSSPKLFWALVVVAGVLFCYGLLVTVALCVIWTNS
RRNRGGQSDYMNMTPRRPGLTRKPYQPYAPARDFAAYRPRAKFSRSAETAANLQDPNQLFNELN
LGRREEFDVLEKKRARDPEMGGKQQRRRNPQEGVYNALQKDKMAEAYSEIGTKGERRRGKGHDG
LFQGLSTATKDTFDALHMQTLAPR

### Tables Showing Exemplary CDR and Variable Region Pairings

TABLE VIII

| CDRS occur | | | Kabat | | |
|---|---|---|---|---|---|
| in Seq ID(s) | Clone | Chain | CDR1 | CDR2 | CDR3 |
| #34, #37 | H4 | HC | RQSMH (SEQ ID NO: 1) | RINTETGEPTYAQKFQG (SEQ ID NO: 2) | FYYGRD (S/T) (SEQ ID NO: 3) |
| #35, #36 | C8 | LC | RSSQSLLHSRSRKNYLA (SEQ ID NO: 4) | WASTRET (SEQ ID NO: 5) | KQSYNLLS (SEQ ID NO: 6) |
| #38, #39 | H3 | HC | EQSMH (SEQ ID NO: 7) | RINTETGEPTYAQKFQG (SEQ ID NO: 2) | FYYGRD (S/T) (SEQ ID NO: 3) |
| #35, #36 | C8 | LC | RSSQSLLHSRSRKNYLA (SEQ ID NO: 4) | WASTRET (SEQ ID NO: 5) | KQSYNLLS (SEQ ID NO: 6) |
| #34, #37 | H4 | HC | RQSMH (SEQ ID NO: 1) | RINTETGEPTYAQKFQG (SEQ ID NO: 2) | FYYGRDT (SEQ ID NO: 3) |
| #40, #41 | RGD3 K | LC | KSSQSLLGRGDLGRLKKNALA (SEQ ID NO: 8) | WASTRET (SEQ ID NO: 5) | KQSYNLLS (SEQ ID NO: 6) |
| #34, #37 | H4 | HC | RQSMH (SEQ ID NO: 1) | RINTETGEPTYAQKFQG (SEQ ID NO: 2) | FYYGRD (S/T) (SEQ ID NO: 3) |
| #42, #43 | RGD3 R | LC | RSSQSLLGRGDLGRLKKNALA (SEQ ID NO: 9) | WASTRET (SEQ ID NO: 5) | KQSYNLLS (SEQ ID NO: 6) |
| #38, #39 | H3 | HC | EQSMH (SEQ ID NO: 7) | RINTETGEPTYAQKFQG (SEQ ID NO: 2) | FYYGRD (S/T) (SEQ ID NO: 3) |
| #40, #41 | RGD3 K | LC | KSSQSLLGRGDLGRLKKNALA (SEQ ID NO: 8) | WASTRET (SEQ ID NO: 5) | KQSYNLLS (SEQ ID NO: 6) |
| #38, #39 | H3 | HC | EQSMH (SEQ ID NO: 7) | RINTETGEPTYAQKFQG (SEQ ID NO: 2) | FYYGRD (S/T) (SEQ ID NO: 3) |
| #42, #43 | RGD3 R | LC | RSSQSLLRRGDLATIHGNALA (SEQ ID NO: 9) | WASTRET (SEQ ID NO: 5) | KQSYNLLS (SEQ ID NO: 6) |
| #34, #37 | H4 | HC | RQSMH (SEQ ID NO: 1) | RINTETGEPTYAQKFQG (SEQ ID NO: 2) | FYYGRD (S/T) (SEQ ID NO: 3) |
| #44, #45 | RGD1 K | LC | KSSQSLLRRGDLATIHGNALA (SEQ ID NO: 10) | WASTRET (SEQ ID NO: 5) | KQSYNLLS (SEQ ID NO: 6) |
| #34, #37 | H4 | HC | RQSMH (SEQ ID NO: 1) | RINTETGEPTYAQKFQG (SEQ ID NO: 2) | FYYGRD (S/T) (SEQ ID NO: 3) |
| #46, #47 | RGD1 R | LC | RSSQSLLRRGDLATIHGNALA (SEQ ID NO: 11) | WASTRET (SEQ ID NO: 5) | KQSYNLLS (SEQ ID NO: 6) |
| #38, #39 | H3 | HC | EQSMH (SEQ ID NO: 7) | RINTETGEPTYAQKFQG (SEQ ID NO: 2) | FYYGRD (S/T) (SEQ ID NO: 3) |
| #44, #45 | RGD1 K | LC | KSQSLLRRGDLATIHGNALA (SEQ ID NO: 10) | WASTRET (SEQ ID NO: 5) | KQSYNLLS (SEQ ID NO: 6) |
| #38, #39 | H3 | HC | EQSMH (SEQ ID NO: 7) | RINTETGEPTYAQKFQG (SEQ ID NO: 2) | FYYGRD (S/T) (SEQ ID NO: 3) |
| #46, #47 | RGD1 R | LC | RSSQSLLRRGDLATIHGNALA (SEQ ID NO: 11) | WASTRET (SEQ ID NO: 5) | KQSYNLLS (SEQ ID NO: 6) |

TABLE IX

| FRs occur | | Kabat | | | |
|---|---|---|---|---|---|
| in Seq ID(s) | Chain | Fr1 | Fr2 | Fr3 | Fr4 |
| #34, #38, | HC | QVQLVQSGAEVKKPGASVKVSCKVSGYTLT | WVRQAPGKGLEWMG | RVTMTQDTSTDTAYMELSSLRSEDTAVYFCAI | WGQGTTVTVSS |
| | | (SEQ ID NO: 12) | (SEQ ID NO: 13) | (SEQ ID NO: 14) | (SEQ ID |
| | | | | | NO: 15) |
| #35, #40, | LC | EIVMTQSPATLSVSPGERVTLSC | WYQQKPGQAPRLLIY | GVPARFSGSGSGTEFTLTISSLQSEDFAVYYC | FGQGTKVEIK |
| #42 | | (SEQ ID NO: 16) | (SEQ ID NO: 17) | (SEQ ID NO: 18) | (SEQ ID |
| #44, #46 | | | | | NO: 19) |
| #34, #38 | HC | QVQLVQSGAEVKKPGASVKVSCKVSGYTLT | WVRQAPGKGLEWMG | RVTMTQDTSTDTAYMELSSLRSEDTAVYFCAI | WGQGTTVTVSS |
| | | (SEQ ID NO: 12) | (SEQ ID NO: 13) | (SEQ ID NO: 14) | (SEQ ID |
| | | | | | NO: 15) |
| #36, #41, | LC | EIVMTQSPATLSVSPGERVTLSC | WYQQKPGQAPRLLIY | GVPARFSGSGSGTEFTLTISSLQSEDFAVYYC | FGQGTVLEIK |
| #43 | | (SEQ ID NO: 16) | (SEQ ID NO: 17) | (SEQ ID NO: 18) | (SEQ ID |
| #45, #47 | | | | | NO: 20) |
| #37, #39 | HC | QVQLVQSGAEVKKPGASVKVSCKVSGYTLT | WVRQAPGKGLEWMG | RVTMTEDTSTDTAYMELSSLRSEDTAVYFCAI | WGQGTTVTVSS |
| | | (SEQ ID NO: 12) | (SEQ ID NO: 13) | (SEQ ID NO: 21) | (SEQ ID |
| | | | | | NO: 15) |
| #35, #40, | LC | EIVMTQSPATLSVSPGERVTLSC | WYQQKPGQAPRLLIY | GVPARFSGSGSGTEFTLTISSLQSEDFAVYYC | FGQGTKVEIK |
| #42 | | (SEQ ID NO: 16) | (SEQ ID NO: 17) | (SEQ ID NO: 18) | (SEQ ID |
| #44, #46 | | | | | NO: 19) |
| #37, #39 | HC | QVQLVQSGAEVKKPGASVKVSCKVSGYTLT | WVRQAPGKGLEWMG | RVTMTEDTSTDTAYMELSSLRSEDTAVYFCAI | WGQGTTVTVSS |
| | | (SEQ ID NO: 12) | (SEQ ID NO: 13) | (SEQ ID NO: 21) | (SEQ ID |
| | | | | | NO: 15) |
| #34, #41, | LC | EIVMTQSPATLSVSPGERVTLSC | WYQQKPGQAPRLLIY | GVPARFSGSGSGTEFTLTISSLQSEDFAVYYC | FGQGTVLEIK |
| #43 | | (SEQ ID NO: 16) | (SEQ ID NO: 17) | (SEQ ID NO: 18) | (SEQ ID |
| #45, #47 | | | | | NO: 20) |

TABLE I
Model refinement and statistics for
the cryo-EM of ADWA11 Fab and avb8

| Symmetry Imposed | C1 |
|---|---|
| Particle used | 46,961 |
| Map resolution (Å) | 4.11 |
| FSC threshold | 0.143 |
| Refinement | |
| Map sharpening B-factor (Å²) | 93.2 |
| All-atom clashscore | 25.67 |
| Ramachandran plot: | |
| outliers: | 0.36% |
| allowed: | 9.83% |
| favored: | 89.81% |
| Rotamer outliers: | 6.74% |

TABLE II
Model refinement and statistics for
the cryo-EM of F12 Fab and avb8

| Symmetry Imposed | C1 |
|---|---|
| Particle used | 160,271 |
| Map resolution (Å) | 3.12 |
| FSC threshold | 0.143 |
| Refinement | |
| Map sharpening B-factor (Å²) | 107.4 |
| All-atom clashscore | 2.22 |
| Ramachandran plot: | |
| outliers: | 0.26% |
| allowed: | 4.44% |
| favored: | 95.30% |
| Rotamer outliers: | 1.39% |

TABLE III
H-bonds

| Interactions | F12 Vl | Dist. [Å] | αν |
|---|---|---|---|
| 1 | SER 32[OG] | 2.57 | ASP 150[OD2] |
| 2 | ARG 33[NH1] | 3.14 | GLU 121[OE2] |
| 3 | ARG 33[NH2] | 2.88 | GLU 121[OE2] |
| 4 | ARG 33[NH2] | 3.48 | GLU 123[OE2] |
| 5 | ARG 35[NH1] | 2.79 | ASP 218[OD2] |
| 6 | ARG 35[NH2] | 3.06 | ASP 218[OD2] |
| 7 | SER 32[O] | 3.76 | ASP 150[N] |
| 8 | SER 32[O] | 2.86 | TYR 178[OH] |
| Interactions | F12 Vl | Dist. [Å] | β8 |
| 1 | LYS 38[N2] | 2.71 | SER 118[OG] |
| 2 | TYR 38[OH] | 3.26 | SER 170[OG] |
| 3 | TRP 58[NE1] | 2.96 | ASP 171[OD2] |
| 4 | LYS 85[N2] | 2.84 | ASN 173[OD1] |
| 5 | SER 87[OG] | 2.84 | ASN 173[ND2] |

Salt Bridges

| Interactions | F12 Vl | Dist. [Å] | αν |
|---|---|---|---|
| 1 | ARG 33[NH1] | 3.14 | GLU 121[OE2] |
| 2 | ARG 33[NH2] | 2.88 | GLU 121[OE2] |
| 3 | ARG 33[NH2] | 3.48 | GLU 123[OE2] |
| 4 | ARG 35[NH1] | 2.78 | ASP 218[OD2] |
| 5 | ARG 35[NH2] | 3.06 | ASP 218[OD2] |
| Interactions | F12 Vh | Dist. [Å] | β8 |

H-bonds

| | | | |
|---|---|---|---|
| 1 | TYR 101[OH] | 3.39 | ASP 171[OD1] |
| 2 | TYR 101[OH] | 3.23 | ASP 171[OD2] |
| 3 | ARG [symbol] [NH1] | 2.89 | TYR 172[O] |
| 4 | SER 33[OG] | 3.02 | ASP 175[OD2] |
| 5 | LYS 31[N [symbol] ] | 2.36 | HIS 200[O] |
| 6 | GLU 54[OE2] | 2.92 | LYS 200[N2] |
| 7 | TYR 101[OH] | 2.35 | ASN 173[ND2] |

Salt Bridges

| | | | |
|---|---|---|---|
| 1 | GLU 54[OE2] | 2.82 | LYS 203[N [symbol] ] |

[symbol]

indicates data missing or illegible when filed

TABLE IV
Clone H1.1 humanized prototype designed using combinatorial structural
analysis incorporating framework-homology-based humanization,
complementary determining regions (CDR)-homology-based humanization,
specificity determining residues (SDR) grafting, and surface veneering
using the F12-avb8 cryoEM structure.
Clone H1.1 as IgG4 has 70-fold lower affinity than F12 mouse IgG2a
Library-1 of backmutations to F12 residues made to isolate clones with
improved affinity compared to H1.1.
New Vh and Vl sequences paired with F12 Vl and Vh sequences respectively
and expressed as scFV, or chimeras made with portions of new clone Vh domains
with F12 Vh domains (Library-2) to identify F12 humanization liabilities
i) Humanization of F12 Vh Fr1 greatly reduces affinity of F12 WT
ii) Humanization of F12 Vl CDR2 (S to A) greatly reduces affinity of F12 WT
Library-3.0 fine tunes mutations back to F12 to identify optimal Vh
CDR1 residues within humanized frameworks
Progeny from Library 3.0 show enrichment of preferred Vh and Vl sequences
and identify sequences with optimal affinity.
Clone H4C8 identified with optimal properties. Clone H4C8 replaces the
F12 CDR1 Vh KYSMH with RQSMH.
New clones converted to IgG4.
Combinations of H4C8 Vh IgG4 and Vl with Vh IgG4 from other clones identify
multiple humanized clones expressed as human IgG4, which maintain high affinity.
Final IgG4 conversion made with additional mutations to humanize Vl Fr4.
H4C8 Vl used to graft integrin binding motif loops of TGF-b1 (RGD1) and
TGF-b3 (RGD3) into Vl CDR1.
New H4C8 Vl paired with H4C8 Vh or H3.C12 Vh (or derivatives) as IgG4
and paired with H4C8 RGD1 and H4C8 RGD3 Vl.
H4C8 RGD3 IgG4 has improved function and binding properties compared
to parental HuC6D4 RGD3.

TABLE V

| | | | | Heavy | Heavy | Heavy | | Heavy | Light | Light | Light | | Light |
|---|---|---|---|---|---|---|---|---|---|---|---|---|---|
| | | OASis | | V | J | OASis | Heavy | Non- | V | J | OASis | Light | Non- |
| | | Per- | OASis | Germ- | Germ- | Per- | OASis | human | Germ- | Germ- | Per- | OASis | human |
| Antibody | Threshold | centile | Identity | line | line | centile | Identity | peptides | line | line | centile | Identity | peptides |
| F12 | relaxed | 0.133 | 0.660 | IGHV1- | IGHJ4*01 | 0.095 | 0.574 | 46 | IGKV3- | IGKJ2*03 | 0.298 | 0.750 | 26 |
| | | | | 2*06 | | | | | 15*01 | | | | |
| H4C8 | relaxed | 0.480 | 0.778 | IGHV1- | IGHJ4*01 | 0.372 | 0.722 | 30 | IGKV3- | IGKJ1*01 | 0.550 | 0.836 | 17 |
| | | | | 24*01 | | | | | 15*01 | | | | |

TABLE VI
H-bonds

| Interactions | [symbol] | Dist. [Å] | β8 |
|---|---|---|---|
| 1 | [symbol] | [symbol] | [symbol] |
| 2 | [symbol] | [symbol] | [symbol] |
| 3 | [symbol] | [symbol] | [symbol] |
| 4 | [symbol] | [symbol] | [symbol] |
| 5 | [symbol] | [symbol] | [symbol] |
| 6 | [symbol] | [symbol] | [symbol] |
| 7 | [symbol] | [symbol] | [symbol] |
| 8 | [symbol] | [symbol] | [symbol] |
| 9 | [symbol] | [symbol] | [symbol] |
| 10 | [symbol] | [symbol] | [symbol] |
| Interactions | [symbol] | Dist. [Å] | αν |
| 1 | [symbol] | [symbol] | [symbol] |
| 2 | [symbol] | [symbol] | [symbol] |
| 3 | [symbol] | [symbol] | [symbol] |
| 4 | [symbol] | [symbol] | [symbol] |
| [symbol] | [symbol] | [symbol] | [symbol] |
| [symbol] | [symbol] | [symbol] | [symbol] |

Salt Bridges

| Interactions | [symbol] Vl | Dist. [Å] | αν |
|---|---|---|---|
| 1 | ARG [symbol] | [symbol] | [symbol] |
| 2 | ARG [symbol] | [symbol] | [symbol] |
| 3 | ARG [symbol] | [symbol] | [symbol] |
| 4 | ARG [symbol] | [symbol] | [symbol] |

| Interactions | [symbol] Vl | Dist. [Å] | β8 |
|---|---|---|---|
| 1 | [symbol] | [symbol] | [symbol] |

H-bonds

| Interactions | [symbol] Vh | Dist. [Å] | [symbol] |
|---|---|---|---|
| 1 | [symbol] | [symbol] | [symbol] |
| 2 | [symbol] | [symbol] | [symbol] |
| 3 | [symbol] | [symbol] | [symbol] |
| 4 | [symbol] | [symbol] | [symbol] |
| 5 | [symbol] | [symbol] | [symbol] |
| 6 | [symbol] | [symbol] | [symbol] |
| 7 | [symbol] | [symbol] | [symbol] |
| 8 | [symbol] | [symbol] | [symbol] |
| 9 | [symbol] | [symbol] | [symbol] |
| 10 | [symbol] | [symbol] | [symbol] |
| 11 | [symbol] | [symbol] | [symbol] |
| 12 | [symbol] | [symbol] | [symbol] |

Salt Bridges

| Interactions | [symbol] Vh | Dist. [Å] | [symbol] |
|---|---|---|---|
| 1 | [symbol] | [symbol] | [symbol] |
| 2 | [symbol] | [symbol] | [symbol] |

[symbol]

indicates data missing or illegible when filed

TABLE VII
Model refinement and statistics for
the cryo-EM of H4C8 Fab and avb8

| Symmetry Imposed | C1 |
|---|---|
| Particle used | 106,895 |
| Map resolution (Å) | 3.19 |
| FSC threshold | 0.143 |
| Refinement | |
| Map sharpening B-factor (Å²) | 101.0 |
| All-atom clashscore | 2.5 |
| Ramachandran plot: | |
| outliers: | 0.60% |
| allowed: | 7.01% |
| favored: | 92.39% |
| Rotamer outliers: | 1.28% |

1. An antibody that specifically binds human integrin αvβ8, wherein the antibody comprises:
(a) a heavy chain variable region comprising a heavy chain complementary determining region (HCDR) 1 comprising RQSMH (SEQ ID NO:1) or EQSMH (SEQ ID NO:7);
a HCDR 2 comprising RINTETGEPTYAQKFQG (SEQ ID NO:2);
a HCDR 3 comprising FYYGRD (S/T) (SEQ ID NO:3); and
(b) a light chain variable region comprising a light chain complementary determining region (LCDR) 1 comprising RSSQSLLHSRSRKNYLA (SEQ ID NO:4)
a LCDR2 comprising WASTRET (SEQ ID NO:5); and
a LCDR3 comprising KQSYNLLS (SEQ ID NO:6).

2. The antibody of claim 1, wherein
the heavy chain variable region comprises: SEQ ID NO:34; and
the light chain variable region comprises: SEQ ID NO:35; or
the heavy chain variable region comprises: SEQ ID NO:34; and
the light chain variable region comprises: SEQ ID NO:36; or
the heavy chain variable region comprises: SEQ ID NO:37; and
the light chain variable region comprises: SEQ ID NO:35; or
the heavy chain variable region comprises: SEQ ID NO:37; and
the light chain variable region comprises: SEQ ID NO:36; or
the heavy chain variable region comprises: SEQ ID NO:38; and
the light chain variable region comprises: SEQ ID NO:35; or
the heavy chain variable region comprises: SEQ ID NO:38; and
the light chain variable region comprises: SEQ ID NO:36; or
the heavy chain variable region comprises: SEQ ID NO:39; and
the light chain variable region comprises: SEQ ID NO:35; or
the heavy chain variable region comprises: SEQ ID NO:39; and
the light chain variable region comprises: SEQ ID NO:36.

3. The antibody of claim 1, wherein the antibody is humanized.

4. The antibody of any one of claims 1-3, wherein the isotype of the antibody is IgG1, IgG2, IgG3 or IgG4.

5. The antibody of any one of claims 1-4, wherein the antibody comprises a constant (Fc) domain and the Fc comprises at least one mutation that reduces effector function, and/or decreases aggregation and/or increases stability.

6. The antibody of any of claims 1-2, wherein the antibody is linked to a detectable label.

7. The antibody of any one of claims 1-6, wherein the antibody is a tetrameric antibody, a Fab, or scfv.

8. An antibody that specifically binds human αvβ8, wherein the antibody comprises:
(a) a heavy chain variable region comprising a heavy chain complementary determining region (HCDR) 1 comprising SEQ ID NO:1 or SEQ ID NO:7;
a HCDR 2 comprising SEQ ID NO:2;
a HCDR 3 comprising SEQ ID NO:3; and
(b) a light chain variable region comprising a light chain complementary determining region (LCDR) 1 comprising KSSQSLLGRGDLGRLKKNALA (SEQ ID NO:8) or RSSQSLLRRGDLATIHGNALA (SEQ ID NO:9) or KSSQSLLRRGDLATIHGNALA (SEQ ID NO: 10) or RSSQSLLRRGDLATIHGNALA (SEQ ID NO:11);
a LCDR2 comprising SEQ ID NO:5; and
a LCDR3 comprising SEQ ID NO:6.

9. The antibody of claim 8, wherein
the heavy chain variable region comprises: SEQ ID NO:34; and
the light chain variable region comprises: SEQ ID NO:40; or
the heavy chain variable region comprises: SEQ ID NO:34; and
the light chain variable region comprises: SEQ ID NO:41; or
the heavy chain variable region comprises: SEQ ID NO:37; and
the light chain variable region comprises: SEQ ID NO:40; or
the heavy chain variable region comprises: SEQ ID NO:37; and
the light chain variable region comprises: SEQ ID NO:41; or
the heavy chain variable region comprises: SEQ ID NO:34; and
the light chain variable region comprises: SEQ ID NO:42; or
the heavy chain variable region comprises: SEQ ID NO:34; and
the light chain variable region comprises: SEQ ID NO:43; or
the heavy chain variable region comprises: SEQ ID NO:37; and
the light chain variable region comprises: SEQ ID NO:42; or
the heavy chain variable region comprises: SEQ ID NO:37; and
the light chain variable region comprises: SEQ ID NO:43; or
the heavy chain variable region comprises: SEQ ID NO:38; and
the light chain variable region comprises: SEQ ID NO:40; or
the heavy chain variable region comprises: SEQ ID NO:38; and
the light chain variable region comprises: SEQ ID NO:41; or
the heavy chain variable region comprises: SEQ ID NO:39; and
the light chain variable region comprises: SEQ ID NO:40; or
the heavy chain variable region comprises: SEQ ID NO:39; and
the light chain variable region comprises: SEQ ID NO:41; or
the heavy chain variable region comprises: SEQ ID NO:38; and
the light chain variable region comprises: SEQ ID NO:42; or
the heavy chain variable region comprises: SEQ ID NO:38; and
the light chain variable region comprises: SEQ ID NO:43; or
the heavy chain variable region comprises: SEQ ID NO:39; and
the light chain variable region comprises: SEQ ID NO:42; or
the heavy chain variable region comprises: SEQ ID NO:39; and
the light chain variable region comprises: SEQ ID NO:43; or
the heavy chain variable region comprises: SEQ ID NO:34; and
the light chain variable region comprises: SEQ ID NO:44; or
the heavy chain variable region comprises: SEQ ID NO:34; and
the light chain variable region comprises: SEQ ID NO:45; or
the heavy chain variable region comprises: SEQ ID NO:37; and
the light chain variable region comprises: SEQ ID NO:44; or
the heavy chain variable region comprises: SEQ ID NO:37; and
the light chain variable region comprises: SEQ ID NO:45; or
the heavy chain variable region comprises: SEQ ID NO:34; and
the light chain variable region comprises: SEQ ID NO:46; or
the heavy chain variable region comprises: SEQ ID NO:34; and
the light chain variable region comprises: SEQ ID NO:47; or
the heavy chain variable region comprises: SEQ ID NO:37; and
the light chain variable region comprises: SEQ ID NO:46; or
the heavy chain variable region comprises: SEQ ID NO:37; and
the light chain variable region comprises: SEQ ID NO:47.
the heavy chain variable region comprises: SEQ ID NO:38; and
the light chain variable region comprises: SEQ ID NO:44; or
the heavy chain variable region comprises: SEQ ID NO:38; and
the light chain variable region comprises: SEQ ID NO:45; or
the heavy chain variable region comprises: SEQ ID NO:39; and
the light chain variable region comprises: SEQ ID NO:44; or
the heavy chain variable region comprises: SEQ ID NO:39; and
the light chain variable region comprises: SEQ ID NO:45; or
the heavy chain variable region comprises: SEQ ID NO:38; and
the light chain variable region comprises: SEQ ID NO:46; or
the heavy chain variable region comprises: SEQ ID NO:38; and
the light chain variable region comprises: SEQ ID NO:47; or
the heavy chain variable region comprises: SEQ ID NO:39; and
the light chain variable region comprises: SEQ ID NO:46; or
the heavy chain variable region comprises: SEQ ID NO:39; and
the light chain variable region comprises: SEQ ID NO:47.

10. The antibody of claim 8, wherein the antibody is humanized.

11. (canceled)

12. (canceled)

13. (canceled)

14. (canceled)

15. A method of reducing TGFβ activation in a human, the method comprising administering an antagonist of integrin αvβ8 to an individual in need thereof, wherein the antagonist is the antibody of claim 1, thereby reducing TGFβ activation in the individual.

16. (canceled)

17. (canceled)

18. (canceled)

19. (canceled)

20. A method of treating cancer in a human individual, the method comprising administering a sufficient amount of the antibody of claim 1 to the individual, thereby treating the cancer.

21. The method of claim 20, wherein the cancer is bladder cancer, colorectal cancer, glioblastoma, gynecologic cancer, liver cancer, head and neck cancer, kidney cancer, lung cancer, skin cancer, pancreas cancer, or sarcoma.

22. (canceled)

23. (canceled)

24. (canceled)

25. (canceled)

26. (canceled)

27. (canceled)

28. (canceled)

29. (canceled)

30. A method of enhancing an immune response to a viral infection in a human individual, the method comprising administering a sufficient amount of the antibody of claim 1 to the individual, thereby enhancing an immune response to the viral infection.

31. (canceled)

32. (canceled)

33. A pharmaceutical composition comprising the antibody of claim 1 in a pharmaceutically acceptable excipient.

34. A method of detecting the presence, absence, or quantity of human in a sample, the method comprising,
contacting the antibody of claim 1 to the sample, and
detecting or quantifying binding of the antibody to the sample.

35. A nucleic acid encoding a chimeric antigen receptor (CAR) polypeptide, wherein the CAR polypeptide comprises an extracellular domain comprising a single-chain antibody comprising:
(a) a heavy chain variable region comprising a heavy chain complementary determining region (HCDR) 1 comprising RQSMH (SEQ ID NO:1) or EQSMH (SEQ ID NO:7);
a HCDR 2 comprising RINTETGEPTYAQKFQG (SEQ ID NO:2);
a HCDR 3 comprising FYYGRD (S/T) (SEQ ID NO:3); and
(b) a light chain variable region comprising a light chain complementary determining region (LCDR) 1 comprising RSSQSLLHSRSRKNYLA (SEQ ID NO:4)
a LCDR2 comprising WASTRET (SEQ ID NO:5); and
a LCDR3 comprising KQSYNLLS (SEQ ID NO:6);
or
(c) a heavy chain variable region comprising a heavy chain complementary determining region (HCDR) 1 comprising SEQ ID NO:1 or SEQ ID NO:7;
a HCDR 2 comprising SEQ ID NO:2;
a HCDR 3 comprising SEQ ID NO:3; and
(d) a light chain variable region comprising a light chain complementary determining region (LCDR) 1 comprising KSSQSLLGRGDLGRLKKNALA (SEQ ID NO:8) or RSSQSLLRRGDLATIHGNALA (SEQ ID NO:9) or KSSQSLLRRGDLATIHGNALA (SEQ ID NO: 10) or RSSQSLLRRGDLATIHGNALA (SEQ ID NO:11);
a LCDR2 comprising SEQ ID NO:5; and
a LCDR3 comprising SEQ ID NO:6.

36. The nucleic acid of claim 35 wherein
the heavy chain variable region comprises: SEQ ID NO:34; and
the light chain variable region comprises: SEQ ID NO:35; or
the heavy chain variable region comprises: SEQ ID NO:34; and
the light chain variable region comprises: SEQ ID NO:36; or
the heavy chain variable region comprises: SEQ ID NO:37; and
the light chain variable region comprises: SEQ ID NO:35; or
the heavy chain variable region comprises: SEQ ID NO:37; and
the light chain variable region comprises: SEQ ID NO:36; or
the heavy chain variable region comprises: SEQ ID NO:38; and
the light chain variable region comprises: SEQ ID NO:35; or
the heavy chain variable region comprises: SEQ ID NO:38; and
the light chain variable region comprises: SEQ ID NO:36; or
the heavy chain variable region comprises: SEQ ID NO:39; and
the light chain variable region comprises: SEQ ID NO:35; or
the heavy chain variable region comprises: SEQ ID NO:39; and
the light chain variable region comprises: SEQ ID NO:36.

37. The nucleic acid of claim 35 wherein
the heavy chain variable region comprises: SEQ ID NO:34; and
the light chain variable region comprises: SEQ ID NO:40; or
the heavy chain variable region comprises: SEQ ID NO:34; and
the light chain variable region comprises: SEQ ID NO:41; or
the heavy chain variable region comprises: SEQ ID NO:37; and
the light chain variable region comprises: SEQ ID NO:40; or
the heavy chain variable region comprises: SEQ ID NO:37; and
the light chain variable region comprises: SEQ ID NO:41; or
the heavy chain variable region comprises: SEQ ID NO:34; and
the light chain variable region comprises: SEQ ID NO:42; or
the heavy chain variable region comprises: SEQ ID NO:34; and
the light chain variable region comprises: SEQ ID NO:43; or
the heavy chain variable region comprises: SEQ ID NO:37; and
the light chain variable region comprises: SEQ ID NO:42; or
the heavy chain variable region comprises: SEQ ID NO:37; and
the light chain variable region comprises: SEQ ID NO:43; or
the heavy chain variable region comprises: SEQ ID NO:38; and
the light chain variable region comprises: SEQ ID NO:40; or
the heavy chain variable region comprises: SEQ ID NO:38; and
the light chain variable region comprises: SEQ ID NO:41; or
the heavy chain variable region comprises: SEQ ID NO:39; and
the light chain variable region comprises: SEQ ID NO:40; or
the heavy chain variable region comprises: SEQ ID NO:39; and
the light chain variable region comprises: SEQ ID NO:41; or
the heavy chain variable region comprises: SEQ ID NO:38; and
the light chain variable region comprises: SEQ ID NO:42; or
the heavy chain variable region comprises: SEQ ID NO:38; and
the light chain variable region comprises: SEQ ID NO:43; or
the heavy chain variable region comprises: SEQ ID NO:39; and
the light chain variable region comprises: SEQ ID NO:42; or
the heavy chain variable region comprises: SEQ ID NO:39; and
the light chain variable region comprises: SEQ ID NO:43; or
the heavy chain variable region comprises: SEQ ID NO:34; and
the light chain variable region comprises: SEQ ID NO:44; or
the heavy chain variable region comprises: SEQ ID NO:34; and
the light chain variable region comprises: SEQ ID NO:45; or
the heavy chain variable region comprises: SEQ ID NO:37; and
the light chain variable region comprises: SEQ ID NO:44; or
the heavy chain variable region comprises: SEQ ID NO:37; and
the light chain variable region comprises: SEQ ID NO:45; or
the heavy chain variable region comprises: SEQ ID NO:34; and
the light chain variable region comprises: SEQ ID NO:46; or
the heavy chain variable region comprises: SEQ ID NO:34; and
the light chain variable region comprises: SEQ ID NO:47; or
the heavy chain variable region comprises: SEQ ID NO:37; and
the light chain variable region comprises: SEQ ID NO:46; or
the heavy chain variable region comprises: SEQ ID NO:37; and
the light chain variable region comprises: SEQ ID NO:47.
the heavy chain variable region comprises: SEQ ID NO:38; and
the light chain variable region comprises: SEQ ID NO:44; or
the heavy chain variable region comprises: SEQ ID NO:38; and
the light chain variable region comprises: SEQ ID NO:45; or
the heavy chain variable region comprises: SEQ ID NO:39; and
the light chain variable region comprises: SEQ ID NO:44; or
the heavy chain variable region comprises: SEQ ID NO:39; and
the light chain variable region comprises: SEQ ID NO:45; or
the heavy chain variable region comprises: SEQ ID NO:38; and
the light chain variable region comprises: SEQ ID NO:46; or
the heavy chain variable region comprises: SEQ ID NO:38; and
the light chain variable region comprises: SEQ ID NO:47; or
the heavy chain variable region comprises: SEQ ID NO:39; and
the light chain variable region comprises: SEQ ID NO:46; or
the heavy chain variable region comprises: SEQ ID NO:39; and
the light chain variable region comprises: SEQ ID NO:47.

38. (canceled)

39. An immune cell comprising the nucleic acid and expressing the CAR polypeptide of claim 35.

40. (canceled)


SEQUENCE LISTING (91 sequences)

SEQ ID NO: 1 - protein, 5 aa, synthetic construct
  REGION 1..5: note = Description of sequence: Description of sequence: Antibody component
RQSMH

SEQ ID NO: 2 - protein, 17 aa, synthetic construct
  REGION 1..17: note = Description of sequence: Description of sequence: Antibody component
RINTETGEPTYAQKFQG

SEQ ID NO: 3 - protein, 7 aa, synthetic construct
  REGION 1..7: note = Description of sequence: Description of sequence: Antibody component
  VARIANT 7: note = S may be replaced with T
FYYGRDS

SEQ ID NO: 4 - protein, 17 aa, synthetic construct
  REGION 1..17: note = Description of sequence: Description of sequence: Antibody component
RSSQSLLHSRSRKNYLA

SEQ ID NO: 5 - protein, 7 aa, synthetic construct
  REGION 1..7: note = Description of sequence: Description of sequence: Antibody component
WASTRET

SEQ ID NO: 6 - protein, 8 aa, synthetic construct
  REGION 1..8: note = Description of sequence: Description of sequence: Antibody component
KQSYNLLS

SEQ ID NO: 7 - protein, 5 aa, synthetic construct
  REGION 1..5: note = Description of sequence: Description of sequence: Antibody component
EQSMH

SEQ ID NO: 8 - protein, 21 aa, synthetic construct
  REGION 1..21: note = Description of sequence: Description of sequence: Antibody component
KSSQSLLGRGDLGRLKKNALA

SEQ ID NO: 9 - protein, 21 aa, synthetic construct
  REGION 1..21: note = Description of sequence: Description of sequence: Antibody component
RSSQSLLGRGDLGRLKKNALA

SEQ ID NO: 10 - protein, 21 aa, synthetic construct
  REGION 1..21: note = Description of sequence: Description of sequence: Antibody component
KSSQSLLRRGDLATIHGNALA

SEQ ID NO: 11 - protein, 21 aa, synthetic construct
  REGION 1..21: note = Description of sequence: Description of sequence: Antibody component
RSSQSLLRRGDLATIHGNALA

SEQ ID NO: 12 - protein, 30 aa, synthetic construct
  REGION 1..30: note = Description of sequence: Description of sequence: Antibody component
QVQLVQSGAEVKKPGASVKVSCKVSGYTLT

SEQ ID NO: 13 - protein, 14 aa, synthetic construct
  REGION 1..14: note = Description of sequence: Description of sequence: Antibody component
WVRQAPGKGLEWMG

SEQ ID NO: 14 - protein, 32 aa, synthetic construct
  REGION 1..32: note = Description of sequence: Description of sequence: Antibody component
RVTMTQDTSTDTAYMELSSLRSEDTAVYFCAI

SEQ ID NO: 15 - protein, 11 aa, synthetic construct
  REGION 1..11: note = Description of sequence: Description of sequence: Antibody component
WGQGTTVTVSS

SEQ ID NO: 16 - protein, 23 aa, synthetic construct
  REGION 1..23: note = Description of sequence: Description of sequence: Antibody component
EIVMTQSPATLSVSPGERVTLSC

SEQ ID NO: 17 - protein, 15 aa, synthetic construct
  REGION 1..15: note = Description of sequence: Description of sequence: Antibody component
WYQQKPGQAPRLLIY

SEQ ID NO: 18 - protein, 32 aa, synthetic construct
  REGION 1..32: note = Description of sequence: Description of sequence: Antibody component
GVPARFSGSGSGTEFTLTISSLQSEDFAVYYC

SEQ ID NO: 19 - protein, 10 aa, synthetic construct
  REGION 1..10: note = Description of sequence: Description of sequence: Antibody component
FGQGTKVEIK

SEQ ID NO: 20 - protein, 10 aa, synthetic construct
  REGION 1..10: note = Description of sequence: Description of sequence: Antibody component
FGQGTVLEIK

SEQ ID NO: 21 - protein, 32 aa, synthetic construct
  REGION 1..32: note = Description of sequence: Description of sequence: Antibody component
RVTMTEDTSTDTAYMELSSLRSEDTAVYFCAI

SEQ ID NO: 22 - protein, 116 aa, Homo sapiens
  REGION 1..116: note = Description of sequence: Description of sequence: Antibody component: huC6D4 VH
QIQLVQSGAEVKKPGASVKISCKASGYTFTDYSMHWVRQAPGQGLEWVARINTETGEPTFADDFRGRFTVTLDTSTSTAYLEIRSLRSDDTAVYFCAIFYYGRDTWGQGTTLTVSS

SEQ ID NO: 23 - protein, 113 aa, Homo sapiens
  REGION 1..113: note = Description of sequence: Description of sequence: Antibody component: huC6D4 VL
EIVMTQSPATLSVSPGERVTMSCKSSQSLLNSRSRKNYLAWYQQKPGQAPRLLIYWASTRESGVPARFSGSGSGTEFTLTISSVQSEDFAVYYCKQSYNLLSFGQGTVLEIKR

SEQ ID NO: 24 - protein, 116 aa, synthetic construct
  REGION 1..116: note = Description of sequence: Description of sequence: Antibody component: F12 VH
QIQLVQSGAEVKKPGASVKISCKASGYTFTKYSMHWVRQAPGQGLEWVARINTETGEPTFADDFRGRFTVTLDTSTSTAYLEIRSLRSDDTAVYFCAIFYYGRDTWGQGTTLTVSS

SEQ ID NO: 25 - protein, 113 aa, synthetic construct
  REGION 1..113: note = Description of sequence: Description of sequence: Antibody component: F12 VL
EIVMTQSPATLSVSPGERVTMSCKSSQSLLHSRSRKNYLAWYQQKPGQAPRLLIYWASTRESGVPARFSGSGSGTEFTLTISSVQSEDFAVYYCKQSYNLLSFGQGTVLEIKR

SEQ ID NO: 26 - protein, 117 aa, synthetic construct
  REGION 1..117: note = Description of sequence: Description of sequence: Antibody component: TGFb1RGD VL
DIVMTQSPSSLAVSAGEKVTMSCKSSQSLLRRGDLATIHGNALAWYQQKPGQSPRLLIYWASTRESGVPDRFTGSGSGTDFTLTISSVQAEDLAVYYCKQSYNLLSFGAGTKLELKA

SEQ ID NO: 27 - protein, 117 aa, synthetic construct
  REGION 1..117: note = Description of sequence: Description of sequence: Antibody component: C6D4-RGD3 VL
DIVMTQSPSSLAVSAGEKVTMSCKSSQSLLGRGDLGRLKKNALAWYQQKPGQSPRLLIYWASTRESGVPDRFTGSGSGTDFTLTISSVQAEDLAVYYCKQSYNLLSFGAGTKLELKA

SEQ ID NO: 28 - protein, 117 aa, Homo sapiens
  REGION 1..117: note = Description of sequence: Description of sequence: Antibody component: HuC6D4-RGD3 VL
EIVMTQSPATLSVSPGERVTMSCKSSQSLLGRGDLGRLKKNALAWYQQKPGQAPRLLIYWASTRESGVPARFSGSGSGTEFTLTISSVQSEDFAVYYCKQSYNLLSFGQGTVLEIKR

SEQ ID NO: 29 - protein, 121 aa, synthetic construct
  REGION 1..121: note = Description of sequence: Description of sequence: Antibody component: heavy_IGHV1-24_IGHD5-18_IGHJ6
QVQLVQSGAEVKKPGASVKVSCKVSGYTLTELSMHWVRQAPGKGLEWMGRFDPEDGETIYAQKFQGRVTMTEDISTDTAYMELSTLRSEDTAVYYCATGVWIREDYGMDVWGQGTTVTVSS

SEQ ID NO: 30 - protein, 120 aa, synthetic construct
  REGION 1..120: note = Description of sequence: Description of sequence: Antibody component: QEP17497.1 IGH c3222_heavy_IGHV1-24_IGHD3-9_IGHJ5
QVQLVQSGAEVKKPGASVKVSCKVSGYTLTELSMHWVRQAPGKGLEWMGGFDPEDGETIYAQKFQGRVTMTEDTSTDTAYMELSSLRSEDTAVYYCATVVRYFDWSIGGWGQGTLVTVSS

SEQ ID NO: 31 - protein, 98 aa, synthetic construct
  REGION 1..98: note = Description of sequence: Description of sequence: Antibody component: IGHV1-24*01
QVQLVQSGAEVKKPGASVKVSCKVSGYTLTELSMHWVRQAPGKGLEWMGGFDPEDGETIYAQKFQGRVTMTEDTSTDTAYMELSSLRSEDTAVYYCAT

SEQ ID NO: 32 - protein, 103 aa, synthetic construct
  REGION 1..103: note = Description of sequence: Description of sequence: Antibody component: QEP13372.1 IGL c3311_light_IGKV3-15_IGKJ1
EIVMTQSPATLSVSPGERATLSCRASQSVSSNLAWYQQKPGQAPRLLIYGASTRATGIPARFSGSGSGTEFTLTISSLQSEDFAVYYCQQSETFGQGTKVEIK

SEQ ID NO: 33 - protein, 88 aa, synthetic construct
  REGION 1..88: note = Description of sequence: Description of sequence: Antibody component: IGKV3-15*01
EIVMTQSPATLSVSPGERATLSCRASQSVSSNLAWYQQKPGQAPRLLIYGASTRATGIPARFSGSGSGTEFTLTISSLQSEDFAVYYC

SEQ ID NO: 34 - protein, 116 aa, synthetic construct
  REGION 1..116: note = Description of sequence: Description of sequence: Antibody component: H4.C8
QVQLVQSGAEVKKPGASVKVSCKVSGYTLTRQSMHWVRQAPGKGLEWMGRINTETGEPTYAQKFQGRVTMTQDTSTDTAYMELSSLRSEDTAVYFCAIFYYGRDTWGQGTTVTVSS

SEQ ID NO: 35 - protein, 113 aa, synthetic construct
  REGION 1..113: note = Description of sequence: Description of sequence: Antibody component: H3.C8
EIVMTQSPATLSVSPGERVTLSCRSSQSLLHSRSRKNYLAWYQQKPGQAPRLLIYWASTRETGVPARFSGSGSGTEFTLTISSLQSEDFAVYYCKQSYNLLSFGQGTKVEIKR

SEQ ID NO: 36 - protein, 113 aa, synthetic construct
  REGION 1..113: note = Description of sequence: Description of sequence: Antibody component: H3.C8
EIVMTQSPATLSVSPGERVTLSCRSSQSLLHSRSRKNYLAWYQQKPGQAPRLLIYWASTRETGVPARFSGSGSGTEFTLTISSLQSEDFAVYYCKQSYNLLSFGQGTVLEIKR

SEQ ID NO: 37 - protein, 116 aa, synthetic construct
  REGION 1..116: note = Description of sequence: Description of sequence: Antibody component: H4.C8
QVQLVQSGAEVKKPGASVKVSCKVSGYTLTRQSMHWVRQAPGKGLEWMGRINTETGEPTYAQKFQGRVTMTEDTSTDTAYMELSSLRSEDTAVYFCAIFYYGRDTWGQGTTVTVSS

SEQ ID NO: 38 - protein, 116 aa, synthetic construct
  REGION 1..116: note = Description of sequence: Description of sequence: Antibody component: H3.C8
QVQLVQSGAEVKKPGASVKVSCKVSGYTLTEQSMHWVRQAPGKGLEWMGRINTETGEPTYAQKFQGRVTMTQDTSTDTAYMELSSLRSEDTAVYFCAIFYYGRDTWGQGTTVTVSS

SEQ ID NO: 39 - protein, 116 aa, synthetic construct
  REGION 1..116: note = Description of sequence: Description of sequence: Antibody component: H3.C8
QVQLVQSGAEVKKPGASVKVSCKVSGYTLTEQSMHWVRQAPGKGLEWMGRINTETGEPTYAQKFQGRVTMTEDTSTDTAYMELSSLRSEDTAVYFCAIFYYGRDTWGQGTTVTVSS

SEQ ID NO: 40 - protein, 117 aa, synthetic construct
  REGION 1..117: note = Description of sequence: Description of sequence: Antibody component: H4.C8 RGD3 K
EIVMTQSPATLSVSPGERVTLSCKSSQSLLGRGDLGRLKKNALAWYQQKPGQAPRLLIYWASTRETGVPARFSGSGSGTEFTLTISSLQSEDFAVYYCKQSYNLLSFGQGTKVEIKR

SEQ ID NO: 41 - protein, 116 aa, synthetic construct
  REGION 1..116: note = Description of sequence: Description of sequence: Antibody component: H4.C8 RGD3 K
EIVMTQSPATLSVSPGERVTLSCKSQSLLGRGDLGRLKKNALAWYQQKPGQAPRLLIYWASTRETGVPARFSGSGSGTEFTLTISSLQSEDFAVYYCKQSYNLLSFGQGTVLEIKR

SEQ ID NO: 42 - protein, 117 aa, synthetic construct
  REGION 1..117: note = Description of sequence: Description of sequence: Antibody component: H4.C8 RGD3 R
EIVMTQSPATLSVSPGERVTLSCRSSQSLLGRGDLGRLKKNALAWYQQKPGQAPRLLIYWASTRETGVPARFSGSGSGTEFTLTISSLQSEDFAVYYCKQSYNLLSFGQGTKVEIKR

SEQ ID NO: 43 - protein, 117 aa, synthetic construct
  REGION 1..117: note = Description of sequence: Description of sequence: Antibody component: H4.C8 RGD3 R
EIVMTQSPATLSVSPGERVTLSCRSSQSLLGRGDLGRLKKNALAWYQQKPGQAPRLLIYWASTRETGVPARFSGSGSGTEFTLTISSLQSEDFAVYYCKQSYNLLSFGQGTVLEIKR

SEQ ID NO: 44 - protein, 117 aa, synthetic construct
  REGION 1..117: note = Description of sequence: Description of sequence: Antibody component: H4.C8 RGD1 K
EIVMTQSPATLSVSPGERVTLSCKSSQSLLRRGDLATIHGNALAWYQQKPGQAPRLLIYWASTRETGVPARFSGSGSGTEFTLTISSLQSEDFAVYYCKQSYNLLSFGQGTKVEIKR

SEQ ID NO: 45 - protein, 117 aa, synthetic construct
  REGION 1..117: note = Description of sequence: Description of sequence: Antibody component: H4.C8 RGD1 K
EIVMTQSPATLSVSPGERVTLSCKSSQSLLRRGDLATIHGNALAWYQQKPGQAPRLLIYWASTRETGVPARFSGSGSGTEFTLTISSLQSEDFAVYYCKQSYNLLSFGQGTVLEIKR

SEQ ID NO: 46 - protein, 117 aa, synthetic construct
  REGION 1..117: note = Description of sequence: Description of sequence: Antibody component: H4.C8 RGD1 R
EIVMTQSPATLSVSPGERVTLSCRSSQSLLRRGDLATIHGNALAWYQQKPGQAPRLLIYWASTRETGVPARFSGSGSGTEFTLTISSLQSEDFAVYYCKQSYNLLSFGQGTKVEIKR

SEQ ID NO: 47 - protein, 117 aa, synthetic construct
  REGION 1..117: note = Description of sequence: Description of sequence: Antibody component: H4.C8 RGD1 R
EIVMTQSPATLSVSPGERVTLSCRSSQSLLRRGDLATIHGNALAWYQQKPGQAPRLLIYWASTRETGVPARFSGSGSGTEFTLTISSLQSEDFAVYYCKQSYNLLSFGQGTVLEIKR

SEQ ID NO: 48 - protein, 411 aa, Homo sapiens
  REGION 1..411: note = Description of sequence: Description of sequence: Antibody component: Human TGFB3
MKMHLQRALVVLALLNFATVSLSLSTCTTLDFGHIKKKRVEAIRGQILSKLRLTSPPEPTVMTHVPYQVLALYNSTRELLEEMHGEREEGCTQENTESEYYAKEIHKFDMIQGLAEHNELAVCPKGITSKVFRFNVSSVEKNRTNLFRAEFRVLRVPNPSSKRNERIELFQILRPDEHIAKQRYIGGKNLPTRGTAEWLSFDVTDTVREWLLRRESNLGLEISIHCPCHTFQPNGDILENIHEVMEIKFKGVDNEDDHGRGDLGRLKKQKDHHNPHLILMMIPPHRLDNPGQGGQRKKRALDTNYCFRNLEENCCVRPLYIDFRQDLGWKWVHEPKGYYANFCSGPCPYLRSADTTHSTVLGLYNTLNPEASASPCCVPQDLEPLTILYYVGRTPKVEQLSNMVVKSCKCS

SEQ ID NO: 49 - protein, 10 aa, Homo sapiens
  REGION 1..10: note = Description of sequence: Description of sequence: Antibody component: Human TGFB3 RGD loop
GRGDLGRLKK

SEQ ID NO: 50 - protein, 389 aa, Homo sapiens
  REGION 1..389: note = Description of sequence: Description of sequence: Antibody component: Human TGFB1
MPPSGLRLLLLLLPLLWLLVLTPGRPAAGLSTCKTIDMELVKRKRIEAIRGQILSKLRLASPPSQGEVPPGPLPEAVLALYNSTRDRVAGESAEPEPEPEADYYAKEVTRVLMVETHNEIYDKFKQSTHSIYMFFNTSELREAVPEPVLLSRAELRLLRLKLKVEQHVELYQKYSNNSWRYLSNRLLAPSDSPEWLSFDVTGVVRQWLSRGGEIEGFRLSAHCSCDSRDNTLQVDINGFTTGRRGDLATIHGMNRPFLLLMATPLERAQHLQSSRHRRALDTNYCFSSTEKNCCVRQLYIDFRKDLGWKWIHEPKGYHANFCLGPCPYIWSDTQYSKVLALYNQHNPGASAAPCCVPQALEPLPIVYYVGRKPKVEQLSNMIVRSCKCS

SEQ ID NO: 51 - protein, 10 aa, Homo sapiens
  REGION 1..10: note = Description of sequence: Description of sequence: Antibody component: Human TGFB1 RGD loop
RRGDLATIHG

SEQ ID NO: 52 - protein, 680 aa, synthetic construct
  REGION 1..680: note = Description of sequence: Description of sequence: Antibody component: Integrin b8 subunit extracellular domain
MCGSALAFFTAAFVCLQNDRRGPASFLWAAWVFSLVLGLGQGEDNRCASSNAASCARCLALGPECGWCVQEDFISGGSRSERCDIVSNLISKGCSVDSIEYPSVHVIIPTENEINTQVTPGEVSIQLRPGAEANFMLKVHPLKKYPVDLYYLVDVSASMHNNIEKNSVGNDLSRKMAFFSRDFRLGFGSYVDKTVSPYISIHPERIHNQCSDYNLDCMPPHGYIHVLSLTENITEFEKAVHRQKISGNIDTPEGGFDAMLQAAVCESHIGWRKEAKRLLLVMTDQTSHLALDSKLAGIVVPNDGNCHLKNNVYVKSTTMEHPSLGQLSEKLIDNNINVIFAVQGKQFHWYKDLLPLLPGTIAGEIESKAANLNNLVVEAYQKLISEVKVQVENQVQGIYFNITAICPDGSRKPGMEGCRNVTSNDEVLFNVTVTMKKCDVTGGKNYAIIKPIGFNETAKIHIHRNCSCQCEDNRGPKGKCVDETFLDSKCFQCDENKCHFDEDQFSSESCKSHKDQPVCSGRGVCVCGKCSCHKIKLGKVYGKYCEKDDFSCPYHHGNLCAGHGECEAGRCQCFSGWEGDRCQCPSAAAQHCVNSKGQVCSGRGTCVCGRCECTDPRSIGRFCEHCPTCYTACKENWNCMQCLHPHNLSQAILDQCKTSCALMEQQHYVDQTSECFSSPS

SEQ ID NO: 53 - protein, 992 aa, synthetic construct
  REGION 1..992: note = Description of sequence: Description of sequence: Antibody component: Integrin av subunit extracellular domain
MAFPPRRRLRLGPRGLPLLLSGLLLPLCRAFNLDVDSPAEYSGPEGSYFGFAVDFFVPSASSRMFLLVGAPKANTTQPGIVEGGQVLKCDWSSTRRCQPIEFDATGNRDYAKDDPLEFKSHQWFGASVRSKQDKILACAPLYHWRTEMKQEREPVGTCFLQDGTKVEYAPCRSQDIDADGQGFCQGGFSIDFTKADRVLLGGPGSFYWQGQLISDQVAEIVSKYDPNVYSIKYNNQLATRTAQAIFDDSYLGYSVAVGDFNGDGIDDFVSGVPRAARTLGMVYIYDGKNMSSLYNFTGEQMAAYFGFSVAATDINGDDYADVFIGAPLFMDRGSDGKLQEVGQVSVSLQRASGDFQTTKLNGFEVFARFGSAIAPLGDLDQDGFNDIAIAAPYGGEDKKGIVYIFNGRSTGLNAVPSQILEGQWAARSMPPSFGYSMKGATDIDKNGYPDLIVGAFGVDRAILYRARPVITVNAGLEVYPSILNQDNKTCSLPGTALKVSCFNVRFCLKADGKGVLPRKLNFQVELLLDKLKQKGAIRRALFLYSRSPSHSKNMTISRGGLMQCEELIAYLRDESEFRDKLTPITIFMEYRLDYRTAADTTGLQPILNQFTPANISRQAHILLDCGEDNVCKPKLEVSVDSDQKKIYIGDDNPLTLIVKAQNQGEGAYEAELIVSIPLQADFIGVVRNNEALARLSCAFKTENQTRQVVCDLGNPMKAGTQLLAGLRFSVHQQSEMDTSVKFDLQIQSSNLFDKVSPVVSHKVDLAVLAAVEIRGVSSPDHVFLPIPNWEHKENPETEEDVGPVVQHIYELRNNGPSSFSKAMLHLQWPYKYNNNTLLYILHYDIDGPMNCTSDMEINPLRIKISSLQTTEKNDTVAGQGERDHLITKRDLALSEGDIHTLGCGVAQCLKIVCQVGRLDRGKSAILYVKSLLWTETFMNKENQNHSYSLKSSASFNVIEFPYKNLPIEDITNSTLVTTNVTWGIQPAPMPVP

SEQ ID NO: 54 - protein, 662 aa, synthetic construct
  REGION 1..662: note = Description of sequence: Description of sequence: Antibody component: LRRC32 (GARP)
MRPQILLLLALLTLGLAAQHQDKVPCKMVDKKVSCQVLGLLQVPSVLPPDTETLDLSGNQLRSILASPLGFYTALRHLDLSTNEISFLQPGAFQALTHLEHLSLAHNRLAMATALSAGGLGPLPRVTSLDLSGNSLYSGLLERLLGEAPSLHTLSLAENSLTRLTRHTFRDMPALEQLDLHSNVLMDIEDGAFEGLPRLTHLNLSRNSLTCISDFSLQQLRVLDLSCNSIEAFQTASQPQAEFQLTWLDLRENKLLHFPDLAALPRLIYLNLSNNLIRLPTGPPQDSKGIHAPSEGWSALPLSAPSGNASGRPLSQLLNLDLSYNEIELIPDSFLEHLTSLCFLNLSRNCLRTFEARRLGSLPCLMLLDLSHNALETLELGARALGSLRTLLLQGNALRDLPPYTFANLASLQRLNLQGNRVSPCGGPDEPGPSGCVAFSGITSLRSLSLVDNEIELLRAGAFLHTPLTELDLSSNPGLEVATGALGGLEASLEVLALQGNGLMVLQVDLPCFICLKRLNLAENRLSHLPAWTQAVSLEVLDLRNNSFSLLPGSAMGGLETSLRRLYLQGNPLSCCGNGWLAAQLHQGRVDVDATQDLICRFSSQEEVSLSHVRPEDCEKGGLKNINLIIILTFILVSAILLTTLAACCCVRRQKFNQQYKA

SEQ ID NO: 55 - protein, 5 aa, synthetic construct
  REGION 1..5: note = Description of sequence: Description of sequence: Antibody component: HC CDR1
  VARIANT 1: note = E may be replaced by R
EQSMH

SEQ ID NO: 56 - protein, 32 aa, synthetic construct
  REGION 1..32: note = Description of sequence: Description of sequence: Antibody component: F12 FR3
RFTVTLDTSTSTAYLEIRSLRSDDTAVYFCAI

SEQ ID NO: 57 - protein, 7 aa, synthetic construct
  REGION 1..7: note = Description of sequence: Description of sequence: Antibody component: HC CDR3
FYYGRDS

SEQ ID NO: 58 - protein, 7 aa, synthetic construct
  REGION 1..7: note = Description of sequence: Description of sequence: Antibody component: HC CDR3
FYYGRDT

SEQ ID NO: 59 - protein, 327 aa, Homo sapiens
  REGION 1..327: note = Description of sequence: Description of sequence: Antibody component: Human IgG4 (S228P R409K L445P) CH1-CH3
ASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSKLTVDKSRWQEGNVFSCSVMHEALHNHYTQKSLSLSPGK

SEQ ID NO: 60 - protein, 325 aa, Mus musculus
  REGION 1..325: note = Description of sequence: Description of sequence: Antibody component: Mouse IgG2a CH1-CH3
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKIVPRDCGCKPCICTVPEVSSVFIFPPKPKDVLTITLTPKVTCVVVDISKDDPEVQFSWFVDDVEVHTAQTQPREEQFNSTFRSVSELPIMHQDWLNGKEFKCRVNSAAFPAPIEKTISKTKGRPKAPQVYTIPPPKEQMAKDKVSLTCMITDFFPEDITVEWQWNGQPAENYKNTQPIMDTDGSYFVYSKLNVQKSNWEAGNTFTCSVLHEGLHNHHTEKSLSHSPGK

SEQ ID NO: 61 - protein, 325 aa, Mus musculus
  REGION 1..325: note = Description of sequence: Description of sequence: Antibody component: Mouse IgG1 (D265A) CH1-CH3
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKIVPRDCGCKPCICTVPEVSSVFIFPPKPKDVLTITLTPKVTCVVVAISKDDPEVQFSWFVDDVEVHTAQTQPREEQFNSTFRSVSELPIMHQDWLNGKEFKCRVNSAAFPAPIEKTISKTKGRPKAPQVYTIPPPKEQMAKDKVSLTCMITDFFPEDITVEWQWNGQPAENYKNTQPIMDTDGSYFVYSKLNVQKSNWEAGNTFTCSVLHEGLHNHHTEKSLSHSPGK

SEQ ID NO: 62 - protein, 115 aa, synthetic construct
  REGION 1..115: note = Description of sequence: Description of sequence: Antibody component: ADWA 11 2.4 Vh
EVQLVESGGGLVQPGGSLRLSCAASGFNIKDYYMNWVRQAPGKGLEWVGWIDPDQGNTIYEPKFQGRFTISADTSKNSAYLQMNSLRAEDTAVYYCARRLLMDYWGQGTLVTVSS

SEQ ID NO: 63 - protein, 113 aa, synthetic construct
  REGION 1..113: note = Description of sequence: Description of sequence: Antibody component: ADWA 11 2.4 light chain
DIQMTQSPSSLSASVGDRVTITCRSTKSLSHFNGNTYLFWYQQKPGKAPKRLIYYMSSLASGVPSRFSGSGSGTDFTLTISSLQPEDFATYYCQQSLEYPFTFGGGTKVEIKR

SEQ ID NO: 64 - protein, 120 aa, synthetic construct
  REGION 1..120: note = Description of sequence: Description of sequence: Antibody component: SRK-181 (Ab6) Vh
EVQLVESGGGLVQPGGSLRLSCTASGFTFSSFSMDWVRQAPGKGLEWVSYISPSADTIYYADSVKGRFTISRDNAKNTLYLQMNSLRAEDTAVYYCARGVLDYGDMLMPWGQGTLVTVSS

SEQ ID NO: 65 - protein, 109 aa, synthetic construct
  REGION 1..109: note = Description of sequence: Description of sequence: Antibody component: SRK-181 (Ab6) light chain
DIQMTQSPSSLSASVGDRVTITCQASQDITNYLNWYQQKPGKAPKLLIYDASNLETGVPSRFSGSGSGTDFTFTISSLQPEDIATYYCQQADNHPPWTFGGGTKVEIKR

SEQ ID NO: 66 - protein, 116 aa, synthetic construct; identical to SEQ ID NO: 38
  REGION 1..116: note = Description of sequence: Description of sequence: Antibody component: H3.12 VH

SEQ ID NO: 67 - protein, 113 aa, synthetic construct
  REGION 1..113: note = Description of sequence: Description of sequence: Antibody component: H3.12 VL
EIVMTQSPATLSVSPGERVTMSCRSSQSLLHSRTRKNYLAWYQQKPGQAPRLLIYWASTRETGVPARFSGSGSGTEFTLTISSLQSEDFAVYYCKQSYNLLSFGQGTVLEIKR

SEQ ID NO: 68 - protein, 116 aa, synthetic construct
  REGION 1..116: note = Description of sequence: Description of sequence: Antibody component: B4 VH
QVQLVQSGAEVKKPGASVKVSCKVSGYTLTKLSMHWVRQAPGKGLEWMGRINPEAGEPTYAQKFQGRVTMTVDTSTDTAYMELSSLRSEDTAVYYCAIFYYGRDSWGQGTTLTVSS

SEQ ID NO: 69 - protein, 113 aa, synthetic construct
  REGION 1..113: note = Description of sequence: Description of sequence: Antibody component: B4 VL
EIVMTQSPATLSVSPGERATLSCRASQSLLHSRTRKNYLAWYQQKPGQAPRLLIYWASTRATGIPARFSGSGSGTEFTLTISSLQSEDFAVYYCKQSYNLLSFGQGTVLEIKR

SEQ ID NO: 70 - protein, 116 aa, synthetic construct
  REGION 1..116: note = Description of sequence: Description of sequence: Antibody component: H1.1 VH
QVQLVQSGAEVKKPGASVKVSCKVSGYTLTKLSMHWVRQAPGKGLEWMGRINTEDGEPIYAQKFQGRVTMTEDTSTDTAYMELSSLRSEDTAVYYCAIFYYGRDSWGQGTTLTVSS

SEQ ID NO: 71 - protein, 113 aa, synthetic construct; identical to SEQ ID NO: 69
  REGION 1..113: note = Description of sequence: Description of sequence: Antibody component: H1.1 VK

SEQ ID NO: 72 - protein, 116 aa, synthetic construct
  REGION 1..116: note = Description of sequence: Description of sequence: Antibody component: A5 VH
QVQLVQSGAEVKKPGASVKVSCKVSGYTLTRHSMHWVRQAPGKGLEWMGRINPEAGEPTYAQKFQGRVTMTQDTSTDTAYLELSSLRSEDTAVYYCAIFYYGRDSWGQGTTLTVSS

SEQ ID NO: 73 - protein, 116 aa, synthetic construct
  REGION 1..116: note = Description of sequence: Description of sequence: Antibody component: A10 VH
QVQLVQSGAEVKKPGASVKVSCKVSGYTLTRQSMHWVRQAPGKGLEWMGRINTETGEPTYAQKFQGRVTMTEDTSTDTAYMELSSLRSEDTAVYYCAIFYYGRDSWGQGTTLTVSS

SEQ ID NO: 74 - protein, 113 aa, synthetic construct
  REGION 1..113: note = Description of sequence: Description of sequence: Antibody component: A10 VL
EIVMTQSPATLSVSPGERATLSCRSSQSLLHSRTRKNYLAWYQQKPGQAPRLLIYWASTRDTGIPARFSGSGSGTEFTLTISSLQSEDFAVYYCKQSYNLLSFGQGTVLEIKR

SEQ ID NO: 75 - protein, 116 aa, synthetic construct
  REGION 1..116: note = Description of sequence: Description of sequence: Antibody component: A12 VH
QVQLVQSGAEVKKPGASVKVSCKVSGYTLTEQSMHWVRQAPGKGLEWMGRINTETGEPTYAQKFQGRVTMTQDTSTDTAYMELSSLRSEDTAVYYCAIFYYGRDSWGQGTTLTVSS

SEQ ID NO: 76 - protein, 113 aa, synthetic construct
  REGION 1..113: note = Description of sequence: Description of sequence: Antibody component: A12 VL
EIVMTQSPATLSVSPGERATLSCRSSQSLLHSRTRKNYLAWYQQKPGQAPRLLIYWASTRATGIPARFSGSGSGTEFTLTISSLQSEDFAVYYCKQSYNLLSFGQGTVLEIKR

SEQ ID NO: 77 - protein, 116 aa, synthetic construct
  REGION 1..116: note = Description of sequence: Description of sequence: Antibody component: C12 VH
QVQLVQSGAEVKKPGASVKVSCKVSGYTLTEQSMHWVRQAPGKGLEWMGRINTENGEPTYAQKFQGRVTMTQDTSTDTAYLELSSLRSEDTAVYYCAIFYYGRDSWGQGTTLTVSS

SEQ ID NO: 78 - protein, 113 aa, synthetic construct; identical to SEQ ID NO: 76
  REGION 1..113: note = Description of sequence: Description of sequence: Antibody component: C12 VL

SEQ ID NO: 79 - protein, 116 aa, Homo sapiens
  REGION 1..116: note = Description of sequence: Description of sequence: Antibody component: HuF12 Fr1 human VH
QVQLVQSGAEVKKPGASVKVSCKVSGYTFTKYSMHWVRQAPGQGLEWVARINTETGEPTFADDFRGRFTVTLDTSTSTAYLEIRSLRSDDTAVYFCAIFYYGRDTWGQGTTLTVSS

SEQ ID NO: 80 - protein, 116 aa, synthetic construct
  REGION 1..116: note = Description of sequence: Description of sequence: Antibody component: B4 Fr1:CDR1:F12 VH
QVQLVQSGAEVKKPGASVKVSCKVSGYTFTKLSMHWVRQAPGQGLEWVARINTETGEPTFADDFRGRFTVTLDTSTSTAYLEIRSLRSDDTAVYFCAIFYYGRDTWGQGTTLTVSS

SEQ ID NO: 81 - protein, 116 aa, synthetic construct
  REGION 1..116: note = Description of sequence: Description of sequence: Antibody component: A5 Fr1:CDR1:F12 VH
QVQLVQSGAEVKKPGASVKVSCKVSGYTFTRHSMHWVRQAPGQGLEWVARINTETGEPTFADDFRGRFTVTLDTSTSTAYLEIRSLRSDDTAVYFCAIFYYGRDTWGQGTTLTVSS

SEQ ID NO: 82 - protein, 116 aa, synthetic construct; identical to SEQ ID NO: 80
  REGION 1..116: note = Description of sequence: Description of sequence: Antibody component: A10 Fr1:CDR1:F12 VH

SEQ ID NO: 83 - protein, 116 aa, synthetic construct
  REGION 1..116: note = Description of sequence: Description of sequence: Antibody component: A12 Fr1:CDR1:F12 VH
QVQLVQSGAEVKKPGASVKVSCKVSGYTFTEQSMHWVRQAPGQGLEWVARINTETGEPTFADDFRGRFTVTLDTSTSTAYLEIRSLRSDDTAVYFCAIFYYGRDTWGQGTTLTVSS

SEQ ID NO: 84 - protein, 121 aa, synthetic construct
  REGION 1..121: note = Description of sequence: Description of sequence: Antibody component: MHG-8 VH
QVQLKESGPGLVAPSQSLSITCTVSGFSLTGYGINWVRQPPGKGLEWLGMIWSDGSTDYNSVLTSRLRISKDNSNSQVFLKMNSLQVDDTARYYCARDRNYYDYDGAMDYWGQGTSVTVSS

SEQ ID NO: 85 - protein, 107 aa, synthetic construct
  REGION 1..107: note = Description of sequence: Description of sequence: Antibody component: MHG-8 VL
DIQVTQSSSYLSVSLGDRVTITCKASDHIKNWLAWYQQKPGIAPRLLVSGATSLEAGVPSRFSGSGSGKNFTLSITSLQTEDVATYYCQQYWSTPWTFGGGTTLEIR

SEQ ID NO: 86 - protein, 325 aa, Mus musculus; identical to SEQ ID NO: 60
  REGION 1..325: note = Description of sequence: Description of sequence: Antibody component: Mouse IgG1 CH1-CH3

SEQ ID NO: 87 - protein, 255 aa, synthetic construct
  REGION 1..255: note = Description of sequence: Description of sequence: Antibody component: F12 scFV
QIQLVQSGAEVKKPGASVKISCKASGYTFTKYSMHWVRQAPGQGLEWVARINTETGEPTFADDFRGRFTVTLDTSTSTAYLEIRSLRSDDTAVYFCAIFYYGRDTWGQGTTLTVSSVDSKSGGSTSGSGKPGSGEGSSGSAREIVMTQSPATLSVSPGERVTMSCKSSQSLLHSRSRKNYLAWYQQKPGQAPRLLIYWASTRESGVPARFSGSGSGTEFTLTISSVQSEDFAVYYCKQSYNLLSFGQGTVLEIKR

SEQ ID NO: 88 - protein, 255 aa, synthetic construct
  REGION 1..255: note = Description of sequence: Description of sequence: Antibody component: H4C8 scFV
QVQLVQSGAEVKKPGASVKVSCKVSGYTLTRQSMHWVRQAPGKGLEWMGRINTETGEPTYAQKFQGRVTMTQDTSTDTAYMELSSLRSEDTAVYFCAIFYYGRDTWGQGTTVTVSSVDSKSGGSTSGSGKPGSGEGSSGSAREIVMTQSPATLSVSPGERVTLSCRSSQSLLHSRSRKNYLAWYQQKPGQAPRLLIYWASTRETGVPARFSGSGSGTEFTLTISSLQSEDFAVYYCKQSYNLLSFGQGTVLEIKR

SEQ ID NO: 89 - protein, 217 aa, Mus musculus
  REGION 1..217: note = Description of sequence: Description of sequence: Antibody component: Murine CD28:CD3epsilon
IEFMYPPPYLDNERSNGTIIHIKEKHLCHTQSSPKLFWALVVVAGVLFCYGLLVTVALCVIWTNSRRNRGGQSDYMNMTPRRPGLTRKPYQPYAPARDFAAYRPRAKFSRSAETAANLQDPNQLFNELNLGRREEFDVLEKKRARDPEMGGKQQRRRNPQEGVYNALQKDKMAEAYSEIGTKGERRRGKGHDGLFQGLSTATKDTFDALHMQTLAPR

SEQ ID NO: 90 - protein, 472 aa, synthetic construct
  REGION 1..472: note = Description of sequence: Description of sequence: Antibody component: F12:CD28:CD3epsilon
QIQLVQSGAEVKKPGASVKISCKASGYTFTKYSMHWVRQAPGQGLEWVARINTETGEPTFADDFRGRFTVTLDTSTSTAYLEIRSLRSDDTAVYFCAIFYYGRDTWGQGTTLTVSSVDSKSGGSTSGSGKPGSGEGSSGSAREIVMTQSPATLSVSPGERVTMSCKSSQSLLHSRSRKNYLAWYQQKPGQAPRLLIYWASTRESGVPARFSGSGSGTEFTLTISSVQSEDFAVYYCKQSYNLLSFGQGTVLEIKRIEFMYPPPYLDNERSNGTIIHIKEKHLCHTQSSPKLFWALVVVAGVLFCYGLLVTVALCVIWTNSRRNRGGQSDYMNMTPRRPGLTRKPYQPYAPARDFAAYRPRAKFSRSAETAANLQDPNQLFNELNLGRREEFDVLEKKRARDPEMGGKQQRRRNPQEGVYNALQKDKMAEAYSEIGTKGERRRGKGHDGLFQGLSTATKDTFDALHMQTLAPR

SEQ ID NO: 91 - protein, 472 aa, synthetic construct
  REGION 1..472: note = Description of sequence: Description of sequence: Antibody component: H4C8:CD28:CD3epsilon
QVQLVQSGAEVKKPGASVKVSCKVSGYTLTRQSMHWVRQAPGKGLEWMGRINTETGEPTYAQKFQGRVTMTQDTSTDTAYMELSSLRSEDTAVYFCAIFYYGRDTWGQGTTVTVSSVDSKSGGSTSGSGKPGSGEGSSGSAREIVMTQSPATLSVSPGERVTLSCRSSQSLLHSRSRKNYLAWYQQKPGQAPRLLIYWASTRETGVPARFSGSGSGTEFTLTISSLQSEDFAVYYCKQSYNLLSFGQGTVLEIKRIEFMYPPPYLDNERSNGTIIHIKEKHLCHTQSSPKLFWALVVVAGVLFCYGLLVTVALCVIWTNSRRNRGGQSDYMNMTPRRPGLTRKPYQPYAPARDFAAYRPRAKFSRSAETAANLQDPNQLFNELNLGRREEFDVLEKKRARDPEMGGKQQRRRNPQEGVYNALQKDKMAEAYSEIGTKGERRRGKGHDGLFQGLSTATKDTFDALHMQTLAPR